(19) United States
(12) Patent Application Publication
(10) Pub. No.: US 2026/0256485 A1
(43) Pub. Date: Sep. 3, 2026

(54) Apparatuses, Methods and Systems for the Percutaneous Treatment of Cholelithiasis

(71) Applicant: Inari Medical, Inc., Irvine, CA (US)

(72) Inventors: Benjamin E. Merritt, San Clemente, CA (US); Mikayla Ann Barkley, Irvine, CA (US); John Coleman Thress, Oceanside, CA (US); Jared Shimizu, Tustin, CA (US); Rachel Wong, Santa Ana, CA (US); Thomas Brodt, Long Beach, CA (US); Brian Merritt, San Clemente, CA (US); Philippe Marchand, Pointe-Claire (CA); Heidi Lynne Silk, Costa Mesa, CA (US); Venkat P. Tummala, South Lake, TX (US); Behraam Hussain Baqai, Irvine, CA (US); Evan Hunter, Mission Viejo, CA (US); Catherine S. Lee, Costa Mesa, CA (US); Brandon James Conrad, Laguna Niguel, CA (US)

(21) Appl. No.: 18/879,759

(22) PCT Filed: Jun. 29, 2023
(86) PCT No.: PCT/US2023/026648

§ 371 Date: Dec. 28, 2024

Related U.S. Application Data
(60) Provisional application No. 63/357,406, filed on Jun. 30, 2022.

(51) Int. Cl. A61B 17/221; A61B 17/00; A61B 17/22; A61B 17/34

(52) U.S. Cl. CPC A61B 17/221; A61B 17/3415; A61B 2017/00398; A61B 2017/22069; A61B 2017/22077; A61B 2017/22079; A61B 2217/005

(57) ABSTRACT

Systems, apparatuses and methods for percutaneous removal of gallstones and gallstone fragments for the treatment of cholelithiasis are disclosed. A representative system includes an access sheath; an aspiration device removably coupleable to the access sheath; and a catheter removably insertable into the access sheath. A representative system may also include a crushing or fracturing control apparatus and/or a crushing or fracturing instrument. A representative access sheath includes a tubular shaft and a proximal hub having one or more valves. A representative aspiration device includes a syringe tube body; an extended distal tip, which may also have a valve; and a plunger moveable within an interior lumen of the syringe tube body. A representative catheter includes an outer catheter tube; an inner catheter manipulation shaft moveable within a lumen of the outer catheter tube; and a basket or cage having a plurality of longitudinal struts.

### CROSS-REFERENCE TO RELATED APPLICATIONS

This application is a U.S. national phase under 35 U.S.C. Section 371 and claims the benefit of and priority to International Application No. PCT/US2023/026648, filed Jun. 29, 2023, inventors Benjamin E. Merritt et al., titled “Apparatuses, Methods and Systems for the Percutaneous Treatment of Cholelithiasis”, which is a nonprovisional of and claims priority to and the benefit of United States Provisional Patent Application No. 63/357,406, filed Jun. 30, 2022, inventors Ben Merritt et al., titled “Apparatus, Method and System for Percutaneous Treatment of Cholelithiasis”, which are commonly assigned herewith, incorporated herein by reference with the same full force and effect as if set forth in their entireties herein, and with priority claimed for all commonly disclosed subject matter.

### FIELD OF THE INVENTION

The present invention, in general, relates to the medical and surgical treatment of cholelithiasis, and more particularly, relates to apparatuses, methods and systems for the percutaneous removal of gallstones for the treatment of cholelithiasis.

### BACKGROUND OF THE INVENTION

The presence of gallstones in the biliary tract, referred to as cholelithiasis, including the presence of one or more gallstones in the gallbladder or the common bile duct, for example, impacts approximately two to four million patients annually. The standard treatment method is surgical intervention and removal of the gallbladder, referred to as a cholecystectomy, which is generally successful and typically proceeds without complications.

Approximately 50,000 patients are non-surgical candidates, however, and need an alternative treatment. These patients typically receive transhepatic drains to help with inflammation and other symptoms of cholelithiasis. A non-surgical patient may need to live the rest of their life with the transhepatic drain in place. This effectively permanent drain negatively impacts the patient's quality of life and can limit their daily functions.

Attempts have been made to treat cholelithiasis utilizing instruments which have been designed for other medical and surgical procedures and treatment methodologies. This includes, for example and without limitation, endoscopes having various grabbing and crushing tools, lithotripters, and lasers, and repurposing them for use in treating cholelithiasis. Such endoscopes and other instruments, however, are not configured, sized, or shaped appropriately for the removal of gallstones which may be as large as 30 mm and quite hard, for example. In addition, such endoscopes and other instruments are not designed for the specific environment of a gallbladder, which has flexible, compliant walls and undergoes dramatic changes in shape and size if drained or irrigated.

Accordingly, a need remains for apparatuses, methods and systems specifically designed for the percutaneous removal of gallstones for the treatment of cholelithiasis. Such apparatuses, methods and systems should be capable of percutaneous and transhepatic placement into an affected gallbladder, and capable of removing any gallstones in the gallbladder, the common bile duct, or other biliary tract locations. Such apparatuses, methods and systems should be comparatively low cost, particularly in comparison to costs associated with the surgical treatment of cholelithiasis and subsequent inpatient hospitalization. Such apparatuses, methods and systems should be capable of operation in a typical interventional radiology suite, rather than a surgical operating room. Such apparatuses, methods and systems should further provide a treatment option for patients who are not surgical candidates, and thereby improve the quality of life which would otherwise not be available to such patients.

### SUMMARY OF THE INVENTION

The exemplary or representative embodiments of the present invention provide numerous advantages. Various representative embodiments provide apparatuses, methods and systems specifically designed for the percutaneous removal of gallstones for the treatment of cholelithiasis, also referred to as a cholelithotomy. The representative embodiments are capable of percutaneous or percutaneous and transhepatic placement directly into an affected gallbladder, and capable of removing any gallstones in the gallbladder. The representative apparatuses, methods and systems are comparatively low cost, particularly in comparison to costs associated with the surgical treatment of cholelithiasis and subsequent inpatient hospitalization. The representative apparatus, method and system are capable of operation in a typical interventional radiology suite, rather than a surgical operating room. The representative embodiments further provide a treatment option of removing gallstones for patients who are not surgical candidates, and further avoiding the discomfort and other associated problems of a permanent drain, for example, thereby improving the quality of life which would otherwise not be available to such patients.

A representative system embodiment for percutaneous removal of gallstones for the treatment of cholelithiasis is disclosed, with the representative system comprising: an access sheath; an aspiration device removably coupleable to the access sheath; and a catheter removably insertable into the access sheath.

In a representative embodiment, the access sheath comprises: a tubular shaft having a first, proximal end and a second, distal end, the tubular shaft having a longitudinal axis and a central lumen arranged longitudinally; and a proximal hub coupled to the first end of the tubular shaft. The second end of the tubular shaft may be angled or beveled.

In a representative embodiment, the tubular shaft comprises: an inner shaft wall forming the central lumen; an outer shaft wall arranged coaxially with and spaced apart radially from the inner shaft wall to form, in between the inner and outer shaft walls, one or more peripheral lumens; and an inflation lumen arranged between the inner and outer shaft walls.

In a representative embodiment, the outer shaft wall further comprises: a plurality of infusion ports and at least one inflation port, the plurality of infusion ports arranged radially around a periphery of the outer shaft wall and spaced apart from the second, distal end, the inflation lumen arranged spaced apart from the second, distal end, and the plurality of infusion ports in fluid communication with the one or more peripheral lumens.

In another representative embodiment, the access sheath further comprises: an access sheath tip coupled to the second, distal end of the tubular shaft, the access sheath tip having a beveled tip and a plurality of infusion ports, the plurality of infusion ports arranged radially and spaced apart from the beveled tip and in fluid communication with the one or more peripheral lumens, the access sheath tip further comprising a plurality of tabs insertable between the inner shaft wall and the outer shaft wall.

In a representative embodiment, the access sheath may further comprise: an inflatable sealing and anchoring balloon arranged spaced apart from the second, distal end and proximally to the plurality of infusion ports, the inflatable sealing and anchoring balloon coupled radially around the outer wall, the inflatable sealing and anchoring balloon having a balloon lumen arranged in fluid communication with the inflation lumen. In a representative embodiment, the access sheath may further comprise: an adjustable stopper arranged spaced apart proximally from the inflatable sealing and anchoring balloon and coupled radially around the outer wall.

In a representative embodiment, the central lumen forms an aspiration channel for aspiration of fluid and one or more gallstones or gallstone fragments from a gallbladder of the human or veterinary subject and the one or more peripheral lumens and plurality of infusion ports collectively form an infusion channel for infusion of fluid into the gallbladder of the human or veterinary subject.

In a representative embodiment, the proximal hub comprises: a hub housing having a hub lumen, the hub lumen arranged in fluid communication with the central lumen; a filter assembly removably coupleable to the hub housing, the filter assembly arranged in fluid communication with the central lumen; a first port integrally formed with or coupled to the hub housing, the first port arranged in fluid communication with the one or more peripheral lumens; and a second port integrally formed with or coupled to the hub housing, the second port arranged in fluid communication with the inflation lumen.

In a representative embodiment, the filter assembly comprises: a filter canister removably coupleable to the housing, the filter canister having a filter canister lumen; and a filter arranged within the filter canister and in fluid communication with the hub lumen, the filter forming a filter chamber within the filter canister. In a representative embodiment, the filter assembly is linearly arranged with or along the longitudinal axis.

In a representative embodiment, the proximal hub may further comprise: an infusion path tubing integrally formed with or coupled to the first port, the infusion path tubing arranged in fluid communication with the one or more peripheral lumens; a first valve integrally formed with or coupled to the infusion path tubing; a second valve integrally formed with or coupled to the hub housing and in fluid communication with the filter canister lumen; a connector coupled to the first valve and to the second valve, the connector removably coupleable to an aspiration device. In a representative embodiment, the proximal hub may further comprise: a third valve integrally formed with or coupled between the infusion path tubing and the first port. In a representative embodiment, the first valve and the second valve each comprise at least one valve or adapter selected from the group consisting of: a pressure cracking valve; a duckbill valve; a cross-slit valve; a stopcock valve, a gate valve, a rotary valve; a ball valve; a luer valve; a Tuohy-Borst adapter; and combinations thereof. In another representative embodiment, the proximal hub may further comprise: a third port integrally formed with or coupled to the hub housing, the third port arranged in fluid communication with the central lumen for insertion of a catheter into the central lumen.

In another representative embodiment, the proximal hub may further comprise: an infusion path tubing integrally formed with or coupled to the first port, the infusion path tubing arranged in fluid communication with the one or more peripheral lumens; one or more first valves integrally formed with or coupled to the infusion path tubing; a second valve integrally formed with or coupled to the filter assembly or to the hub housing and in fluid communication with the filter canister lumen; a return path tubing integrally formed with or coupled to the second valve; a connecting tubing having a third valve coupled between and in fluid communication with the infusion path tubing and the return path tubing; a first connector coupled to the infusion path tubing; and a second connector coupled to the return path tubing.

In a representative embodiment, the aspiration device comprises: a first aspiration device removably coupleable to the first connector, the first aspiration device comprising a first syringe tube body and a first plunger moveable within the first syringe tube body; and a second aspiration device removably coupleable to the second connector, the second aspiration device comprising a second syringe tube body and a second plunger moveable within the second syringe tube body.

In a representative embodiment, the system may further comprise: a first rack gear coupled to the first plunger; a second rack gear coupled to the second plunger; a pinion gear abutting and moveably engaging the first and second rack gears, the pinion gear arranged to advance the first plunger within the first syringe tube body while concurrently retracting the second plunger from the second syringe tube body and to advance the second plunger within the second syringe tube body while concurrently retracting the first plunger from the first syringe tube body; and a clamping assembly coupled to the pinion gear and to the first and second syringe tube bodies.

In another representative embodiment, the system may further comprise: a pump coupled to the first and second plungers, the pump arranged to advance the first plunger within the first syringe tube body while concurrently retracting the second plunger from the second syringe tube body and to advance the second plunger within the second syringe tube body while concurrently retracting the first plunger from the first syringe tube body.

In another representative embodiment, the proximal hub may comprise: a hub housing having a hub lumen, the hub lumen arranged in fluid communication with the central lumen; a first port integrally formed with or coupled to the hub housing, the first port arranged in fluid communication with the one or more peripheral lumens; a second port integrally formed with or coupled to the hub housing, the second port arranged in fluid communication with the hub lumen; a filter assembly removably coupleable to the second port, the filter assembly arranged in fluid communication with the hub lumen; and a third port integrally formed with or coupled to the hub housing, the third port arranged in fluid communication with the inflation lumen. In a representative embodiment, the filter assembly is arranged spaced apart transversely from the hub lumen, the longitudinal axis and central lumen.

In a representative embodiment, the proximal hub may further comprise: a first valve integrally formed with or coupled to the first port; an infusion path tubing integrally formed with or coupled to the first valve, the infusion path tubing arranged in fluid communication with the one or more peripheral lumens; a second valve coupled to the second port and to the filter assembly, the second valve in fluid communication with the hub lumen; a return path tubing coupled to the filter assembly, the return path tubing arranged in fluid communication with the filter canister lumen; and a connector coupled to the infusion path tubing and to the return path tubing, the connector removably coupleable to an aspiration device.

In a representative embodiment, the first valve and the second valve each comprise at least one valve or adapter selected from the group consisting of: a pressure cracking valve; a duckbill valve; a cross-slit valve; a stopcock valve, a gate valve, a rotary valve; a ball valve; a luer valve; a Tuohy-Borst adapter; and combinations thereof.

In a representative embodiment, the hub lumen is linearly arranged with or along the longitudinal axis and the filter assembly, the return path tubing, and the infusion path tubing are arranged spaced apart transversely from the hub lumen, the longitudinal axis and central lumen.

In a representative embodiment, the proximal hub may further comprise: a third valve coupled to the hub housing and linearly arranged with or along the longitudinal axis. In a representative embodiment, third valve may be a hemostasis valve having a button actuator, and wherein a catheter is insertable in-line with or along the longitudinal axis through the hemostasis valve and into the hub lumen and the central lumen.

In another representative embodiment, the tubular shaft comprises: an outer shaft wall having a plurality of infusion ports arranged radially around a periphery of the outer shaft wall and spaced apart from the second, distal end; and an inner shaft wall forming the central lumen, the inner shaft wall arranged coaxially with and spaced apart radially from the outer shaft wall to form, in between the inner and outer shaft walls, one or more peripheral lumens, the inner shaft wall removably coupleable to the outer shaft wall, the inner shaft wall having a flared distal end.

In another representative embodiment, the access sheath may further comprise: an inflatable sealing and anchoring balloon arranged spaced apart from the second, distal end and proximally to the plurality of infusion ports, the inflatable sealing and anchoring balloon coupled radially around the outer wall, the inflatable sealing and anchoring balloon having a balloon lumen arranged in fluid communication with an inflation lumen, the inflation lumen arranged on an exterior of the outer shaft wall.

In another representative embodiment, the proximal hub comprises: a first proximal hub section coupled to the outer shaft wall; a first port integrally formed with or coupled to the first proximal hub section the first port arranged in fluid communication with the inflation lumen; a second proximal hub section coupled to the inner shaft wall and removably coupleable to the first proximal hub section; a second port integrally formed with or coupled to the second proximal hub section, the second port arranged in fluid communication with the one or more peripheral lumens; and an in-line or linearly arranged port or connector integrally formed with or coupled to the second proximal hub section.

In a representative embodiment, the central lumen has an inner diameter between 8-10 mm (24-30 Fr), with the access sheath having a length between 12 cm and 18 cm. In another representative embodiment, the central lumen has an inner diameter between 8 mm to 12 mm, with the access sheath having a length between 5 cm and 40 cm.

In another representative embodiment, the proximal hub comprises: a housing having a hub lumen, the hub lumen continuous with and in fluid communication with the shaft lumen; a first valve arranged in the hub lumen; and a second valve arranged in the hub lumen, the second valve spaced apart from the first valve to form a hub chamber between the first and second valves. In a representative embodiment, the first valve and the second valve are each a self-sealing valve.

In a representative embodiment, the proximal hub may further comprise: one or more access ports integrally formed with the housing and arranged distally to the first valve, the one or more access ports in fluid communication with the hub lumen; and a central, in-line access port in fluid communication with the hub lumen.

In a representative embodiment, the proximal hub may further comprise: one or more third valves arranged within or coupleable to the one or more access ports. In a representative embodiment, the proximal hub may further comprise: a first coupling comprising a first tab recess and a second tab recess. In a representative embodiment, the proximal hub may further comprise: a first coupling comprising a connector having a flexible ring or gasket.

In a representative embodiment, the proximal hub may comprise: a housing having a hub lumen, the hub lumen continuous with and in fluid communication with the shaft lumen; and at least one valve arranged in the hub lumen. In a representative embodiment, the proximal hub may further comprise: one or more access ports integrally formed with the housing and arranged distally to the first valve, the one or more access ports in fluid communication with the hub lumen; and a central, in-line access port in fluid communication with the hub lumen. In a representative embodiment, the proximal hub may further comprise: a first coupling comprising a first tab recess and a second tab recess. In a representative embodiment, the proximal hub may further comprise: a first coupling comprising a connector having a flexible ring or gasket.

In a representative embodiment, the access sheath may further comprise: a flexible, self-expanding member coupled to the proximal hub. In a representative embodiment, the tubular shaft may further comprise one or more interior, longitudinal partitions forming a plurality of separate shaft lumens. In a representative embodiment, the access sheath may be curved, tapered, or angled.

In a representative embodiment, the aspiration device comprises: a syringe tube body having an interior lumen; an extended distal tip coupled to or integrally formed with the syringe tube body; and a plunger moveable within the interior lumen of the syringe tube body.

In a representative embodiment, the aspiration device further comprises a first syringe valve (or adapter) coupled to the extended distal tip. In another representative embodiment, the aspiration device further comprises a second syringe valve (or adapter) coupled to the syringe tube body. In a representative embodiment, the first syringe valve and the second syringe valve each comprise at least one valve (or adapter) selected from the group consisting of: a duckbill valve; a cross-slit valve; a stopcock valve, a gate valve, a rotary valve; a ball valve; a luer valve; a Tuohy-Borst adapter; and combinations thereof. In another representative embodiment, the aspiration device does not include a valve.

In a representative embodiment, the plunger comprises a plunger body having a plunger lumen extending longitudinally within the plunger body; a plunger handle coupled to the plunger body. In a representative embodiment, the catheter is further removably insertable through the plunger lumen and into a shaft lumen of the access sheath. In such a representative embodiment, the plunger may comprise: a plunger body having a plunger lumen extending longitudinally within the plunger body; a plunger access port linearly arranged with and in fluid communication with the plunger lumen; and a plunger handle coupled to the plunger body.

In a representative embodiment, the catheter has a longitudinal dimension and a transverse dimension, wherein the catheter comprises: an outer catheter tube having an outer catheter lumen and an outer catheter tube distal end; an inner catheter manipulation shaft moveable longitudinally and rotatably within the outer catheter lumen, the inner catheter manipulation shaft having an inner catheter lumen and an inner catheter shaft distal end; and a basket or cage coupled to the inner catheter manipulation shaft, the basket or cage comprising a plurality of struts arranged longitudinally. In a representative embodiment, one or more struts of the plurality of struts are coupled to each other at a first end of the basket or cage and one or more struts of the plurality of struts are coupled to the distal end of the inner catheter manipulation shaft at a second end of the basket or cage. In another representative embodiment, the plurality of struts are coupled to a strut ring at a first end of the basket or cage and the plurality of struts are coupled to the distal end of the inner catheter manipulation shaft at a second end of the basket or cage. In a representative embodiment, the catheter may further comprise: an atraumatic, molded end cap coupled to the strut ring.

In a representative embodiment, the plurality of struts are arranged longitudinally only without crossing each other. In a representative embodiment, the inner catheter manipulation shaft is further moveable longitudinally to extend out of the outer catheter tube. In a representative embodiment, the basket or cage is moveable, in a contracted or compressed state, with the inner catheter manipulation shaft, both longitudinally and rotatably within the outer catheter lumen. In a representative embodiment, the basket or cage is further moveable longitudinally to extend out of the distal end of the outer catheter tube, and when the basket or cage is fully extended out of the distal end of the outer catheter tube, the basket or cage has an expanded state. In a representative embodiment, the basket or cage, in the expanded state, has a diameter from 20.0 mm to 50.0 mm. In a representative embodiment, the plurality of struts comprise eight to twelve struts. In a representative embodiment, each strut of the plurality of struts has a width or diameter from 0.25 mm to 0.7 mm and a length from 55 mm to 104 mm. In a representative embodiment, a maximum gap width between each strut of the plurality of struts, in the expanded state of the basket or cage, from 10.0 to 15.0 mm. In a representative embodiment, the basket or cage comprises one or more materials selected from the group consisting of: nitinol, titanium, stainless steel, a metal, a metallic alloy, carbon fiber, a polymer, and combinations thereof.

In a representative embodiment, a system may further comprise: a crushing or fracturing instrument moveable longitudinally and rotatably within the catheter. In a representative embodiment, the crushing or fracturing instrument is moveable longitudinally and rotatably within the inner catheter lumen. In a representative embodiment, the crushing or fracturing instrument is further moveable longitudinally to extend out of the inner catheter lumen and the outer catheter tube.

In a representative embodiment, the crushing or fracturing instrument comprises: a crushing or fracturing shaft; and a crushing or fracturing tip integrally formed with or couped at a first end to the crushing or fracturing shaft, the crushing or fracturing tip having one or more crushing or fracturing spears at a second, distal end.

A representative method of using the system is also disclosed, comprising: moving the inner catheter manipulation shaft longitudinally to extend out of the outer catheter lumen of the outer catheter tube; using the basket or cage, capturing at least one gallstone or gallstone fragment within an interior of the basket or cage; moving the inner catheter manipulation shaft longitudinally to at least partially retract the basket or cage; and moving the crushing or fracturing instrument longitudinally to extend out of the inner catheter lumen and impact the at least one gallstone or gallstone fragment.

In a representative embodiment, each crushing or fracturing spear, of the one or more crushing or fracturing spears, comprises at least two beveled and chamfered inner edges terminating in a sharp point. In a representative embodiment, each crushing or fracturing spear, of the one or more crushing or fracturing spears, may further comprise an opening, relief or cut-out. In another representative embodiment, the crushing or fracturing tip may be at least partially hollow. In another representative embodiment, each crushing or fracturing spear, of the one or more crushing or fracturing spears, comprises at least two beveled edges.

In another representative embodiment, the crushing or fracturing instrument comprises: a crushing or fracturing shaft; and a crushing or fracturing tip integrally formed with or couped at a first end to the crushing or fracturing shaft, the crushing or fracturing tip having at least one configuration selected from the group consisting of: an angled configuration, a pointed configuration, a sharp-edged or beveled configuration, a bullet configuration, a spherical or “button” configuration, a concave tri-tip configuration, a spear configuration, a concave spear configuration, a core drilling configuration, a fluted configuration, a hollow configuration, and combinations thereof. In a representative embodiment, the crushing or fracturing instrument comprises one or more materials selected from the group consisting of: nitinol, titanium, stainless steel, a metal, a metallic alloy, carbon fiber, a polymer, and combinations thereof.

In another representative embodiment, the catheter may comprise: an outer catheter tube having an outer catheter lumen and an outer catheter tube distal end; an inner catheter manipulation shaft moveable within the outer catheter lumen, the inner catheter manipulation shaft having an inner catheter shaft distal end; and a basket or cage coupled to the distal end of the outer catheter tube and to the distal end of the inner catheter manipulation shaft, the basket or cage comprising a plurality of struts. In a representative embodiment, the catheter may further comprise: a catheter tip cap coupled to the plurality of struts, the catheter tip cap having a tip opening; wherein the inner catheter manipulation shaft further comprises an inner catheter shaft lumen coupled to and in fluid communication with the tip opening to form a catheter distal flush port.

In a representative embodiment, the catheter may further comprise: a catheter tube collar coupled to a first end of the basket or cage and to a distal end of the outer catheter tube, wherein one or more corresponding first ends of the plurality of struts are coupled between the catheter tube collar and the outer catheter tube; and a catheter tip cap coupled to a second end of the basket or cage and to a distal end of the inner catheter manipulation shaft, wherein one or more corresponding second ends of the plurality of struts are coupled between the catheter tip cap and the inner catheter manipulation shaft.

In a representative embodiment, the catheter may further comprise: a catheter tube collar coupled to a first end of the basket or cage and to a distal end of the outer catheter tube, wherein one or more corresponding first ends of the plurality of struts are coupled between the catheter tube collar and the outer catheter tube; and a catheter tip cap coupled to a second end of the basket or cage and to a distal end of the inner catheter manipulation shaft, wherein one or more corresponding second ends of the plurality of struts are coupled between the catheter tip cap and the inner catheter manipulation shaft, the catheter tip cap further comprising a tip opening; wherein the inner catheter manipulation shaft further comprises an inner catheter shaft lumen coupled to and in fluid communication with the tip opening to form a catheter distal flush port.

In another representative embodiment, the catheter may further comprise: a catheter tube collar coupled to the plurality of struts; and a catheter tip cap coupled to the plurality of struts. In a representative embodiment, the catheter may further comprise: a catheter tube collar coupled to the plurality of struts; and a catheter tip cap coupled to the plurality of struts, the catheter tip cap having a tip opening; wherein the inner catheter manipulation shaft further comprises an inner catheter shaft lumen coupled to and in fluid communication with the tip opening to form a catheter distal flush port. In another representative embodiment, the plurality of struts are arranged in a selected crossing pattern to form a mesh having a plurality of pores of differing pore sizes. In a representative embodiment, the plurality of struts are arranged in a selected crossing pattern to form a mesh having comparatively larger pores or openings at a proximal region of the basket or cage and having comparatively smaller pores or openings at a distal region of the basket or cage. In a representative embodiment, the outer catheter tube further comprises: a proximal flush port arranged at the outer catheter tube distal end. In another representative embodiment, the plurality of struts are arranged to form a plurality of grasping hooks. In another representative embodiment, the basket or cage further comprises: a plurality of sharp teeth, points, or wedges, one or more sharp teeth, points, or wedges of the plurality of sharp teeth, points, or wedges coupled to or integrally formed with an interior surface of a corresponding strut of the plurality of struts. In a representative embodiment, the plurality of sharp teeth, points, or wedges have one or more shapes selected from the group consisting of: triangular, square, rectangular, round, arced, and combinations thereof.

In another representative embodiment, the system may further comprise: a crushing or fracturing instrument moveable within the outer catheter tube, the crushing or fracturing instrument terminating distally in a sharp or pointed tip. In another representative embodiment, the system may further comprise: a shaft drive coupled to the crushing or fracturing instrument, the shaft drive selected from the group consisting of: a linear actuator, a compression spring driven action, an extension spring drive, a torsion spring driven, spring driven, constant force spring driven, and combinations thereof. In another representative embodiment, the system may further comprise: a crushing or fracturing instrument moveable within the outer catheter tube, the crushing or fracturing instrument terminating distally in tip having a shape selected from the group consisting of: sharp, pointed, rounded, spear headed, diamond, chamfered, chiseled, bull nosed, bullet head, hollow, drill bit, hole saw, dimpled, screw, helical, fin, and combinations thereof. In another representative embodiment, the system may further comprise: a crushing or fracturing instrument insertable into the access sheath.

In another representative embodiment, the access sheath comprises: a tubular shaft having a first, proximal end and a second, distal end, the tubular shaft having a longitudinal axis and a central lumen arranged longitudinally, the tubular shaft comprising: an inner shaft wall forming the central lumen; an outer shaft wall arranged coaxially with and spaced apart radially from the inner shaft wall to form, in between the inner and outer shaft walls, one or more peripheral lumens; and an inflation lumen arranged between the inner and outer shaft walls; and a proximal hub coupled to the first end of the tubular shaft, the proximal hub comprising: a hub housing having a hub lumen, the hub lumen arranged in fluid communication with the central lumen; a filter assembly removably coupleable to the hub housing, the filter assembly arranged in fluid communication with the central lumen; a first port integrally formed with or coupled to the hub housing, the first port arranged in fluid communication with the one or more peripheral lumens; and a second port integrally formed with or coupled to the hub housing, the second port arranged in fluid communication with the inflation lumen; wherein the aspiration device comprises: a syringe tube body having an interior lumen; an extended distal tip coupled to or integrally formed with the syringe tube body; and a plunger moveable within the interior lumen of the syringe tube body; and wherein the catheter comprises: an outer catheter tube having an outer catheter lumen and an outer catheter tube distal end; an inner catheter manipulation shaft moveable longitudinally and rotatably within the outer catheter lumen, the inner catheter manipulation shaft having an inner catheter lumen and an inner catheter shaft distal end; and a basket or cage coupled to the inner catheter manipulation shaft, the basket or cage comprising a plurality of struts arranged longitudinally.

A method of using the system is also disclosed, with a representative method embodiment comprising: inserting at least one dilator percutaneously to a selected location of a patient gallbladder to form a tract; inserting the access sheath into the tract; attaching the aspiration device to the proximal hub; aspirating one or more gallstones; and removing the aspriration device from the proximal hub.

In a representative embodiment, the method may further comprise: inserting the catheter through the access sheath; expanding the basket or cage; using the expanded basket or cage, capturing one or more gallstones; contracting the basket or cage around the one or more gallstones; and removing the catheter through the access sheath. In a representative embodiment, the method may further comprise: using a crushing or fracturing instrument inserted into the inner catheter lumen, fracturing or crushing the one or more gallstones. In a representative embodiment, the method may further comprise: using the aspiration device, flushing the patient gallbladder.

In another representative embodiment, the system may further comprise: a crushing or fracturing control apparatus; and a crushing or fracturing instrument arranged coaxially within the catheter.

In a representative embodiment, the crushing or fracturing control apparatus may comprise: a housing; a control apparatus shaft coupled to the housing, the control apparatus shaft having a control apparatus shaft lumen configured to hold the catheter and the crushing or fracturing instrument arranged within the catheter; a catheter control assembly arranged within the housing, the catheter control assembly comprising: a catheter control actuator: and a crushing or fracturing pin; a first handle pivotably coupled to the housing; a control linkage lever pivotably coupled to the first handle and pivotably coupled to the catheter control actuator; a crushing or fracturing spring coupled around the crushing or fracturing pin; and a second handle coupled to the crushing or fracturing pin.

In a representative embodiment, the catheter control actuator comprises: a tubular catheter grip linearly aligned with the control apparatus shaft lumen, the tubular catheter grip coupleable around an inner catheter manipulation shaft of the catheter to secure and move the inner catheter manipulation shaft longitudinally within the catheter in response to movement of the first handle and the control linkage lever. In a representative embodiment, the crushing or fracturing pin is linearly aligned with the tubular grip within the housing to engage with the crushing or fracturing instrument. In a representative embodiment, the crushing or fracturing pin is arranged coaxially within the tubular grip within the housing to engage with the crushing or fracturing instrument, the crushing or fracturing instrument arranged coaxially within the inner catheter manipulation shaft.

In a representative embodiment, the second handle is slidable proximally with the crushing or fracturing pin within the housing to compress the crushing or fracturing spring. In a representative embodiment, the crushing or fracturing control apparatus further comprises: a retention clip removably coupled to the second handle to retain the second handle in a proximal position with the compressed crushing or fracturing spring. In a representative embodiment, the retention clip is removable to release the compressed crushing or fracturing spring and advance the crushing or fracturing pin to impact the crushing or fracturing instrument.

Another method of using the system is also disclosed, with a representative method embodiment comprising: inserting a catheter and a crushing or fracturing instrument into the control apparatus shaft, the crushing or fracturing instrument arranged coaxially within an inner catheter manipulation shaft of the catheter; securing a proximal end of the catheter in the catheter control actuator; with the first handle in a first position, inserting a distal end of the control apparatus shaft into a gallbladder of a human or veterinary subject; moving the first handle to a second position to advance the inner catheter manipulation shaft and expand a basket or cage at a distal end of the catheter; capturing a gallstone or a gallstone fragment in the basket or cage; and moving the first handle toward the first position to retract the basket or cage and secure the captured gallstone or a gallstone fragment in the basket or cage.

In a representative embodiment, the method may further comprise:
when the captured gallstone or a gallstone fragment is smaller than a predetermined size, withdrawing the control apparatus shaft from the gallbladder of the human or veterinary subject. In a representative embodiment, when the captured gallstone or a gallstone fragment is not smaller than the predetermined size, pulling or sliding the second handle proximally to compress the crushing or fracturing spring; and releasing the second handle to advance the crushing or fracturing pin to impact the crushing or fracturing instrument and move the crushing or fracturing instrument into the captured gallstone or a gallstone fragment to crush or fracture the captured gallstone or a gallstone fragment.

In a representative embodiment, the method may further comprise: using the aspiration device, aspirating any crushed or fractured gallstone fragments. In a representative embodiment, the step of inserting the distal end of control apparatus shaft into the gallbladder of the human or veterinary subject may further comprise: inserting the access sheath into the gallbladder of the human or veterinary subject; inserting the distal end of the control apparatus shaft into the access sheath.

In another representative embodiment, the crushing or fracturing control apparatus may comprise: a housing having a housing slot; a control apparatus shaft coupled to the housing, the control apparatus shaft having a control apparatus shaft lumen configured to hold the catheter and the crushing or fracturing instrument arranged within the catheter; a catheter control assembly arranged within the housing, the catheter control assembly comprising: a catheter control linkage; and a crushing or fracturing pin; a first handle coupled to the catheter control linkage and slidable within the housing slot; a second handle coupled to the crushing or fracturing pin; and a trigger actuator pivotably coupled to the housing and further coupled to engage the catheter control linkage.

In a representative embodiment, the catheter control linkage is linearly aligned with the control apparatus shaft lumen and coupleable to an inner catheter manipulation shaft of the catheter to secure and move the inner catheter manipulation shaft longitudinally within the catheter. In a representative embodiment, the crushing or fracturing pin is linearly aligned with the catheter control linkage within the housing to engage with the crushing or fracturing instrument. In a representative embodiment, the crushing or fracturing pin arranged is coaxial within a lumen of the catheter control linkage within the housing to engage with the crushing or fracturing instrument, the crushing or fracturing instrument arranged coaxially within the inner catheter manipulation shaft. In a representative embodiment, the second handle is slidable proximally with the crushing or fracturing pin within the housing to impact the crushing or fracturing instrument. In a representative embodiment, the trigger actuator is arranged to slideably move the catheter control linkage proximally to retract a basket or cage of the catheter toward the crushing or fracturing instrument.

Another method of using the system is also disclosed, with a representative method embodiment comprising: inserting a catheter and a crushing or fracturing instrument into the control apparatus shaft, the crushing or fracturing instrument arranged coaxially within an inner catheter manipulation shaft of the catheter; securing a proximal end of the catheter in the catheter control linkage; with the first handle in a first position, inserting a distal end of the control apparatus shaft into a gallbladder of a human or veterinary subject; moving the first handle to a second position to advance the inner catheter manipulation shaft and expand a basket or cage at a distal end of the catheter; capturing a gallstone or a gallstone fragment in the basket or cage; and moving the first handle toward the first position to retract the basket or cage and secure the captured gallstone or a gallstone fragment in the basket or cage.

In a representative embodiment, the method may further comprise: when the captured gallstone or a gallstone fragment is smaller than a predetermined size, withdrawing the control apparatus shaft from the gallbladder of the human or veterinary subject. In a representative embodiment, the method may further comprise: when the captured gallstone or a gallstone fragment is not smaller than the predetermined size, sliding the second handle distally to advance the crushing or fracturing pin to impact the crushing or fracturing instrument and move the crushing or fracturing instrument into the captured gallstone or a gallstone fragment to crush or fracture the captured gallstone or a gallstone fragment. In a representative embodiment, the method may further comprise: pressing the trigger actuator to slideably move the catheter control linkage proximally to retract the basket or cage of the catheter toward the crushing or fracturing instrument.

In another representative embodiment, the access sheath comprises: a tubular shaft having a first end and a second end, the tubular shaft having a shaft lumen; and a proximal hub coupled to the first end of the tubular shaft. In a representative embodiment, the proximal hub includes a valve or adapter. In a representative embodiment, the second end of the tubular shaft may be angled or beveled. In a representative embodiment, the shaft lumen has an inner diameter between 5 mm to 12 mm, with the access sheath having a length between 5 cm and 40 cm. The access sheath also may further comprise: a handle coupled to the proximal hub.

The representative system embodiment may also include a balloon dilator expandable to increasing, successively larger diameters, or a plurality of dilators having a corresponding plurality of successively larger diameters, with the largest diameter of the successively larger diameters being equivalent or smaller than an interior diameter of the shaft lumen. The representative system embodiment may also include a balloon-tipped catheter which may overly dilate the access tract to a diameter greater than the outer diameter of the tubular shaft of the access sheath, followed by partial deflation of the balloon-tipped catheter and insertion of the access sheath over the balloon-tipped catheter, which is then further deflated and removed from the access sheath.

In a representative embodiment, the proximal hub may further comprise: one or more access ports integrally formed with the housing and arranged distally to the first valve or distally to the first valve, the one or more access ports in fluid communication with the hub lumen; and a central, in-line access port in fluid communication with the hub lumen. The proximal hub may also further comprise one or more additional (second, third, fourth, etc.) valves arranged within or coupleable to the one or more access ports, with the one or more additional valves or adapters comprising, for example, at least one valve selected from the group consisting of: a duckbill valve; a cross-slit valve; a stopcock valve, a gate valve, a rotary valve; a ball valve; a luer valve; a Tuohy-Borst adapter; and combinations thereof.

The representative system embodiment may also include tubing insertable into the one or more access ports or the central, in-line access port; and a peristaltic pump coupled to the tubing or aspiration device.

In a representative embodiment, the proximal hub may further comprise a first coupling, and in such a representative embodiment, the aspiration device may comprise a second, mating coupling removably coupleable to the first coupling. For example and without limitation, in a representative embodiment the first coupling and the second, mating coupling may be mating quick connect fittings or couplings. For example, the proximal hub may further comprise a first coupling comprising a connector having a flexible ring or gasket, and the aspiration device may further comprise a second, mating coupling removably coupled to the first coupling, the second, mating coupling comprising a recess for insertion of the flexible ring or gasket. In another representative embodiment, the proximal hub may further comprise a first coupling comprising a first tab recess and a second tab recess. In such a representative embodiment, the aspiration device may comprise a second, mating coupling removably coupled to the first coupling, the second, mating coupling comprising a first tab or detent insertable into the first tab recess and a second tab or detent insertable into the second tab recess.

A method of using the system is also disclosed, with the method comprising: inserting at least one dilator percutaneously to a selected location of a patient gallbladder to form a tract; inserting the access sheath into the tract; attaching the aspiration device to the proximal hub; aspirating one or more gallstones; and removing the aspriration device from the proximal hub. For example and without limitation, a series of dilators may be inserted percutaneously to a selected location of a patient gallbladder to form the tract, followed by inserting the access sheath percutaneously to the selected location of a patient gallbladder.

The dilator may then be removed, such as through the proximal portion of the access sheath. In another representative embodiment, also for example and without limitation, the method of using the system comprises inserting a dilation balloon so that the distal end of the balloon (or balloon tip) is inside the gallbladder (forming a tract to the gallbladder) and the proximal end of the balloon is outside the patient; inflating the balloon to dilate the tract; and inserting or advancing the access sheath around the dilation balloon. In addition, the dilation balloon may be partially deflated while the access sheath is being advanced or inserted. The representative method embodiment may also include, also for example and without limitation, inserting a balloon-tipped catheter and dilating the access tract, followed by partial deflation of the balloon-tipped catheter and insertion of the access sheath over the balloon-tipped catheter, which is then further deflated and removed from the access sheath. One or more gallstones are then aspirated.

In a representative embodiment, the method may further comprise: inserting the catheter through the access sheath; expanding the basket or cage; using the expanded basket or cage, capturing one or more gallstones; contracting the basket or cage around the one or more gallstones; and removing the catheter through the access sheath. In such a representative embodiment, the method may further comprise: using the expanded basket or cage, fracturing or crushing the one or more gallstones, including through the use of a second, pointed shaft. In such a representative embodiment, the method may further comprise: using the aspiration device or the catheter, flushing the patient gallbladder.

Numerous other advantages and features of the present invention will become readily apparent from the following detailed description of the invention and the embodiments thereof, from the claims and from the accompanying drawings.

### BRIEF DESCRIPTION OF THE DRAWINGS

The objects, features and advantages of the present invention will be more readily appreciated upon reference to the following disclosure when considered in conjunction with the accompanying drawings, wherein like reference numerals are used to identify identical components in the various views, and wherein reference numerals with alphabetic characters are utilized to identify additional types, instantiations or variations of a selected component embodiment in the various views, in which:

FIG. 1 is a side (elevational) and cut-away view illustrating a representative first embodiment of an access sheath.

FIG. 2 is a partial cut-away view illustrating a tubular shaft of the representative first embodiment of the access sheath inserted around a representative dilator.

FIG. 3 is a partial cross-sectional view (through the C-C′ plane of FIG. 1) illustrating a representative embodiment of a proximal hub having a valve and port assembly, of the first embodiment of the access sheath.

FIG. 4 is an isometric view illustrating a representative first embodiment of an aspiration device, illustrated with a translucent syringe tube body.

FIG. 5 is a partial cross-sectional view illustrating a representative first embodiment of a system having a first embodiment of the access sheath and a representative first embodiment of the aspiration device.

FIG. 6 is a partial isometric view illustrating a representative second embodiment of a system having a second embodiment of an access sheath and a representative first embodiment of the aspiration device, also illustrated as having a translucent syringe tube body.

FIG. 7 is a side (elevational) view illustrating a representative second embodiment of an aspiration device, also illustrated as having a translucent syringe tube body.

FIG. 8 is a side (elevational) view illustrating a representative embodiment of a plunger of an aspiration device.

FIG. 9 is an isometric view illustrating a representative third embodiment of an aspiration device, also illustrated as having a translucent syringe tube body.

FIG. 10 is an isometric view illustrating a representative fourth embodiment of an aspiration device, also illustrated as having a translucent syringe tube body.

FIG. 10A is an isometric view illustrating a representative third embodiment of a system having a third embodiment of an access sheath and a representative fifth embodiment of the aspiration device, also illustrated as having a translucent syringe tube body.

FIG. 11 is an isometric view illustrating representative first and second embodiments of a catheter apparatus having a representative first embodiment of a basket or cage, illustrated in an expanded state.

FIG. 12 is an isometric view illustrating the representative first and second embodiments of the catheter apparatus having the representative first embodiment of the basket or cage, illustrated in the expanded state.

FIG. 13 is a partial cross-sectional view illustrating the representative first embodiment of a catheter apparatus having the representative first embodiment of the basket or cage, illustrated in the expanded state.

FIG. 14 is a partial cross-sectional view illustrating the representative second embodiment of the catheter apparatus having the representative first embodiment of the basket or cage, illustrated in the expanded state.

FIG. 15 is an isometric view illustrating a representative third embodiment of a catheter apparatus having a representative second embodiment of a basket or cage, and illustrated in an expanded state.

FIG. 16 is an isometric view illustrating a representative fourth embodiment of a catheter apparatus having a representative third embodiment of a basket or cage, illustrated in an expanded state.

FIG. 17 is an isometric view illustrating the representative fourth embodiment of the catheter apparatus having the representative third embodiment of a basket or cage, illustrated in an expanded state.

FIG. 18 is an isometric view illustrating the representative fourth embodiment of the catheter apparatus having the representative third embodiment of a basket or cage, illustrated in a contracted state.

FIG. 19 is an isometric view illustrating the representative fourth embodiment of the catheter apparatus having the representative third embodiment of a basket or cage, illustrated in the contracted state.

FIG. 20 is a partial cross-sectional view (through the C-C′ plane of FIG. 1) illustrating a representative fourth embodiment of a system having the first embodiment of the access sheath, additionally with a representative first or second embodiment of an aspiration device, and with a representative catheter embodiment.

FIG. 21 is a partial cross-sectional view (through the C-C′ plane of FIG. 1) illustrating a representative fifth embodiment of a system having a fourth embodiment of an access sheath, additionally with a representative catheter embodiment.

FIG. 22 is a partial cross-sectional view (through the C-C′ plane of FIG. 1) illustrating a representative sixth embodiment of a system having a first embodiment of the access sheath illustrated in FIG. 1, additionally with a representative catheter embodiment.

FIG. 23 is an isometric view illustrating a representative first crushing or fracturing instrument, for use as part of a system with an access sheath.

FIG. 24 is a cut-away view illustrating the representative first crushing or fracturing instrument illustrated in FIG. 23.

FIG. 25A is an isometric view illustrating a representative fifth embodiment of a catheter apparatus having a representative fourth embodiment of a basket or cage having representative sharp teeth, points, or wedges coupled to or integrally formed with one or more interior surfaces of one or more struts, illustrated in an expanded state.

FIG. 25B is a cross-sectional view, through the B-B′ plane of the representative fourth embodiment of the basket or cage illustrated in FIG. 25A, illustrating the cage or basket having representative sharp teeth, points, or wedges coupled to or integrally formed with one or more interior surfaces of one or more struts, illustrated in the expanded state.

FIG. 26 is a cross-sectional view illustrating the representative fourth embodiment of the basket or cage having sharp teeth, points, or wedges coupled to or integrally formed with one or more interior surfaces of one or more struts, illustrated in a contracted state.

FIGS. 27A, 27B, 27C, 27D, and 27E are cross-sectional views illustrating representative configurations of sharp teeth, points, or wedges coupled to or integrally formed with one or more interior surfaces of struts of any of the representative baskets or cages of any of the various representative catheter embodiments, illustrated in contracted states.

FIGS. 28A, 28B, and 28C are isometric views illustrating a fifth embodiment of a basket or cage having grasping hooks for use in or with any of the various representative catheter embodiments, and illustrated in an expanded state.

FIG. 29 is an isometric view illustrating a representative sixth embodiment of a catheter apparatus having a sixth embodiment of a basket or cage, illustrated in the expanded state, with the sixth embodiment of the basket or cage having a proximal hoop design (wire or laser cut) with an attached, braided collection basket.

FIGS. 30A and 30B are isometric and partial cross-sectional views illustrating the capture and initial cracking or crushing of a gallstone using a representative seventh embodiment of a catheter apparatus having a seventh embodiment of a basket or cage, respectively illustrated in an expanded state in FIG. 30A and a partially contracted state in FIG. 30B.

FIG. 31 is an isometric view illustrating a representative second embodiment of a crushing or fracturing instrument.

FIG. 32 is a side, elevational view illustrating a representative fifth embodiment of an access sheath with proximal fixed aspiration from a flexible, self-expanding member.

FIG. 33 is a partial isometric view illustrating a representative third embodiment of a crushing or fracturing instrument.

FIG. 34 is a partial, side, elevational view illustrating the representative third embodiment of the crushing or fracturing instrument.

FIG. 35 is a partial isometric view illustrating a representative fourth embodiment of a crushing or fracturing instrument.

FIG. 36 is a partial, plan view illustrating the representative fourth embodiment of the crushing or fracturing instrument.

FIG. 37 is a partial isometric view illustrating a representative fifth embodiment of a crushing or fracturing instrument.

FIG. 38 is a partial, plan view illustrating the representative fifth embodiment of the crushing or fracturing instrument.

FIG. 39 is a partial, isometric view illustrating a representative sixth embodiment of a crushing or fracturing instrument.

FIG. 40 is a partial, isometric view illustrating a representative seventh embodiment of a crushing or fracturing instrument.

FIG. 41 is a partial, isometric view illustrating a representative eighth embodiment of a crushing or fracturing instrument.

FIG. 42 is a partial, isometric view illustrating a representative ninth embodiment of a crushing or fracturing instrument.

FIG. 43 is a partial, isometric view illustrating a representative tenth embodiment of a crushing or fracturing instrument.

FIG. 44 is a partial, isometric view illustrating a representative eleventh embodiment of a crushing or fracturing instrument.

FIG. 45 is a partial, isometric view illustrating a representative twelfth embodiment of a crushing or fracturing instrument.

FIG. 46 is a partial, isometric view illustrating a representative thirteenth embodiment of a crushing or fracturing instrument.

FIG. 47 is a partial, isometric view illustrating a representative fourteenth embodiment of a crushing or fracturing instrument.

FIG. 48 is a partial, isometric view illustrating a representative fifteenth embodiment of a crushing or fracturing instrument.

FIG. 49 is a partial, isometric view illustrating a representative sixteenth embodiment of a crushing or fracturing instrument.

FIG. 50 is a partial, isometric view illustrating a representative seventeenth embodiment of a crushing or fracturing instrument.

FIG. 51 is a partial, isometric view illustrating a representative eighteenth embodiment of a crushing or fracturing instrument.

FIG. 52 is a partial isometric view illustrating a representative eighth embodiment of a catheter apparatus having an eighth embodiment of a basket or cage.

FIG. 53 is a cross-sectional view (through the F-F′ plane of FIG. 52) illustrating the catheter shaft and catheter shaft lumens of the representative eighth embodiment of the catheter apparatus.

FIG. 54 is a partial isometric view illustrating a representative ninth embodiment of a catheter apparatus having a ninth embodiment of a basket or cage, and illustrating a crushing or fracturing tip of a crushing or fracturing instrument.

FIG. 55 is a partial isometric view illustrating the representative ninth embodiment of a catheter apparatus having a partially retracted ninth embodiment of a basket or cage.

FIG. 56 is an isometric view illustrating a representative tenth embodiment of a basket or cage.

FIG. 57 is an isometric view illustrating a representative eleventh embodiment of a basket or cage.

FIG. 58 is an isometric, partially exploded view illustrating a representative seventh embodiment of a system having representative fifth and sixth embodiments of an access sheath and any of the representative embodiments of an aspiration device (also illustrated as having a translucent syringe tube body).

FIG. 59 is a partial, isometric and cut-away view illustrating the representative seventh embodiment of the system having the representative fifth and sixth embodiments of the access sheath and any of the representative embodiments of an aspiration device.

FIG. 60 is a first cross-sectional view (through the E-E′ plane of FIG. 58) illustrating a first tubular shaft of the representative fifth embodiment of the access sheath of the representative seventh embodiment of the system.

FIG. 61 is a second cross-sectional view (through the E-E′ plane of FIG. 58) illustrating a second tubular shaft of the representative sixth embodiment of the access sheath of the representative seventh embodiment of the system.

FIG. 62 is a partial cross-sectional view (through the D-D′ plane of FIGS. 58 and 70) illustrating the proximal hub of the representative fifth and sixth embodiments of the access sheath of the representative seventh and eighth embodiments of the system.

FIG. 63 is a partial cross-sectional view (through the D-D′ plane of FIGS. 58 and 70) illustrating the representative fifth embodiment of the proximal hub and access sheath of the representative seventh and eighth embodiments of the system, additionally with a representative catheter embodiment.

FIG. 64 is a cut-away view illustrating a representative first embodiment of an access sheath tip.

FIG. 65 is a cut-away view illustrating a representative second embodiment of an access sheath tip.

FIG. 66 is a cut-away view illustrating a representative third embodiment of an access sheath tip.

FIG. 67 is a cut-away view illustrating a representative fourth embodiment of an access sheath tip.

FIG. 68 is a partial, isometric view illustrating a representative fifth embodiment of an access sheath tip and a sealing and anchoring balloon of any of the various embodiments of an access sheath.

FIG. 69 is a partial, isometric, cut-away view illustrating a representative sixth embodiment of an access sheath tip.

FIG. 70 is an isometric view illustrating a representative eighth embodiment of a system having seventh and eighth embodiments of an access sheath and a first embodiment of a dual aspiration and infusion assembly.

FIG. 71 is a partial, isometric view illustrating a representative proximal hub of the representative eighth system embodiment having the seventh and eighth access sheath embodiments.

FIG. 72 is a diagram illustrating fluid flow in a representative gallbladder using the seventh and/or eighth system embodiments.

FIG. 73 is an isometric view illustrating a representative second embodiment of a dual aspiration and infusion assembly which may be utilized as part of the representative eighth system embodiment.

FIG. 74 is an isometric view illustrating the operation of the first embodiment of a dual aspiration and infusion assembly.

FIG. 75 is a top, plan view illustrating the operation of the first embodiment of a dual aspiration and infusion assembly.

FIG. 76 is a cut-away, top plan view illustrating a representative gearing assembly for operation of the first embodiment of a dual aspiration and infusion assembly.

FIG. 77 is a cut-away, top plan view illustrating operation of the representative gearing assembly for operation of the first embodiment of a dual aspiration and infusion assembly.

FIG. 78 is a cut-away, top plan view illustrating operation of the representative gearing assembly for operation of the first embodiment of a dual aspiration and infusion assembly.

FIG. 79 is an isometric view illustrating a representative ninth embodiment of an access sheath.

FIG. 80 is a cut-away view illustrating the representative ninth embodiment of an access sheath of FIG. 79.

FIG. 81 is a partial cross-sectional view (through the G-G′ plane of FIG. 79) illustrating the representative ninth embodiment of the access sheath, additionally with a representative catheter embodiment.

FIG. 82 is an isometric view illustrating a representative ninth embodiment of a system having a ninth embodiment of the access sheath and a first embodiment of a crushing or fracturing control apparatus, with a ninth embodiment of a catheter apparatus having a ninth embodiment of a basket or cage and any of the representative embodiments of an aspiration device.

FIG. 83 is an isometric view illustrating a representative tenth embodiment of a system having the ninth embodiment of the access sheath and a second embodiment of a crushing or fracturing control apparatus, with a ninth embodiment of a catheter apparatus having a ninth embodiment of a basket or cage and any of the representative embodiments of an aspiration device.

FIG. 84 is a side, elevational and partial cut-away view illustrating a representative two-part tenth embodiment of an access sheath.

FIG. 85 is a side, elevational and partial cut-away view illustrating the representative two-part tenth embodiment of an access sheath.

FIG. 86 is a partial, first cross-sectional view (through the H-H′ plane of FIG. 84) illustrating a seventh embodiment of an access sheath tip of the representative tenth embodiment of an access sheath.

FIG. 87 is a partial, second cross-sectional view (through the H-H′ plane of FIG. 84) illustrating a portion of a proximal hub of the representative tenth embodiment of an access sheath.

FIG. 88 is a side, elevational and partial cut-away view illustrating a first removably coupleable part of the representative tenth embodiment of an access sheath.

FIG. 89 is a side, elevational and partial cut-away view illustrating a second removably coupleable part of the representative tenth embodiment of an access sheath.

FIG. 90 is a first isometric view illustrating a representative first embodiment of a crushing or fracturing control apparatus with a representative catheter embodiment.

FIG. 91 is a second isometric view illustrating the representative first embodiment of a crushing or fracturing control apparatus.

FIG. 92 is a partial cut-away view illustrating the representative first embodiment of a crushing or fracturing control apparatus with a proximal portion of a representative catheter embodiment of FIG. 90.

FIG. 93 is a first isometric view illustrating a representative second embodiment of a crushing or fracturing control apparatus with a representative catheter embodiment.

FIG. 94 is a second isometric view illustrating the representative second embodiment of a crushing or fracturing control apparatus.

FIG. 95 is a first partial cut-away view illustrating the representative second embodiment of a crushing or fracturing control apparatus with a proximal portion of a representative catheter embodiment of FIG. 93.

FIG. 96 is a second partial cut-away view illustrating the representative second embodiment of a crushing or fracturing control apparatus.

FIG. 97 is a third partial cut-away view illustrating the representative second embodiment of a crushing or fracturing control apparatus.

FIG. 98 is front, elevational view illustrating a representative peristaltic pump for use as part of a system.

FIGS. 99A and 99B, collectively referred to as FIG. 99, is a flow chart illustrating representative methods for percutaneous removal of gallstones for the treatment of cholelithiasis.

### DETAILED DESCRIPTION OF REPRESENTATIVE EMBODIMENTS

While the present invention is susceptible of embodiment in many different forms, there are shown in the drawings and will be described herein in detail specific exemplary embodiments thereof, with the understanding that the present disclosure is to be considered as an exemplification of the principles of the invention and is not intended to limit the invention to the specific embodiments illustrated. In this respect, before explaining at least one embodiment consistent with the present invention in detail, it is to be understood that the invention is not limited in its application to the details of construction and to the arrangements of components set forth above and below, illustrated in the drawings, or as described in the examples. Methods and apparatuses consistent with the present invention are capable of other embodiments and of being practiced and carried out in various ways. Also, it is to be understood that the phraseology and terminology employed herein, as well as the abstract included below, are for the purposes of description and should not be regarded as limiting.

As mentioned above, various representative embodiments provide apparatuses, methods and systems specifically designed for the percutaneous removal of gallstones for the treatment of cholelithiasis. The representative embodiments are capable of percutaneous and transhepatic placement into an affected gallbladder, are capable of crushing or fracturing gallstones, and are capable of capturing and removing any gallstones and gallstone fragments from the gallbladder. The representative apparatuses, methods and systems are comparatively low cost, particularly in comparison to costs associated with the surgical treatment of cholelithiasis and subsequent inpatient hospitalization. The representative apparatuses, methods and systems are capable of operation in a typical interventional radiology suite, rather than a surgical operating room. The representative embodiments further provide a treatment option for patients who are not surgical candidates, and thereby improve the quality of life which would otherwise not be available to such patients.

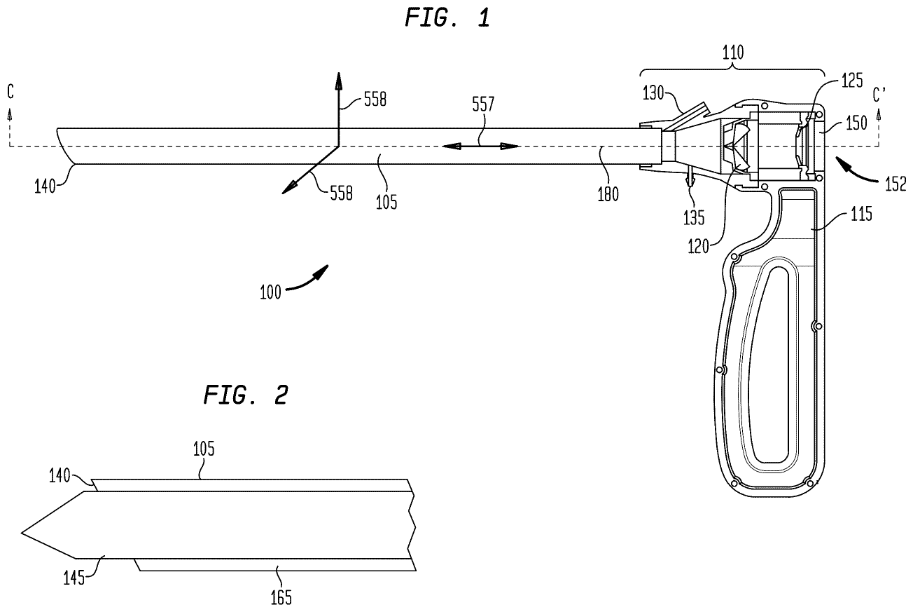

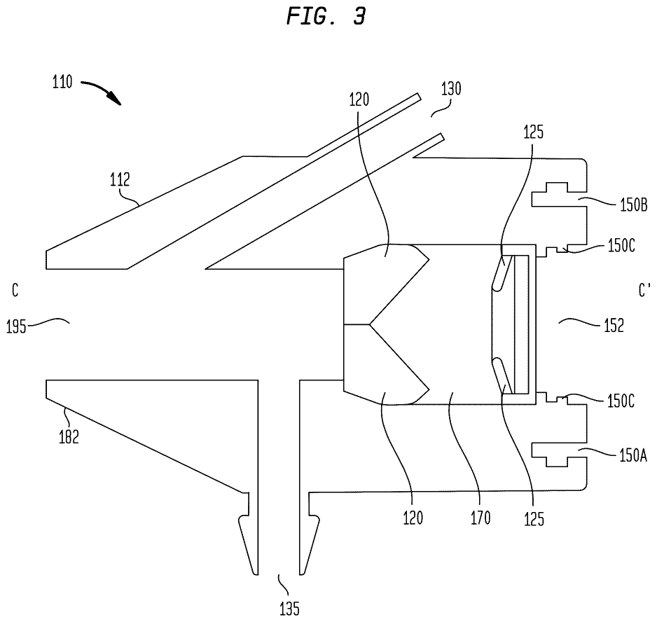

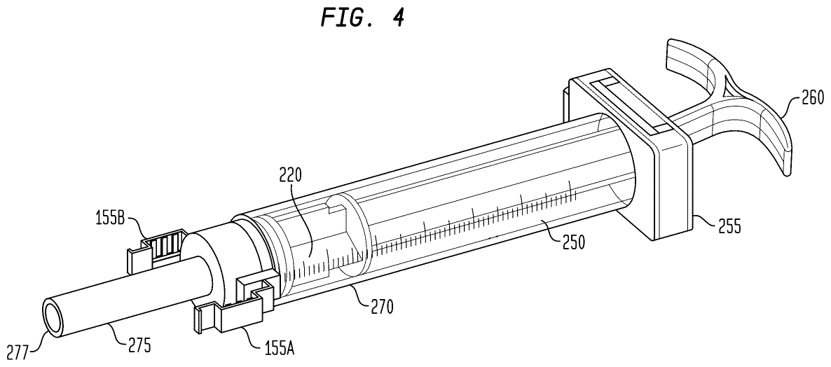

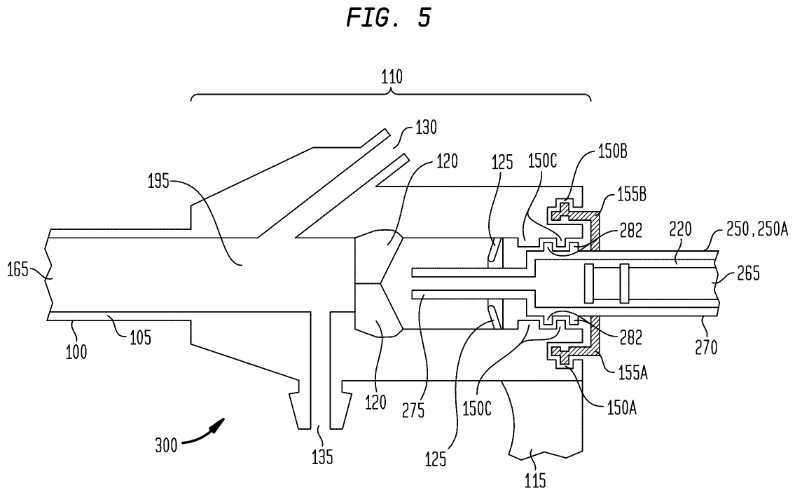

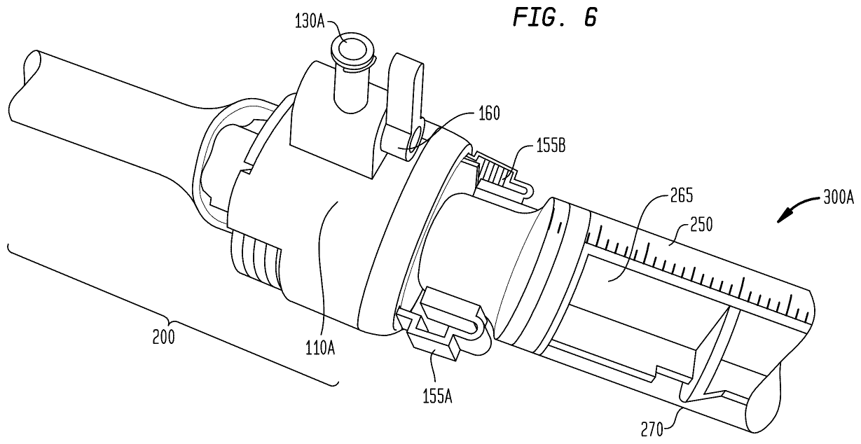

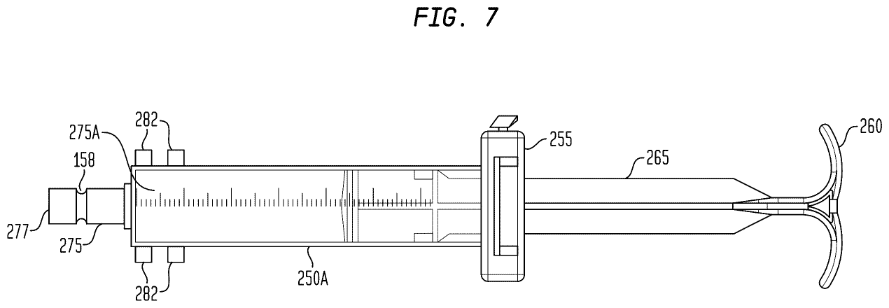

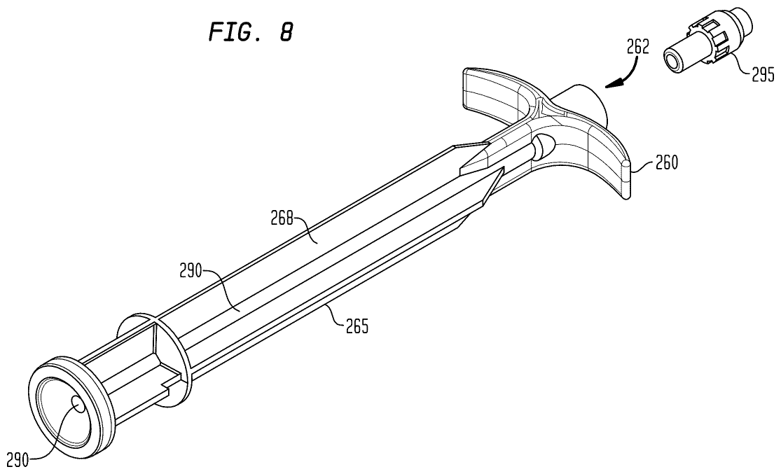

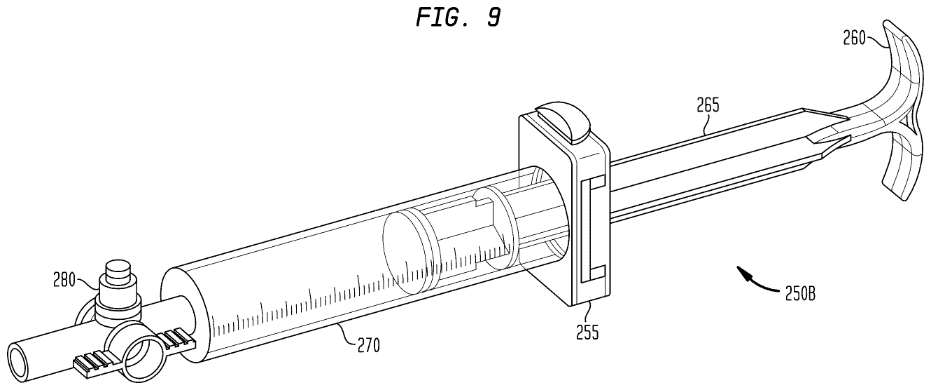

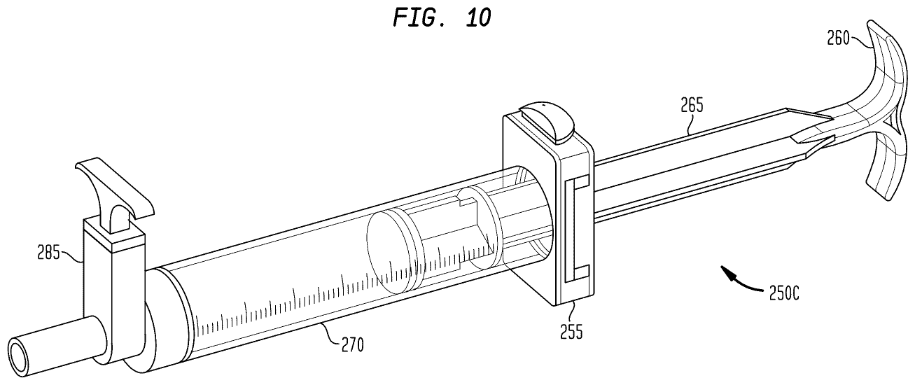

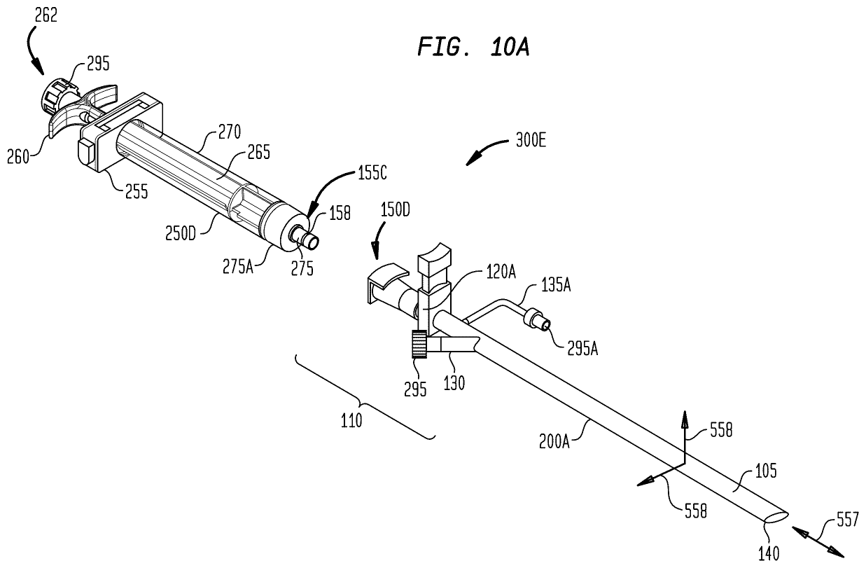

FIG. 1 is a side (elevational) and cut-away view illustrating a representative first embodiment of an access sheath 100. FIG. 2 is a partial cut-away view illustrating a tubular shaft 105, of the representative first embodiment of the access sheath 100 illustrated in FIG. 1, inserted around a representative dilator 145, which may be a generally cylindrical dilator having a conical tip as illustrated, or which may be a balloon dilator or balloon-tipped catheter, with any and all such dilator 145 variations being considered equivalent and within the scope of the disclosure. It should also be noted that the various access sheaths described herein may also be inserted percutaneously into a subject gallbladder through a pre-existing (or mature) tissue tract, also for example and without limitation. FIG. 3 is a partial cross-sectional view (through the C-C′ plane of FIG. 1) illustrating a representative embodiment of a proximal hub 110 having a valve and port assembly of the first embodiment of the access sheath 100 illustrated in FIG. 1. FIG. 4 is an isometric view illustrating a representative first embodiment of an aspiration device 250, illustrated with a translucent syringe tube body 270 to also view a plunger 265 moveable within the syringe tube body 270. FIG. 5 is a partial cross-sectional view illustrating a representative first embodiment of a system 300 having a first embodiment of the access sheath 100 and a representative first embodiment of the aspiration device 250. FIG. 6 is an isometric view illustrating a representative second embodiment of a system 300A having a second embodiment of an access sheath 200 and a representative first embodiment of the aspiration device 250, also illustrated as having a translucent syringe tube body 270. FIG. 7 is a side (elevational) view illustrating a representative second embodiment of an aspiration device 250A, also illustrated as having a translucent syringe tube body 270 to also view a plunger 265 moveable within the syringe tube body 270. FIG. 8 is a side (elevational) view illustrating a representative embodiment of a plunger 265 of an aspiration device 250. FIG. 9 is an isometric view illustrating a representative third embodiment of an aspiration device 250B, also illustrated as having a translucent syringe tube body 270 to also view a plunger 265 moveable within the syringe tube body 270. FIG. 10 is an isometric view illustrating a representative fourth embodiment of an aspiration device 250C, also illustrated as having a translucent syringe tube body 270 to also view a plunger 265 moveable within the syringe tube body 270. FIG. 10A is an isometric view illustrating a representative third embodiment of a system 300E having a third embodiment of an access sheath 200A and a representative fifth embodiment of the aspiration device 250D, also illustrated as having a translucent syringe tube body 270 to also view a plunger 265 moveable within the syringe tube body 270.

Numerous systems, access sheaths, aspiration devices, catheters, catheter baskets or cages, and crushing or fracturing instruments, are described in detail herein. Unless the context clearly dictates otherwise, reference to any system 300-300J shall be understood to mean and include any of the other systems 300-300J. Unless the context clearly dictates otherwise, reference to any access sheath 100, 100A, 200, 200A, 500, 500A, 600, 600A, 800, 900 shall be understood to mean and include any of the other access sheaths 100, 100A, 200, 200A, 500, 500A, 600, 600A, 800, 900. Similarly, unless the context clearly dictates otherwise, reference to any aspiration device 250, 250A, 250B, 250C, 250D shall be understood to mean and include any of the other aspiration devices 250, 250A, 250B, 250C, 250D. Also, unless the context clearly dictates otherwise, reference to any catheter 400 400H shall be understood to mean and include any of the other catheters 400-400H, and reference to any basket or cage 405-405K (equivalently referred to as a cage or basket 405 405K and vice-versa) shall be understood to mean and include any of the other baskets or cages 405-405K. Also, unless the context clearly dictates otherwise, reference to any crushing or fracturing instruments 700-700S shall be understood to mean and include any of the other crushing or fracturing instruments 700-700S. In addition, unless the context clearly dictates otherwise, any of the various systems 300-300J, access sheaths 100, 100A, 200, 200A, 500, 500A, 600, 600A, 800, 900, aspiration devices 250, 250A, 250B, 250C, 250D, catheters 400-400H, and baskets or cages 405-405K, and any of their respective components, may be utilized in any and all combinations with each other, in addition to those illustrated, and any and all such combinations are within the scope of the disclosure.

Referring to FIGS. 1-10A, the access sheath 100 comprises a cylindrical, hollow, tubular shaft or member 105 having an interior shaft lumen 165. The tubular shaft or member 105 having an interior shaft lumen 165, along with the other tubular shafts 505, 505A described below and illustrated in various Figures (with reference to access sheaths 100A, 200, 200A, 500, 500A, 600, 600A, 800, 900), are considered to be oriented in the longitudinal dimension 557, i.e., longitudinally, with the transverse (or radial) dimension 558 being orthogonal to the longitudinal dimension 557, e.g., spaced-apart or offset transversely or radially.

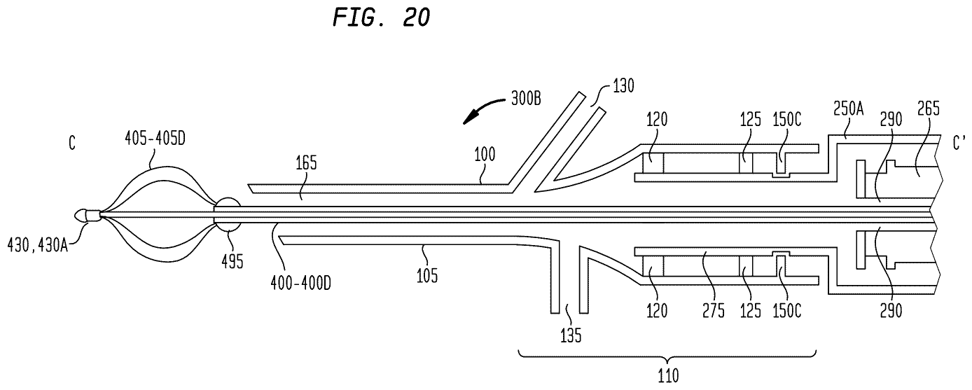

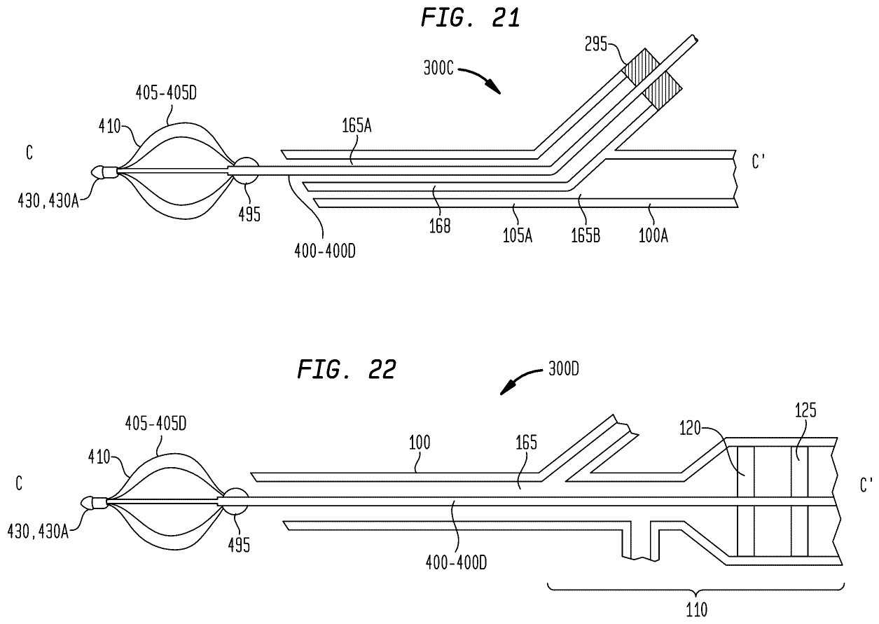

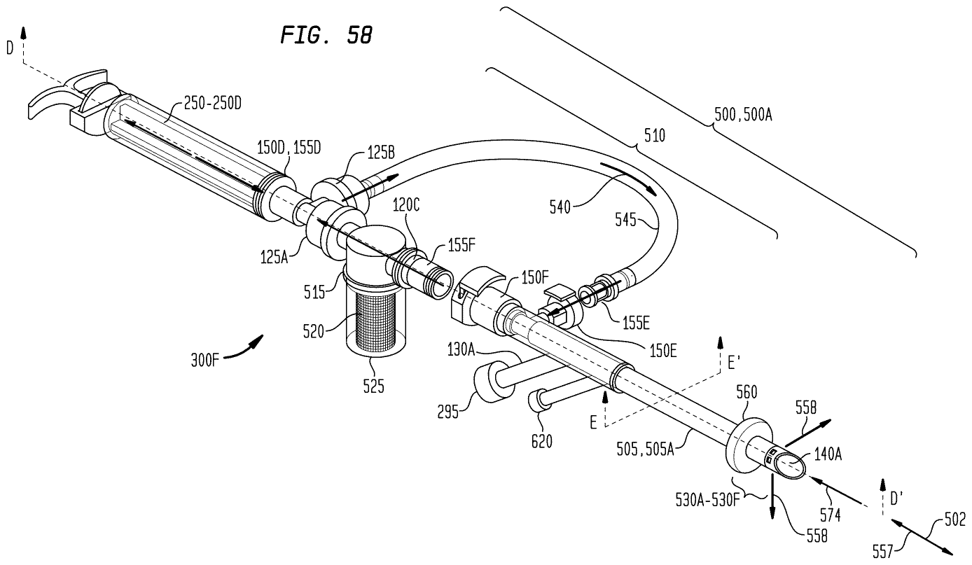

In various embodiments, the tubular shaft 105 may have multiple, separate interior shaft lumens (separated by one or more lumen walls), as illustrated and described below with reference to FIG. 21. Other multi-walled tubular shafts 505, 505A with multiple types of lumens are also illustrated and described below with reference to FIGS. 58-71 and 79-81. A proximal hub 110 is coupled to a first, proximal end 180 of the tubular shaft 105, and further coupled to an optional (first) handle 115. The access sheath tubular shaft or member 105 further has a second, distal end 140, which may be angled or beveled, as illustrated in FIG. 1. The optional handle 115 may be utilized by medical personnel or other users to insert, guide and manipulate the access sheath 100 in a human or veterinary subject or patient, such as for the insertion and positioning of the distal end 140 into the gallbladder of such a patient or subject.

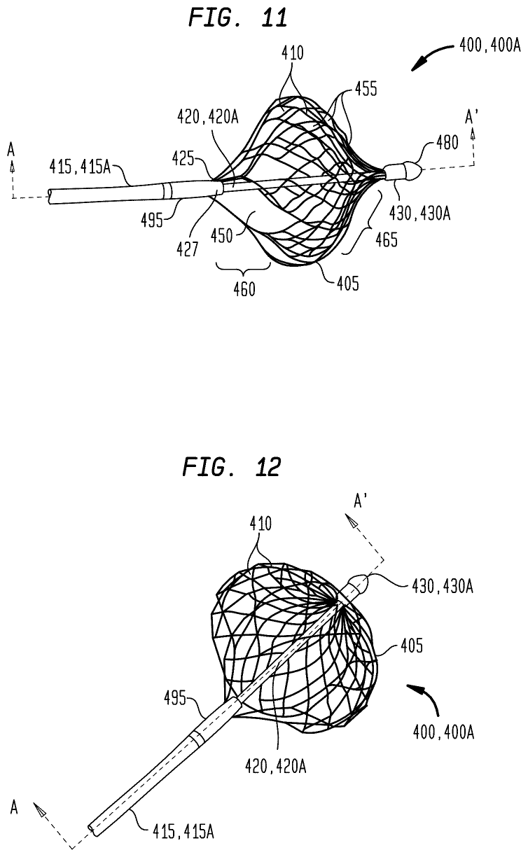

In a representative embodiment, the proximal hub 110 (and 110A) comprises a housing 112 having a hub lumen 195, a first valve 120 arranged in the hub lumen 195; a second valve 125 arranged in the hub lumen and spaced apart proximally from the first valve 120 to provide a hub chamber (e.g., a vacuum chamber) 170 between the first and second valves 120, 125 within the housing 112; one or more access ports 130, 130A, 135 within the housing 112, with the one or more access ports 130, 130A, 135 in fluid communication with the hub lumen 195; and a first coupling 150 (illustrated in FIG. 3 as first and second coupling tab recesses 150A, 150B, respectively), such as to connect to one or more second, corresponding or mating couplings 155 of an aspiration device 250, 250A, 250B, 250C, 250D (e.g., illustrated in FIG. 4 as first and second coupling mating tabs (or detents) 155A, 155B insertable into the corresponding coupling recesses 150A, 150B of the proximal hub 110, or illustrated in FIG. 10A, as first and second quick connect couplings 150D, 155C). The first coupling 150 is typically integrally formed, e.g., injection molded, as part of the housing 112. The housing 112 may have any suitable shape or form factor, in addition to the shape illustrated in cut-away in FIG. 1. The one or more access ports 130, 130A, 135 are generally arranged toward the distal portion 182 of the proximal hub 110, as illustrated, but also may be arranged elsewhere in the proximal hub 110, such as in between the first and second valves 120, 125, for example and without limitation. The hub lumen 195 is generally continuous with and in fluid communication with the shaft lumen 165, as illustrated in FIG. 5. The one or more access ports 130, 130A, 135 provide access to the shaft lumen 165 (via the hub lumen 195) and/or multiple shaft lumens 165A, 165B for the insertion of various medical or surgical devices such as one or more catheters 400, as illustrated and described below, such as with reference to FIGS. 11-22, for the capture and removal of one or more gallstones or gallstone fragments. Not separately illustrated in FIG. 3, the one or more access ports 130, 135 may also include one or more valves, such as to maintain a vacuum within the access sheath 100 and/or the aspiration device 250, such as a valve 160 for access port 130A illustrated and described below with reference to FIG. 6.

Referring to FIG. 6, the access sheath 200 differs from the access sheath 100 insofar as the access sheath 200 includes a proximal hub 110A and does not include a handle 115. The proximal hub 110A differs from the proximal hub 110 insofar as the illustrated access port 130A is provided at a right angle rather than an obtuse angle and further includes a valve 160, illustrated as a stopcock valve, to seal the access port 130A and prevent ingress or egress through the access port 130A. One or more such valves 160 (or any other type of valve) also may be utilized with the one or more access ports 130, 135. In representative embodiments, the proximal hub 110A also includes the other components of the proximal hub 110 as illustrated in FIG. 5, including the first valve 120 and a second valve 125 spaced apart from the first valve 120 to provide a hub chamber (e.g., a vacuum chamber) 170 between the first and second valves 120, 125; and a first coupling 150 (illustrated in FIG. 3 as first and second tab recesses 150A, 150B, respectively), such as to connect to one or more second, corresponding or mating couplings 155 of an aspiration device 250, 250A, 250B, 250C, 250D (250-250D), (illustrated in FIG. 4 as first and second mating tabs (or detents) 155A, 155B insertable into the corresponding recesses 150A, 150B of the proximal hub 110). The mating tabs (or detents) 155A, 155B, as illustrated in FIG. 4, are shown in FIG. 6 being inserted into the first and second coupling tab recesses 150A, 150B of the proximal hub 110A. Other than having these specific differences, the system 300A otherwise has the same components of and functions identically to the system 300.

An aspiration device 250-250D, illustrated as a syringe, for example and without limitation, comprises a hollow, syringe tube body 270 and a plunger 265 moveable within an interior aspiration lumen 220 of the syringe tube body 270. As the plunger 265 is withdrawn through the syringe tube body 270, a vacuum is created within the interior aspiration lumen 220. As described in greater detail below, using various valves or locking devices 255, 280, and/or 285, a vacuum pressure may be maintained within the interior aspiration lumen 220 and, further, may be released suddenly to create an impulse or burst vacuum aspiration.

The aspiration device 250-250D may have an extended, distal tip 275 coupled to or integrally formed with the syringe tube body 270. The distal tip 275 is longer than the tip of a more typical syringe, with the distal tip 275 having sufficient length to be inserted or insertable into the proximal hub 110, 110A, with the first end 277 of the distal tip 275 generally arranged in between the first and second valves 120, 125 in the system 300 300D, in a representative embodiment. Depending upon the selected embodiment, the distal tip 275 of the aspiration device 250-250D may be inserted through both first and second valves 120, 125 of the proximal hub 110, 110A and into the interior lumen 165 of the tubular shaft 105. The aspiration device 250-250D has the second, mating coupling 155, such as mating tabs 155A, 155B or a luer lock or other coupling, to connect or couple to the first coupling 150 (e.g., recesses 150A, 150B) of the proximal hub 110, 110A. The plunger 265 comprises a plunger body 268 coupled to a (second) plunger handle 260, for control and manipulation of the plunger 265, such as for creating a vacuum within the aspiration device 250-250D. In a representative embodiment, as an option, the plunger 265 may also comprise a plunger lumen 290 arranged longitudinally along the plunger body 268 and through the plunger handle 260 and plunger port 262, such as for the insertion of various medical or surgical devices such as one or more catheters 400, in-line or coaxially with the aspiration device 250-250D (and tubular shaft 105), such as illustrated and described below with reference to FIGS. 10A and 20. In a representative embodiment, as an option, the aspiration device 250-250D further comprises one or more (third) valves 255, 280, and/or 285, utilized to create and release an impulse or burst vacuum, described in greater detail below, such as for aspiration of one or more gallstones or gallstone fragments. Also in a representative embodiment, as an option, the aspiration device 250-250D (or access sheath 100) may further comprise an additional (fourth) valve 295, such as a luer activated valve, through which a catheter 400 may be inserted into the lumen 290 of the aspiration device 250-250D or access sheath 100-900, for example and without limitation.

Any of the aspiration devices 250-250D may have any of the various components of any of the other aspiration devices 250-250D, in any combination, and are illustrated separately to point out these various different features which are available and may be included in any selected embodiment. For example and without limitation, aspiration device 250 is illustrated as including mating tabs (or detents) 155A, 155B for insertion into first and second tab recesses 150A, 150B arranged laterally in the proximal hub 110, while aspiration device 250A (and/or 250) may include tabs or threads 282 at the distal tip 275A of the syringe tube body 270, for insertion coaxially into a mating recess, threads or tabs 150C arranged centrally in a proximal hub 110, 110A, forming a luer lock, for example and without limitation. The aspiration device 250B includes a valve 280 (illustrated as stopcock valve) as part of the distal tip 275, while the aspiration device 250C includes a detachable gate valve 285 (which has an interior, sliding valve for sealing) as part of the distal tip 275. As illustrated, the various aspiration devices 250-250D are shown having an optional valve or locking device 255, which can be utilized to lock the plunger 265 in a selected position or otherwise maintain a vacuum within the aspiration device 250-250D. As described in greater detail below, there are many different valves which may be utilized within the aspiration device 250-250D and proximal hub 110, 110A (and other proximal hubs described below), any and all of which are considered equivalent and within the scope of the disclosure. For example and without limitation, a wide variety of valves and locking devices suitable for use herein are commercially available from Inari Medical, Inc. of Irvine, California, US.

The system 300 for gallstone removal comprises several components, including an access sheath 100 and an aspiration device 250. More specifically, in a representative embodiment, a system 300 comprises an access sheath 100 coupled through a proximal hub 110 to an aspiration device 250-250D, such as illustrated in FIG. 5. In another representative embodiment, a system 300A comprises an access sheath 200 coupled through a proximal hub 110A to an aspiration device 250-250D, such as illustrated in FIG. 6. Any of these various systems may further include a catheter 400, arranged coaxially with the access sheath 100 or 200 and aspiration device 250, 250A, 250B, or 250C or arranged through one or more access ports 130, 130A, 135. In another representative embodiment, a system 300B comprises an access sheath 100 or 200 coupled to an aspiration device 250-250D, with a catheter 400 arranged coaxially (or in-line) with the access sheath 100 or 200 and aspiration device 250-250D, such as illustrated in FIG. 20. In another representative embodiment, a system 300C comprises an access sheath 100 or 200 with a catheter 400 arranged through one or more access ports 130, 130A, 135, such as illustrated in FIG. 21, and may further include an aspiration device 250-250D. In another representative embodiment, a system 300D comprises an access sheath 100 or 200 with a catheter 400 arranged coaxially (or in-line) with the access sheath 100 or 200, such as illustrated in FIG. 22, and may further include an aspiration device 250-250D.

A representative third embodiment of a system 300E having a third embodiment of an access sheath 200A and a representative fifth embodiment of the aspiration device 250D are illustrated in FIG. 10A. Referring to FIG. 10A, the access sheath 200 differs from the access sheath 100 insofar as the access sheath 200A includes a proximal hub 110B and also does not include a handle 115. The proximal hub 110B differs from the proximal hub 110 insofar as: (1) the proximal hub 110B includes one (first) valve 120A, illustrated as a large bore gate valve, and does not include a second valve 125; (2) the illustrated first access port 130 includes a (third) valve 295, to seal the access port 130 and prevent ingress or egress through the access port 130, such as for sealing for insertion of a scope or lithotripter; and (3) the second access port 135 is illustrated as a flushing port 135A having a (fourth) valve 295A, such as a luer valve, to seal the access port 135A and prevent ingress or egress through the access port 135A when no flushing is occurring. One or more such valves 295, 295A (or any other type of valve) also may be utilized with the one or more access ports 130, 135. In representative embodiments, the proximal hub 110A also includes a first coupling 150D, as a “quick connect” coupling, such as to connect to a second, corresponding or mating coupling 155C of the aspiration device 250D. Not separately illustrated in FIG. 10A, the first coupling 150D includes a flexible ring or gasket, which seats or mates within the illustrated annular recess 158 of the second coupling 155C. The aspiration device 250D also includes an in-line port 262, also having a valve 295, for insertion of a catheter 400-400H or other instruments, coaxially (in-line) with the aspiration device 250D and access sheath 200A, along with having a gate valve 255. The distal tip 275A of the aspiration device 250D is also removably coupled to the syringe tube body 270, such as through screw threads or a luer lock (not separately illustrated in FIG. 10A). Other than having these specific differences, the system 300E otherwise has the same components of and functions identically to the other systems 300-300D.

In a representative embodiment, the access sheath 100, 100A, 200, 200A, 500, 500A, 600, 600A, 800, 900 has a comparatively large central bore or lumen 165, 555, generally anywhere from 5 mm to 12 mm in inner diameter, with the access sheath 100 generally being 10-20 cm long, but can be as short as 5 cm and as long as 40 cm. In some embodiments, the tubular shaft 105, 505 of the access sheath 100, 100A, 200, 200A, 500, 500A, 600, 600A, 800, 900 is stiff and rigid. In these embodiments the tubular shaft 105, 505 can be either polymeric or metallic, or both, as described in greater detail below. In other embodiments, the tubular shaft 105, 505 of the access sheath 100, 100A, 200, 200A, 500, 500A, 600, 600A, 800, 900 is flexible and can be polymeric, metallic, or both. Regardless of the materials used, the tubular shaft 105, 505 of the access sheath 100, 100A, 200, 200A, 500, 500A, 600, 600A, 800, 900 can be straight, curved, tapered, angled, or deflectable/steerable. The access sheath 100, 100A, 200, 200A includes a proximal hub 110, 110A respectively to provide access for additional tools and scopes, and is supplied with a mating aspiration device 250, 250A, 250B, 250C, 250D such as a syringe to facilitate aspirations. The distal end 140 of the access sheath 100, 100A, 200, 200A, 500, 500A, 600, 600A, 800, 900 may be angled (or beveled, as illustrated), to maximize the size of the gallstone it could remove (or ingest). The distal end 140 could embody a securing mechanism such as a balloon (560) or Nitinol leaflet that would expand to create gallbladder wall apposition in order to keep the access sheath 100, 100A, 200, 200A, 500, 500A, 600, 600A, 800, 900 in place while working through the lumen 165, 555, such as illustrated in FIGS. 58, 59, 68, 69, 70, 71, and 79-88, for example.

A balloon tip could also help guide the access sheath 100 through the tract into the subject gallbladder. Additionally, the system may include one or more tapered dilators 145 to assist with insertion and tracking of the access sheath 100 to the gallbladder. In operation, a series of dilators, a balloon dilator, or balloon-tipped catheter, having increasing diameters are inserted into the patient or subject, to create a tract, with the tubular shaft 105, 505 of the access sheath 100, 100A, 200, 200A, 500, 500A, 600, 600A, 800, 900 inserted into the tract (with the dilator having been removed) and into the subject gallbladder (and/or with the lumen 165, 555 of the tubular shaft 105, 505 of the access sheath 100, 100A, 200, 200A, 500, 500A, 600, 600A, 800, 900 inserted around the dilator 145 and into the subject gallbladder, followed by removal of the dilator(s) 145 from the lumen 165). Alternatively, the tubular shaft 105, 505 of the access sheath 100, 100A, 200, 200A, 500, 500A, 600, 600A, 800, 900 may be inserted into the subject gallbladder through a pre-existing (or mature) tissue tract.

In a representative embodiment, the proximal hub 110, 110A (and other proximal hubs described herein), may contain a single, central, in-line port 152 for utilization for flushing, aspiration, and introduction of tools and auxiliary devices. Additionally, as illustrated in FIGS. 3 and 6, in another representative embodiment, the proximal hub 110, 110A, could contain one or more access ports 130, 130A, 135 for flushing, aspiration, and introduction of tools/auxiliary devices, in addition to or in lieu of an in-line port 152. In some embodiments, these instruments/auxiliary devices can be delivered through shared ports, and in others, they have dedicated ports for increased procedural flexibility. In some embodiments, the tubular shaft 105 could have a single lumen 165 that would connect to the proximal hub 110, 110A. The proximal hub 110, 110A with a single access point or multiple ports, as mentioned above, can all feed into the single lumen 165 of the tubular shaft 105. In another embodiment, the tubular shaft 105, 505 may have multiple lumens (two or more) and can have dedicated lumens for aspiration, tool access, etc., such as illustrated and described below with reference to FIG. 21. Each of these lumens of the tubular shaft 105 can then be individually accessed by each port in the proximal hub 110, 110A. In another embodiment, the accessory port can be incorporated into the aspiration device 250 such that aspiration could be applied simultaneously during use of a camera or mechanical tool. This would involve a lumen 290 through the plunger 265 of the aspiration device 250 with a valve (e.g., valve 295) to seal around the inserted tool.

The proximal hub 110, 110A of the access sheath 100, 100A, 200, 200A could contain a two-stage valve as illustrated in FIG. 3, comprising a first, distal valve 120 and a second, proximal, or backup valve (or seal) 125. The proximal hub 110, 110A may be configured to detachably connect to the aspiration device 250-250D (or syringe), such as using the illustrated mating connectors 150, 155, 282 (such as luer locks, for example and without limitation). The second, proximal valve 125 seals around the distal tip 275 of the aspiration device 250-250D, and the first, distal valve 120 seals on itself, which creates a hub chamber 170 within which vacuum can be generated, such as an impulse or burst vacuum created (a “whoosh” charged). While the vacuum is being generated, the distal tip 275 of the aspiration device 250-250D (or syringe) remains in the hub (vacuum) chamber 170, which is defined by the measurable space or distance between the first, distal valve 120 and the second, proximal valve 125. Generating a vacuum in the hub chamber 170 allows the user to suddenly expose this created or charged vacuum to the target treatment space and create a quick, sudden, impulsive, high-flowrate aspiration of the gallstones. In the embodiment illustrated in FIG. 3, a “cross-slit” style valve is used as the first, distal valve 120;

alternatively, a duckbill valve, or any other style valve that would self-seal when exposed to the vacuum charge of the aspiration device 250 may be utilized equivalently.

Additionally, the aspiration device 250-250D (or syringe) can simply be inserted entirely through the first and second valves 120, 125 of the proximal hub 110 and a regular, non-bursting aspiration can be performed with the aspiration device 250-250D (or syringe) by slowly, or quickly, withdrawing the plunger 265 through the lumen 220 of the aspiration device 250-250D (or syringe). In another embodiments, the proximal hub 110, 110A may not have two or more valves (e.g., the first and second valves 120, 125), and instead may only have a single valve, or no valve at all. The proximal hub 110, 110A could have three or more valves as well to appropriately seal across a wide variety of instruments (or tools) and instrument sizes. In another embodiment, there may be no second, proximal valve 125 and all tools and aspirations are done directly through the central, single lumen 165, 555 of the tubular shaft 105, 505, or individual lumens of a multi-lumen tubular shaft 105, 505 that has accessed the targeted area. In another embodiment, a valve-less tubular shaft 105 can also be used as an accessing and upsizing dilator. These access dilators, or hub-less sheath shafts, can have an interior diameter to accommodate standard guidewires, or they can have an interior diameter that is sized to sequentially slide over the previous sized dilator. The medical personnel can continue to use larger and larger “dilators” or hub-less sheath shafts, or a balloon dilator or a balloon-tipped catheter, until the appropriate and desired sized dilation is reached. Access may be initiated, for example and without limitation, using a needle, such as an 18-21 gauge needle, followed by insertion of the dilator. The outer diameter of these shafts can range from small initial access sizes of 6-12 Fr (or smaller) and continue to increase up to 30 to 40 Fr (with 1 Fr=0.33 mm diameter or, equivalently, 3 Fr=1 mm diameter or 1 Fr=3×(diameter in mm)). In a system without a proximal hub 110, 110A, the aspiration device 250 (or syringe) or mechanisms described below, would attached directly to the tubular shaft 105 and lumen 165, in either a fixed manner, temporarily fixed (removable), or passively seated against the tubular shaft 105 to seal when the medical personnel places it for an aspiration.

The proximal hub 110, 110A and aspiration device 250-250D may also have two or more locking stages. A first locking stage locks the aspiration device 250-250D to the proximal hub 110, 110A to perform an impulse or burst vacuum (a “whoosh”), and a second, more advanced locking stage or position that advances the distal tip 275 of the aspiration device 250-250D (or syringe) through the first, distal valve 120 (e.g., a cross-slit valve) to release the charged vacuum. The medical personnel could also bypass the first stage and fully insert the aspiration device through both first and second valves 120, 125 to perform a manual aspiration, rather than an impulse or burst vacuum aspiration.

In a representative embodiment, an aspiration device 250-250D can be implemented as a locking syringe, such as a locking syringe available from Inari Medical, Inc. of Irvine, California, US, and disclosed in U.S. Patent Publication No. 2022/0039815A1 , which is incorporated by reference herein with the same full force and effect if set forth in its entirety herein. In a representative embodiment, and as illustrated in the various Figures, the aspiration device 250-250D includes a comparatively longer, larger bore tip 275. The inner diameter of the lengthened tip 275 would be the same, or larger, than the inner diameter of the access sheath 100, 100A, 200, 200A, 500, 500A, 600, 600A, 800, 900, to provide that there are no chokepoints or other constraints for gallstone removal. Additionally, the aspiration device 250-250D such as a syringe may include a proximal locking feature or valve 255 to perform an impulse or burst vacuum aspiration, and further may also incorporate a two-stage locking mechanism at the tip 275 to detachably connect the aspiration device 250-250D directly to the proximal hub 110, illustrated as distal valves (or locking mechanisms) 280, 285 in FIGS. 9 and 10, respectively. This distal locking mechanism or valve may also be a single stage if an impulse or burst vacuum aspiration is not desired, or the impulse or burst vacuum aspiration mechanism is built into aspiration device 250-250D itself. The locking mechanism may be implemented similarly to the Intri24 Sheath from Inari Medical, Inc. (U.S. Patent Application No. 63/307,766, also incorporated by reference herein with the same full force and effect if set forth in its entirety herein).

In another embodiment, the vacuum chamber may be confined to the aspiration device 250-250D itself. In a representative embodiment, this is accomplished by adding a flow control mechanism or valve 280, 285 to the tip 275 of the aspiration device 250, 250B, 250C, such as a stopcock, gate valve, rotary valve or ball valve, etc., illustrated in FIGS. 9 and 10. In this embodiment, the aspiration device 250B, 250C, while separate from the access sheath 100, 100A, 200, 200A, 500, 500A, 600, 600A, 800, 900, can be a closed system (valve closed) with the plunger 265 in the forward position. The plunger 265 can then be retracted to create a vacuum within the aspiration device 250B, 250C. The isolated vacuum charged aspiration device 250B, 250C can then be attached to the proximal hub 110. The flow control mechanism or valve 280, 285 can then be activated to create a quick transition from no-flow to high-flow aspiration (impulse or burst vacuum aspiration), or opened slowly to create a slow transient increase in aspiration flowrate, or partially opened to maintain a slow flowrate through the duration of the aspiration.

Separately, medical personnel can utilize the flow control mechanism or valve 280, 285 in the open position, and plunger 265 forward. Connecting the aspiration device 250-250D to the proximal hub 110 in this position, the user can retract the plunger 265 as quickly or slowly as they need or want to create a high flow or low flow aspiration.

Finally, the user such as medical personnel can also utilize the flow control mechanism or valve 280, 285 in the open position and plunger retracted (aspiration device 250B, 250C such as the illustrated syringe filled with a medium desired to be injected) and connect the aspiration device 250B, 250C to the proximal hub 110. The user can then infuse the contents of the aspiration device 250-250D to inflate the gallbladder, perform fluoroscopy, or use the infusion to manipulate the location of the gallstones. Similarly, using the one or more access ports 130, 130A, 135 of the access sheath 100, 100A, 200, 200A, 500, 500A, 600, 600A, 800, 900 can also be used to manipulate the position of the gallstones within the gallbladder.

Whether the syringe is injecting a medium or a pressurized saline bag is used to continuously inject a medium, this injection, or infusion, can move any gallstones. The direction that the infusion enters the gallbladder can be designed to push gallstones away from the access sheath 100, 100A, 200, 200A, 500, 500A, 600, 600A, 800, 900 and into a basket or cage 400 -400K (illustrated and described below), or from the far side of the gallbladder towards to the distal end 140 and interior lumen 165 of the access sheath 100, 100A, 200, 200A, 500, 500A, 600, 600A, 800, 900 to bring them closer for aspiration. The infusion can exit the access sheath 100, 100A, 200, 200A, 500, 500A, 600, 600A, 800, 900 straight out, at some desired angle, or even perpendicular to the access sheath 100, 100A, 200, 200A, 500, 500A, 600, 600A, 800, 900. The directionality of the infusion can be used and rotated to move stones around the gallbladder appropriately and as desired by the user.

Additionally, a separate instrument can be inserted through the interior lumen 165, or a selected lumen in a multi-lumen embodiment 300C, of the access sheath 100, 100A, 200, 200A, 500, 500A, 600, 600A, 800, 900 and positioned within the gallbladder. This separate instrument can also be used for infusion and manipulation of the position of the stones in relation to the gallbladder and the access sheath 100, 100A, 200, 200A, 500, 500A, 600, 600A, 800, 900. This separate instrument, like the access sheath 100, 100A, 200, 200A, 500, 500A, 600, 600A, 800, 900, can have its infusion port/stream direction designed to be straight, at any desired angle, perpendicular, but could also be pointed backwards, towards the access sheath 100, 100A, 200, 200A, 500, 500A, 600, 600A, 800, 900 to move stones towards the distal end 140 and interior lumen 165 of the access sheath 100, 100A, 200, 200A, 500, 500A, 600, 600A, 800, 900. The forward or angled injection can be used in the ducts of the gallbladder and biliary system (cystic duct, common bile duct, pancreatic duct, etc.) to dislodge gallstones from the ducts, in a direction that either moves them further along the duct pathway towards the duodenum, or small intestine, or to dislodge them from the ducts back into the gallbladder for removal by the access sheath 100, 100A, 200, 200A, 500, 500A, 600, 600A, 800, 900 and capture baskets (described below).

One important feature to note is that unlike current large bore aspiration devices, in a representative embodiment, there is no large bore side port through which material may be removed. Instead, in a representative embodiment, the aspiration device 250 -250D connects directly in line (linearly aligned) with the interior lumen 165 of the access sheath 100, 100A, 200, 200A, 500, 500A, 600, 600A, 800, 900. This is especially important in gallstone removal (as opposed to blood clot removal) due to the incompressible and non-uniform nature of gallstones. Depending on the composition of the gallstones (pigment stones versus cholesterol stones), they can be extremely hard and virtually rock-like in structure, and as a consequence, traversing through a side port which included an abrupt angle would not be ideal. Alternatively, in another representative embodiment, one or more access ports 130, 130A, 135 and/or other side ports can be provided and appropriately sized, e.g., with a comparatively large diameter bore, and utilized for aspiration, when given ample space for a large gallstone to make the transition from the interior lumen 165 of the access sheath 100, 100A, 200, 200A, 500, 500A, 600, 600A, 800, 900 through the side turn into the tubing of the access ports 130, 130A, 135. Additionally, an alternative embodiment may include a proximal aspiration source that is a flexible, self-expanding container in which gallstones could be collected and stored during removal. This aspiration source could be connected directly to the shaft 105, and is described in greater detail below with reference to FIG. 32.

As mentioned above, it should be noted that the proximal hub 110, 110A of the access sheath 100, 100A, 200 may not have two valves 120, 125, but it may have one valve, such as access sheath 200A, it may have 3 valves or more, or it may have no valves. If there are no valves, an aspiration device 250-250D can be permanently fixed, temporarily fixed, or passively “pressed against” the access sheath 100, 100A, 200, 200A, 500, 500A, 600, 600A, 800, 900 to create a seal. In any case, the “fixing” of the aspiration device 250 250D to the access sheath 100, 100A, 200, 200A, 500, 500A, 600, 600A, 800, 900 may be rigid, or flexible, or aspects of both depending on the component. Regardless of the proximal hub 110, 110A, valves, or no valves, the access sheath 100, 100A, 200, 200A, 500, 500A, 600, 600A, 800, 900 shaft may be straight, curved, tapered, twisting or stepped, such as the curved access sheath 100B illustrated in FIG. 32, for example and without limitation.

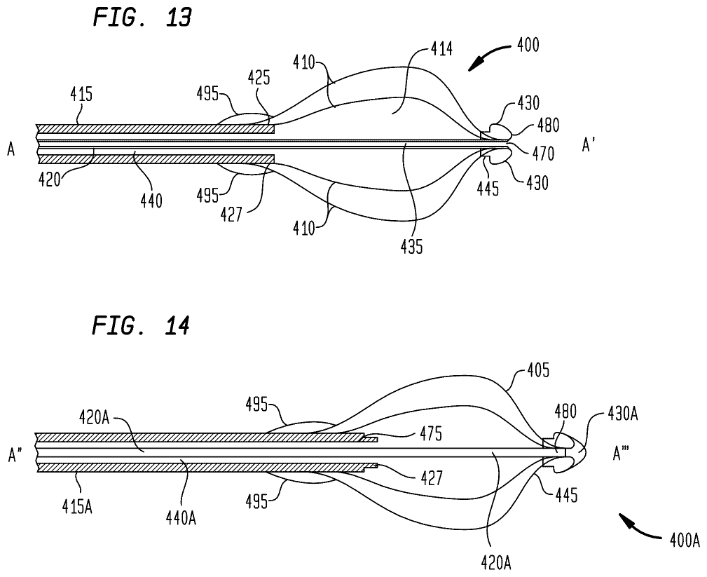

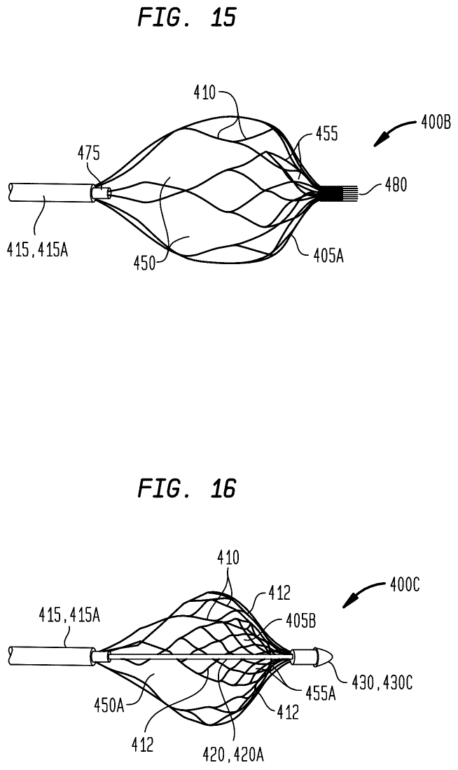

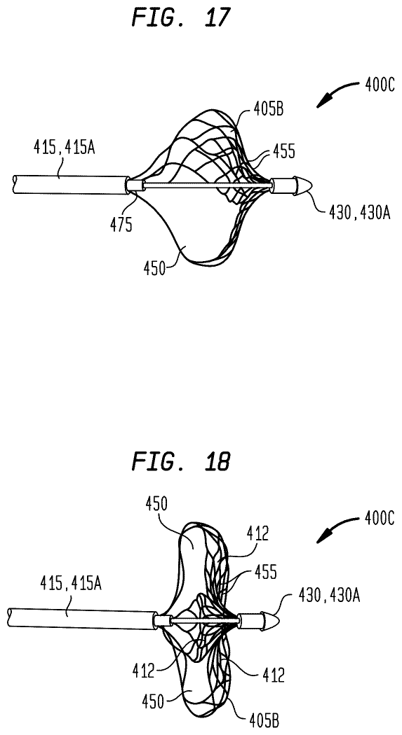

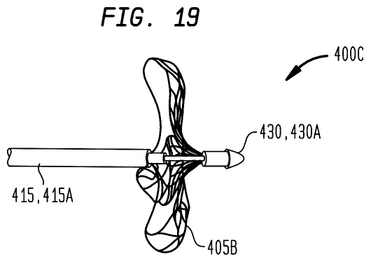

There are instances in which various instruments, in addition to the access sheath 100, 100A, 200, 200A, 500, 500A, 600, 600A, 800, 900 and aspiration device 250 250D, may be necessary or desirable for facilitating the removal of gallstones from the biliary tract or elsewhere within the subject or patient. For example and without limitation, when aspiration alone is not sufficiently effective, or the stones are too large to remove through the access sheath 100, 100A, 200, 200A, 500, 500A, 600, 600A, 800, 900, one or more additional instruments, such as a catheter apparatus 400-400H, may be necessary or desirable to capture and/or fracture the gallstone(s) and break them down into smaller, more readily removable pieces. A basket or cage 405-405K (e.g., comprised of nitinol) may be used to help collect and/or crush the gallstones to a smaller size for extraction, and may be utilized in addition to or in lieu of an aspiration device 250-250D. FIG. 11 is an isometric view illustrating representative first and second embodiments of a catheter apparatus 400, 400A having a basket or cage 405, illustrated in an expanded state. FIG. 12 is an isometric view illustrating the representative first and second embodiments of a catheter apparatus 400, 400A having a basket or cage 405, illustrated in the expanded state. FIG. 13 is a partial cross-sectional view illustrating the representative first embodiment of a catheter apparatus 400 having a basket or cage 405, illustrated in the expanded state. FIG. 14 is a partial cross-sectional view illustrating the representative second embodiment of a catheter apparatus 400A having a basket or cage 405, illustrated in the expanded state. FIG. 15 is an isometric view illustrating a representative third embodiment of a catheter apparatus 400B having a basket or cage 405A, and illustrated in an expanded state. FIG. 16 is an isometric view illustrating a representative fourth embodiment of a catheter apparatus 400C having a basket or cage 405B, illustrated in an expanded state. FIG. 17 is an isometric view illustrating the representative fourth embodiment of the catheter apparatus 400C having the basket or cage 405B, illustrated in an expanded state. FIG. 18 is an isometric view illustrating the representative fourth embodiment of the catheter apparatus 400C having the basket or cage 405B, illustrated in a contracted state. FIG. 19 is an isometric view illustrating the representative fourth embodiment of the catheter apparatus 400C having the basket or cage 405B, illustrated in the contracted state. Additional catheter apparatus 400-400H embodiments having additional basket or cage 405-405K embodiments are also described in greater detail below with reference to FIGS. 30A, 30B, and 52-57.

Referring to FIGS. 11-19, a representative embodiment of a catheter apparatus (or catheter) 400, 400A, 400B, 400C comprises an outer catheter tube (or outer catheter member) 415, 415A having an outer catheter lumen 440, 440A. An inner catheter manipulation shaft (or member) 420, 420A is arranged coaxially (or concentrically) within the outer catheter lumen 440, 440A and is moveable longitudinally (or longitudinally and rotatably) (e.g., push-pull with or without rotation) within the outer catheter lumen 440, 440A. The outer catheter tube (or outer catheter member) 415, 415A (and 415B) and inner catheter manipulation shaft (or member) 420, 420A (and 420B) may have any dimensions (lengths and diameters) suitable for insertion through an access sheath 100, 100A, 200, 200A, 500, 500A, 600, 600A, 800, 900 and into a subject gallbladder, or suitable for insertion coaxially within the shaft 835, 935 of a crushing or fracturing control apparatus 850, 950 which are then collectively inserted through an access sheath 100, 100A, 200, 200A, 500, 500A, 600, 600A, 800, 900 and into a subject gallbladder. In representative embodiments, not separately illustrated, the proximal end of the catheter 400-400H is not sealed or otherwise finished, such that the inner catheter manipulation shaft (or member) 420, 420A (and 420B) may be accessed and manipulated by the user relative to the outer catheter tube (or outer catheter member) 415, 415A (and 415B), or vice-versa, such as to expand or contract the basket or cage 405-405K.

As described in greater detail below with reference to FIGS. 30A and 30B, among others, the inner catheter manipulation shaft (or member) 420, 420A is a first shaft 420, 420A (and 420B) of the various catheter 400-400H embodiments, which may also include a crushing or fracturing instrument 700 having a (second) shaft 705 coupled to an angled or pointed crushing or fracturing tip 710 as an option, for example and without limitation. In representative catheter 400, 400A, 400C, 400D embodiments, a cage or basket 405, 405A, 405B is coupled both to the outer catheter tube 415, 415A and to the inner catheter manipulation shaft 420, 420A. The cage or basket 405, 405A, 405B comprises a plurality of struts 410, with the plurality of struts 410 arranged in a selected mesh crossing pattern, of a plurality of mesh crossing patterns, to form the corresponding cage or basket 405, 405A, 405B as a mesh, or variable or otherwise non-uniform mesh having a plurality of pores of differing sizes, as described in greater detail below. In the various mesh crossing patterns, the plurality of struts 410 may overlap each other or may merely abut or join each other without overlapping or crossing over one another. Any and all such mesh crossing patterns or arrangement of struts 410, in addition to those illustrated, are considered equivalent and within the scope of the disclosure. The plurality of struts 410 of the various cages or baskets 405, 405A, 405B are arranged to form a mesh having comparatively larger pores or openings 450 at the proximal region 460 of the cage or basket 405, 405A, 405B and have comparatively smaller pores or openings 455 at the distal region 465 of the cage or basket 405, 405A, 405B. In addition, in other representative embodiments, the plurality of struts 410 extend longitudinally without crossing. As described in greater detail below, the plurality of struts 410 of any of the cage or basket 405-405K embodiments may be comprised of any suitable material (e.g., nitinol or other metals or metallic alloys, carbon fiber, or any of various suitable polymers) in any suitable configuration to form a cage or basket 405-405K, including wire, braids, braided wire, etc.

More particularly in representative catheter 400, 400A, 400C, 400D embodiments, the plurality of struts 410 at a first end 425 of the cage or basket 405, 405A, 405B are coupled to the distal end 427 of the outer catheter tube (or member) 415, 415A, generally or typically coupled in between the outer catheter tube (or member) 415, 415A and a comparatively thin, generally cylindrical catheter tube collar (or cover) 495, so that any potentially abrasive or sharp ends of the struts 410 (at the first end 425) are covered by the catheter tube collar or cover 495. The plurality of struts 410 at a second end 445 of the cage or basket 405, 405A, 405B are coupled to a distal end 480 of the inner catheter manipulation shaft 420, 420A (which forms a catheter tip), generally or typically coupled in between the inner catheter manipulation shaft 420, 420A and a catheter tip cap 430, 430A, also so that any potentially abrasive or sharp ends of the struts 410 (at the second end 445) are covered by the catheter tip cap 430, 430A.

FIGS. 15 and 16 illustrate additional representative options for crossing patterns or arrangement of struts 410. As illustrated in FIG. 15, the cage or basket 405A has comparatively fewer struts 410, providing larger pores or openings 450 and smaller pores or openings 455, and without having a catheter tip cap 430, 430A at the distal end 480. For this catheter apparatus 400B embodiment, it should be noted that the inner catheter manipulation shaft (or member) 420 does not extend all the way to the distal end 480 or may be omitted altogether. As illustrated in FIG. 16, the cage or basket 405B has more struts 410 than cage or basket 405A and comparatively fewer struts 410 than cage or basket 405. The cage or basket 405B has three leaves 412 (in a 3-leaf cloverleaf pattern) of struts 410, providing three corresponding groups of smaller pores or openings 455A as illustrated, and providing three corresponding and comparatively large pores 450A. In an expanded state, the surface area of each of the large pores 450A is about the same or even larger than the surface area of each of the leaves 412, providing comparatively large pores 450A to capture stones, which can then be netted more tightly by the leaves 412. The cage or basket 405B also has a catheter tip cap 430, 430A at the distal end 480, and may use the hollow or solid inner catheter manipulation shaft (or member) 420 or 420A.

As illustrated, by moving the inner catheter manipulation shaft (or member) 420, 420A longitudinally within the outer catheter lumen 440, 440A, relative to the outer catheter tube or member 415, 415A, the cage or basket 405, 405A, 405B may be expanded or contracted. For example, as the distal end 480 is moved furthest away or furthest from the outer catheter tube or member 415, 415A, the shape of the cage or basket 405, 405A, 405B becomes more fusiform or elongated (spindle-shaped) and the diameter of the cage or basket 405, 405A, 405B is narrowed or contracted. As the distal end 480 is moved closer to the outer catheter tube or member 415, 415A, such as illustrated in FIGS. 11-16, the length of the cage or basket 405, 405A, 405B is shortened while the diameter of the cage or basket 405, 405A, 405B is expanded, resulting in the cage or basket 405, 405A, 405B having more of a pyriform (pear or fig) shape. As the catheter tip 480 is moved even closer to the outer catheter tube or member 415, 415A, such as illustrated in FIGS. 17-19, the length of the cage or basket 405, 405A, 405B is shortened even more while the diameter of the cage or basket 405, 405A, 405B is further expanded, resulting in the cage or basket 405, 405A, 405B having more of a disc shape.

The inner catheter manipulation shafts (or members) 420, 420A differ from each other insofar as the inner catheter manipulation shaft (or member) 420 is hollow and has an inner catheter shaft lumen 435, useful for flushing and moving gallstones, for example and without limitation, while the inner catheter manipulation shaft (or member) 420A is solid and does not have any such lumen. The outer catheter tubes 415, 415A may also differ from each other insofar as the outer catheter tube 415 may have a comparatively smaller diameter outer catheter lumen 440, abutting or only slightly spaced apart from the inner catheter manipulation shaft (or member) 420, while the outer catheter tube 415A may have a comparatively larger diameter outer catheter lumen 440A, spaced apart from the inner catheter manipulation shaft (or member) 420 and providing room for using the outer catheter lumen 440A for flushing and moving gallstones. Stated another way, using the inner catheter manipulation shaft (or member) 420, an infusion (such as a saline infusion) may be provided through the inner catheter shaft lumen 435 of the inner catheter manipulation shaft (or member) 420 and out through a corresponding opening of the distal catheter (catheter tip) (and catheter tip cap 430), referred to as a catheter distal flush port 470, and using the inner catheter manipulation shaft (or member) 420A, an infusion (such as a saline infusion) may be provided through the outer catheter lumen 440A of the outer catheter tube (or member) 415A and out through a corresponding outer catheter tube opening, referred to as a catheter proximal flush port 475 of the outer catheter tube (or member) 415A, illustrated in FIGS. 14 and 15. In this way, infusion or flushing may be provided at different locations of the catheter apparatus 400, 400A, 400B, 400C.

While not separately illustrated, it should be noted that these various embodiments may be provided in any combination, and all such combinations are within the scope of the disclosure. For example and without limitation, a catheter 400, 400A, 400B, 400C may be provided having both the inner catheter shaft lumen 435 of the inner catheter manipulation shaft (or member) 420 and the outer catheter lumen 440A of the outer catheter tube (or member) 415A, to provide selectable infusion or flushing at either or both locations, the catheter distal flush port 470 and/or the catheter proximal flush port 475. In addition to having these structural features and combinations of structural features, it should also be noted that the catheter apparatus 400, 400A, 400B, 400C also differs from other prior art catheters insofar as the cage or basket 405, 405A, 405B does not need to have a fully compressed state within the outer catheter tube (or member) 415, 415A, can be provided externally to the outer catheter tube (or member) 415, 415A, and does not need to be withdrawn into the outer catheter tube (or member) 415, 415A for capturing and removing gallstones. Alternatively, not separately illustrated, the cage or basket 405, 405A, 405B can be collapsed into a small delivery catheter and deployed inside the gallbladder via the access sheath 100, 100A, 200, 200A, 500, 500A, 600, 600A, 800, 900.

This catheter apparatus 400, 400A, 400B, 400C may be inserted through the access ports 130, 130A, 135, the plunger lumen 290 of the aspiration device 250, 250A, 250B, 250C, 250D, or just directly through the proximal hub 110, 110A of the access sheath 100, 100A, 200, 200A. For gallstone removal, it may be more desirable to insert through the plunger lumen 290 of the aspiration device 250, 250A, 250B, 250C, 250D or the hub lumen 195 of the proximal hub 110, 110A of the access sheath 100, 100A, 200, 200A, 500, 500A, 600, 600A, 800, 900 so that the catheter apparatus 400, 400A, 400B, 400C remains in the continuous, comparatively large diameter interior lumen 165 of the access sheath 100, 100A, 200, 200A, 500, 500A, 600, 600A, 800, 900. Once the cage or basket 405, 405A, 405B is expanded inside the gallbladder, the cage or basket 405, 405A, 405B may be manipulated (rotated, advanced, retracted, expanded, etc.) to capture gallstones within the plurality of struts 410 of the cage or basket 405, 405A, 405B. The user could then recapture the cage or basket 405, 405A, 405B in the delivery catheter and/or otherwise tighten the cage or basket 405, 405A, 405B around the stones for removal or crushing. Successive tightening of the cage or basket 405B is illustrated in FIGS. 16-20, as the cage or basket 405B is successively contracted from the expanded state. As the cage or basket 405, 405A, 405B is contracted, and as described in greater detail below, the captured gallstone may be fractured or crushed as well, reducing the sizes of the gallstone pieces to facilitate removal.

The cage or basket 405, 405A, 405B has variable pore sizes: comparatively more open on the proximal end and comparatively more closed on the distal end, as mentioned above. This variable pore size may help with capture and retainment of stones, using the larger pores or openings 450 to capture the gallstones(s) within the cage or basket 405, 405A, 405B, such as illustrated in FIG. 30A, followed by contracting the cage or basket 405, 405A, 405B around the captured gallstone, such that the gallstone is larger than and secured by the smaller pores or openings 455, such as illustrated in FIG. 30B. In other embodiments, the cage or basket 405, 405A, 405B may have uniform pore size smaller or larger than the illustrated examples. The catheter apparatus 400 also has a catheter distal flush port 470 at the catheter tip 430 which could help push gallstones towards the aspiration device 250, 250A, 250B, 250C, 250D rather than away with prior art flushing systems.

Alternatively, the flushing could be done at the proximal end of the cage or basket 405, 405A, 405B using the catheter proximal flush port 475 of the outer catheter tube (or member) 415A which would allow for more room for larger stones to be entrapped into the nitinol cage or basket 405, 405A, 405B.

In one embodiment the catheter distal flush port 470 at the catheter tip 430 has a flush lumen. This would allow the user to inject saline and push gallstones back towards the distal end 140 of the access sheath 100, 100A, 200, 200A for extraction. When flushing from the catheter proximal flush port 475 of the outer catheter tube (or member) 415, it is possible to push the gallstones further into the cage or basket 405, 405A, 405B, which may be useful for manipulating the locations of the gallstones. When flushing from the catheter distal flush port 470, it is possible to push the gallstones farther away, which also may be useful for manipulating the locations of the gallstones. Additionally, the expanded cage or basket 405, 405A, 405B could help prop the gallbladder open during use of the aspiration device (impulse or burst vacuum aspiration or manual). Due to the small volume of the gallbladder, it may collapse during aspiration, inhibiting the extraction of gallstones, so using the cage or basket 405, 405A, 405B to additionally help prevent that collapse may provide extra utility.

Another alternative version of the cage or basket 405, 405A, 405B is to have the flushing line act as a pull wire connecting the distal end of the catheter apparatus 400, 400A, 400B, 400C to a user controllable handle. This allows the user to customize the shape and diameter during the procedure for better control to gather the stones. This also allows for crushing of the stones by pulling the distal end of the cage or basket 405, 405A, 405B, compressing the stones between the struts.

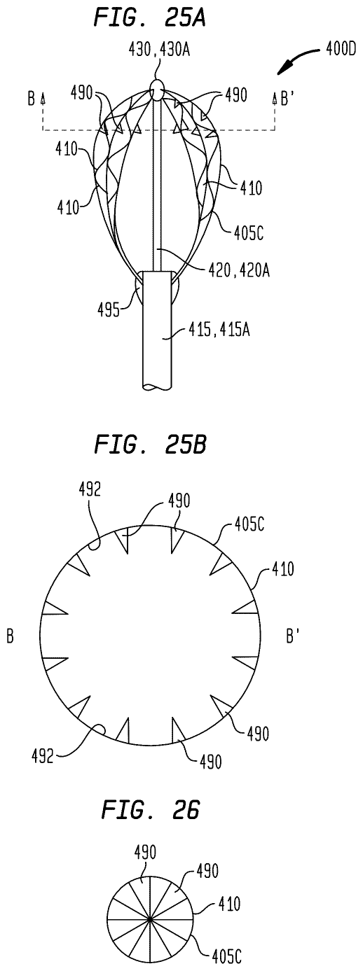

Additionally, as illustrated and described below with reference to FIGS. 25-28, one or more sharp points and/or hooks may be coupled within the cage or basket 405, 405A, 405B, either or both coupled to selected struts 410 or other components of the catheter 400, 400A, 400B, 400C, such as coupled within the cage or basket 405, 405A, 405B to the distal cap 430. This will assist breaking gallstones as the cage or basket 405, 405A, 405B is contracted, as all the inward compressive force will be concentrated to sharp points of contact.

FIG. 20 is a partial cross-sectional view (through the C-C′ plane of FIG. 1) illustrating a representative fourth embodiment of a system 300B having a first embodiment of the access sheath 100, additionally with a representative first or second embodiment of an aspiration device 250, 250A (or other aspiration device 250-250D), and with a representative catheter apparatus 400-400D embodiment. FIG. 20 illustrates the system 300B arrangement with a catheter 400-400D arranged coaxially (or in-line) with the access sheath 100 (or 200) and aspiration device 250-250D.

FIG. 21 is a partial cross-sectional view (through the C-C′ plane of FIG. 1) illustrating a representative fifth embodiment of a system 300C having a fourth embodiment of an access sheath 100A, with a representative catheter apparatus 400-400D embodiment. The access sheath 100A differs from an access sheath 100 insofar as the access sheath 100A has multiple interior lumens 165A, 165B, separated by one or more interior, longitudinal partitions or walls 168 to form the multiple, separate interior lumens 165A, 165B, to illustrate a multi-lumen embodiment described above. FIG. 21 illustrates the system 300C with the catheter 400-400D arranged through one or more access ports 130, 130A, 135, and may further include an aspiration device 250-250D (not separately illustrated). In addition, the system 300C includes a valve 295, such as a luer valve, within or coupled to the access port 130 and surrounding and sealing the catheter 400-400D. In this embodiment, the access sheath 100A has additional lumens 165A, 165B, that may be utilized for flushing of the gallbladder or introduction of various tools or drugs necessary to complete the procedure. This embodiment and other embodiments could also utilize a beveled distal end 140 on the access sheath 100A, which may help with procedure flow, including, but not limited to flushing, insertion, and angled aspiration.

FIG. 22 is a partial cross-sectional view (through the C-C′ plane of FIG. 1) illustrating a representative sixth embodiment of a system 300D having a first embodiment of the access sheath 100 illustrated in FIG. 1, additionally with a representative catheter apparatus 400-400D embodiment, and serves to illustrate a catheter 400-400D arranged coaxially (or in-line) with the access sheath 100 or 200, through the proximal hub 110, 110A and the first and second valves 120, 125, and optionally may further include an aspiration device 250-250D (not separately illustrated).

It should be noted that while FIGS. 20-22 are illustrated using a representative catheter apparatus 400-400D embodiment having a representative cage or basket 405-405D, those having skill in the field will recognize that any of the catheters 400 -400H having any of the cages or baskets 405-405K may be utilized equivalently, and any and all such variations are within the scope of the disclosure.

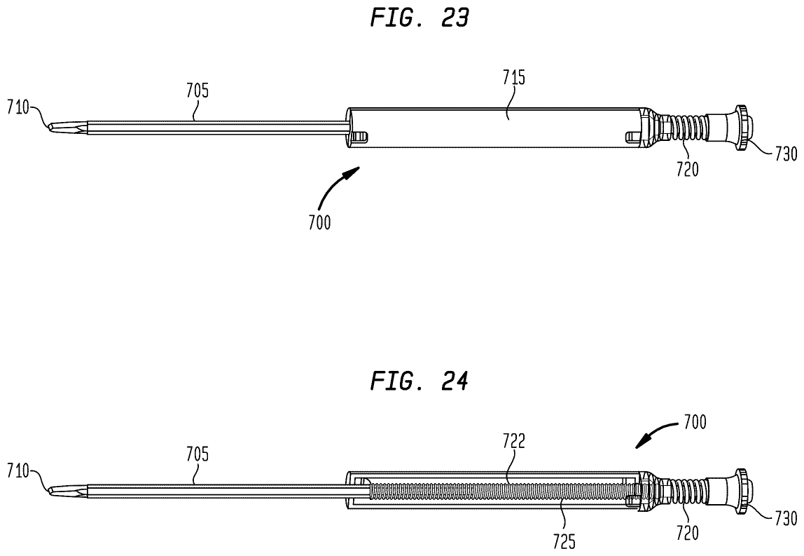

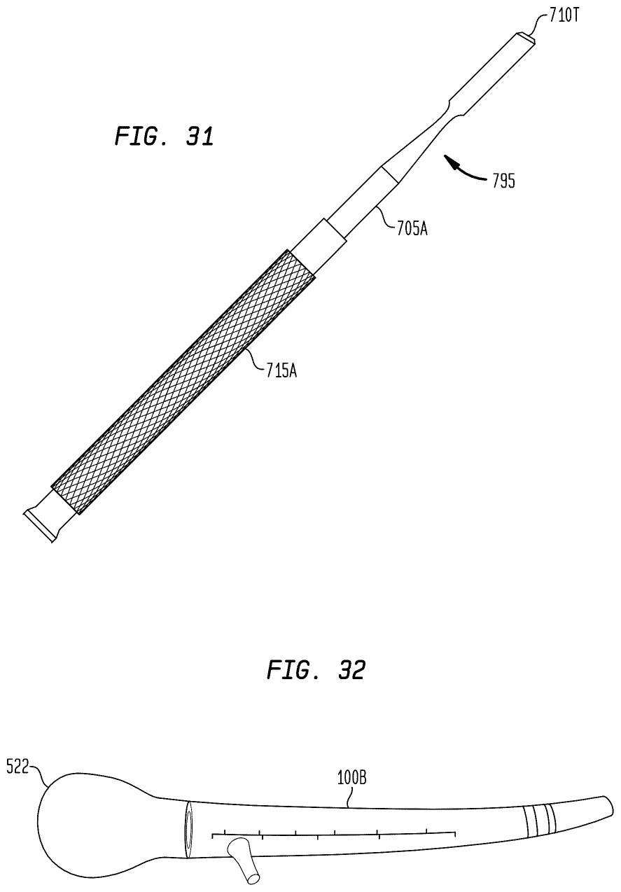

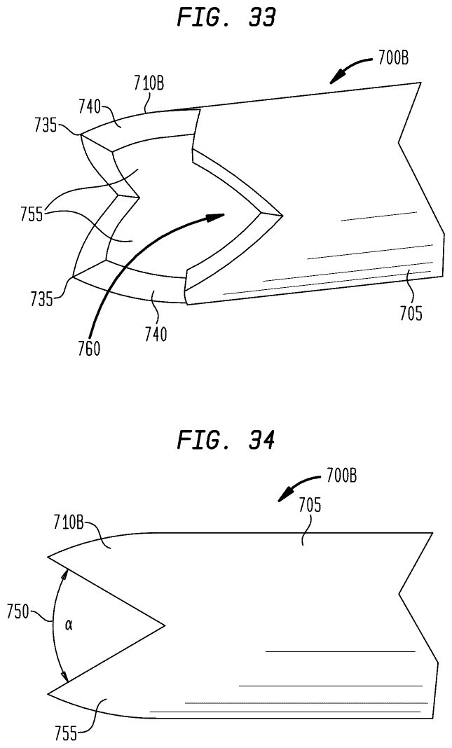

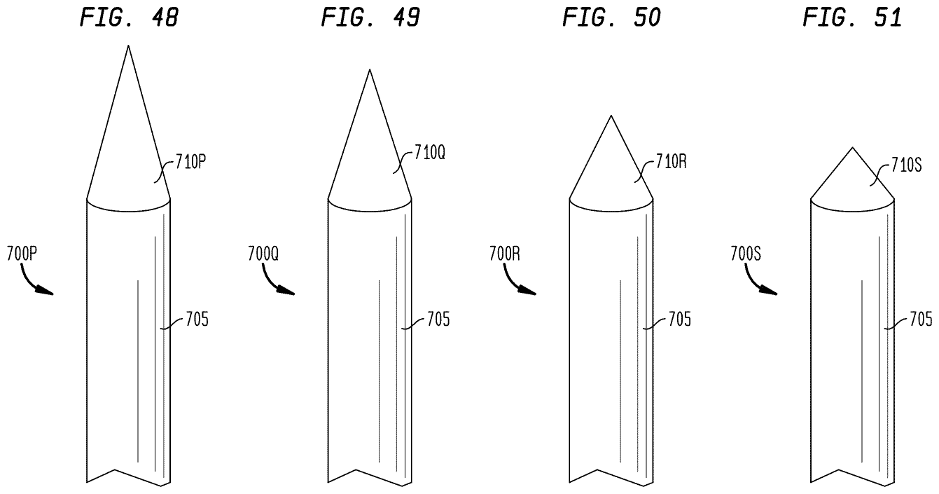

FIG. 23 is an isometric view illustrating a first embodiment of a representative crushing or fracturing instrument 700, for use as part of any of the various systems 300-300J. FIG. 24 is a cut-away view illustrating the representative first crushing or fracturing instrument 700, illustrating a second crushing or fracturing spring 722. A representative second embodiment of a crushing or fracturing instrument 795 is illustrated in FIG. 31, and additional embodiments of crushing or fracturing instruments are illustrated and described below with reference to FIGS. 33-51.

Referring to FIG. 23, the first embodiment of a crushing or fracturing instrument 700 comprises a flexible, hollow, partially hollow, or solid generally cylindrical crushing or fracturing shaft (or tubing) 705 having, integrally formed with or coupled to a first angled or pointed crushing or fracturing tip 710, with the crushing or fracturing shaft 705 arranged in a first crushing or fracturing instrument lumen 725 of a first crushing or fracturing handle 715, and further comprises a first crushing or fracturing spring 720, a second crushing or fracturing spring 722, and a first crushing or fracturing retraction actuator 730. The crushing or fracturing shaft 705 and crushing or fracturing tip 710 may be fabricated with any diameters and lengths suitable for use with any of the catheter 400-400H and access sheath 100, 100A, 200, 200A, 500, 500A, 600, 600A, 800, 900 embodiments. When mechanical crushing techniques with only a basket or cage 405-405K is not sufficient, then another mechanical tool such as a crushing or fracturing instrument 700, 795 can be introduced through the lumen 165 of the access sheath 100, 100A, 200, 200A or central lumen 555 of an access sheath 500, 500A, 600, 600A, 800, 900 described below. Other embodiments of crushing or fracturing instruments 700A-700S designed for use with other catheter 400 400H embodiments having inner catheter lumens 435 are described in greater detail below with reference to FIGS. 30A, 30B, and 33-51. The introduction of the crushing or fracturing instrument 700, 795 having the first angled or pointed crushing or fracturing tip 710 or second, sharp-edged or beveled crushing or fracturing tip 710T, respectively, can be used to crack, fracture, or crush the gallstone(s), and may have any suitable or appropriate crushing or fracturing tip 710, 710T geometry, in addition to the illustrated examples and to the crushing or fracturing tips illustrated in and described below with reference to FIGS. 33-51. The first angled or pointed crushing or fracturing tip 710 or second, sharp-edged or beveled crushing or fracturing tip 710T would be mounted on or be part of the flexible, hollow, partially hollow, or solid generally cylindrical shaft (or tubing) 705, 705A for direct transfer of force from the proximal end through the first angled or pointed crushing or fracturing tip 710 or second, sharp-edged or beveled crushing or fracturing tip 710T to the gallstone.

Additionally, energy can be stored by spring loading the first crushing or fracturing instrument 700, as illustrated in FIGS. 23-24, by using the first crushing or fracturing retraction actuator 730 to retract the crushing or fracturing shaft 705 of the first crushing or fracturing instrument 700 within the first crushing or fracturing instrument lumen 725 (relative to the first crushing or fracturing handle 715) and compressing the second crushing or fracturing spring 722. For example, as illustrated in FIG. 24, when the first crushing or fracturing instrument 700 is inserted through an access sheath 100, 100A, 200, 200A, 500, 500A, 600, 600A, 800, 900 and positioned at a selected or desired location within the subject gallbladder, with the second crushing or fracturing spring 722 in a compressed state, the second crushing or fracturing spring 722 may be released (such as by releasing the first crushing or fracturing retraction actuator 730), and the first angled or pointed crushing or fracturing tip 710 of the first crushing or fracturing shaft 705 is driven by the movement of the first crushing or fracturing spring 720 to impact a gallstone captured in a catheter 400 400H, to fracture the gallstone. This can be performed numerous times to fracture the gallstone into fragments sufficiently small to be removed, i.e., with repeated efforts of capturing and then breaking the stone to a diameter less than the inner diameter of a lumen 165, 165A, or 165B of the access sheath 100, 100A, 200, 200A or central lumen 555 of an access sheath 500, 500A, 600, 600A, 800, 900, all stones can be removed through the access sheath 100, 100A, 200, 200A, 500, 500A, 600, 600A, 800, 900 with the aid of the crushing or fracturing instrument 700, 795 (or other crushing instruments described in greater detail below) and collecting device such as the cage or basket 405-405K or other collecting devices also described in greater detail below. The crushing or fracturing instruments 700A 700S function similarly to impact and fracture gallstones and gallstone fragments. The first and second crushing or fracturing instruments 700, 795 and other crushing or fracturing instruments 700A-700S may be comprised of any suitable material, such as titanium, stainless steel, a polymer, for example and without limitation.

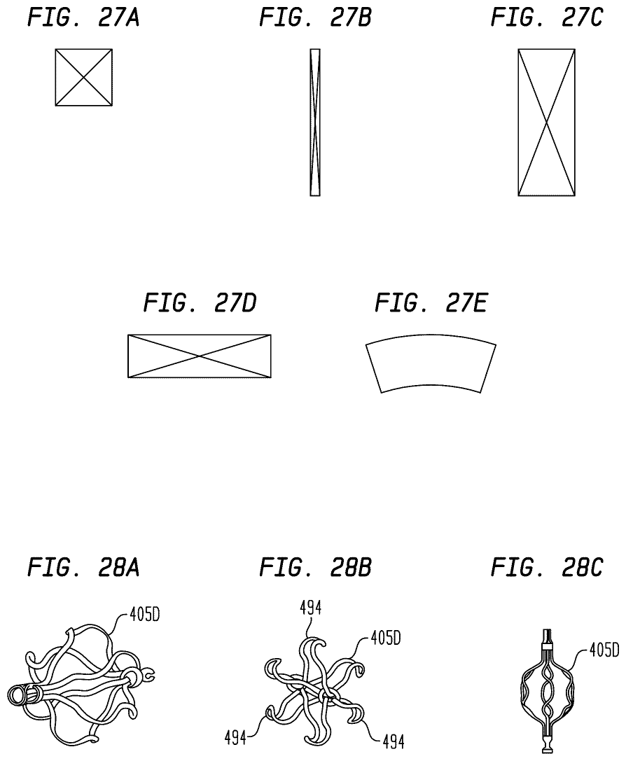

FIG. 25A is an isometric view illustrating a representative fifth embodiment of a catheter apparatus 400D having a representative fourth embodiment of a basket or cage 405C having representative sharp teeth, points, or wedges 490 coupled to or integrally formed with one or more interior surfaces 492 of one or more struts 410, illustrated in an expanded state. FIG. 25B is a cross-sectional view, through the B-B′ plane of the basket or cage 405C illustrated in FIG. 25A, illustrating the cage or basket 405C having representative sharp teeth, points, or wedges 490 coupled to or integrally formed with one or more interior surfaces 492 of one or more struts 410, illustrated in the expanded state. FIG. 26 is a cross-sectional view illustrating the basket or cage 405C having sharp teeth, points, or wedges 490 coupled to or integrally formed with one or more interior surfaces 492 of one or more struts 410, illustrated in a contracted state. FIGS. 27A, 27B, 27C, 27D, and 27E are cross-sectional views illustrating representative configurations of sharp teeth, points, or wedges 490 coupled to or integrally formed with one or more interior surfaces 492 of struts 410 of any of the representative baskets or cages 405, 405A, 405B, 405C of any of the various representative catheter 400, 400A, 400B, 400C, 400D embodiments, illustrated in contracted states. As mentioned above, one or more sharp teeth, points, or wedges 490 may be coupled to or integrally formed with the interior surfaces 492 of one or more struts 410 of any of the various baskets or cages 405, 405A, 405B to form a basket or cage 405C, either or both coupled to or formed with one or more selected struts 410 or other components of a basket or cage 405, 405A, 405B of the catheter 400, 400A, 400B, 400C to form a basket or cage 405C, and/or such as coupled to or integrally formed with the distal cap 430 and/or one or more struts 410 of a basket or cage 405, 405A, 405B to form a basket or cage 405C. This will assist breaking gallstones as the basket or cage 405C is contracted, as all the inward compressive force will be concentrated to sharp points of contact. As mentioned above, the basket or cage 405C may be utilized in place of or otherwise substituted for any of the representative baskets or cages 405, 405A, 405B of any of the catheter 400, 400A, 400B, 400C embodiments, and all such combinations and variations are considered equivalent and within the scope of the disclosure. In addition, any of the various baskets or cages 405 405K may also further comprise one or more sharp teeth, points, or wedges 490 coupled to or integrally formed with one or more interior surfaces 492 of one or more struts 410, of any of the various catheter 400-400H embodiments.

In order to reduce the overall force needed to crack a gallstone, the force can be concentrated on fewer individual sharp teeth, points, or wedges 490. To achieve this with a basket-type capture device followed by compression, the interior (or inner) surface 492 of the struts 410 may further comprise sharp teeth, points, or wedges 490 to form a basket or cage 405C. These sharp teeth, points, or wedges 490 concentrate the pressure/cracking force by applying the force to a smaller, and potentially fewer, point(s) of contact. One way to manufacture this basket or cage 405C is similar to the manufacture of the coring elements for ClotTriever and FT2 available from Inari Medical, Inc. Instead of cutting from a tubing (hollow inside), however, a large mandrel/wire may be utilized. This would leave struts of a basket or cage 405C that have a cross-sectional profile similar to a slice of pizza, or a dagger, as illustrated in FIGS. 25A, 25B, and 26. FIG. 26 illustrates an “as-cut-profile” of the basket or cage 405C. These cut pieces of the wire forming the sharp teeth, points, or wedges 490 can be heat set into an expanded shape of any of the various baskets or cages 405, 405A, 405B to form a basket or cage 405C, and would have a series of dagger struts circumferentially (not necessarily uniform, and not necessarily non-uniform) as shown in FIGS. 25A and 25B of the “as-expanded sharp” basket or cage 405C. The struts 410 having the sharp teeth, points, or wedges 490 could be as few as two and as many 24, for example and without limitation, and as previously mentioned, the basket or cage 405C can be designed with a reduced number of struts 410 on the proximal side and increased number struts 410 on the distal side, to create a less dense/open porous side, and a more dense/small porous side. A wire having a triangular cross-section may also be utilized directly to form some or all of the plurality of struts 410 of any of the various baskets or cages 405, 405A, 405B to form a basket or cage 405C, or to form the sharp teeth, points, or wedges 490, including grinding away portions of such triangular wires to leave sharp teeth, points, or wedges 490 remaining. As additional alternatives, the entire basket or cage 405C structure could have the same number of struts 410, or could have fewer struts on the distal side and more on the proximal side. Additionally, the proximal and distal ends of the basket or cage 405C could have a dense structure or less dense strut structure in comparison to the middle, and vice versa. Although, it is generally beneficial to have fewer struts as this reduces the overall force required to be provided by the user or the user control system in order to achieve the same pressure/cracking force applied per strut 410 to the gallstone. Separately, fewer struts 410 generally provides less overall strength of the system to retract the stones through the sheath, and also provides less structure to capture the stone in a basket-like device.

In another embodiment, the strut profile may not be triangular, or dagger/pizza shaped, and it may not have a sharp point on the interior contact surface of the strut 410. The strut profile can also simply be generally square, rectangular, round, or designed with an arc-like shape, such as illustrated in FIGS. 27A-27E.

FIGS. 28A, 28B, and 28C are isometric views illustrating a fifth embodiment of a basket or cage 405D having grasping hooks 494 for use with any of the various representative catheter 400-400D embodiments, illustrated in an expanded state. The basket or cage 405D can collect gallstones in a rotating manner, scooping up the gallstones, and may also be contracted or collapsed to crush the gallstones, and serves to illustrate the plurality of available shapes and functionality for any of the various baskets or cages disclosed herein.

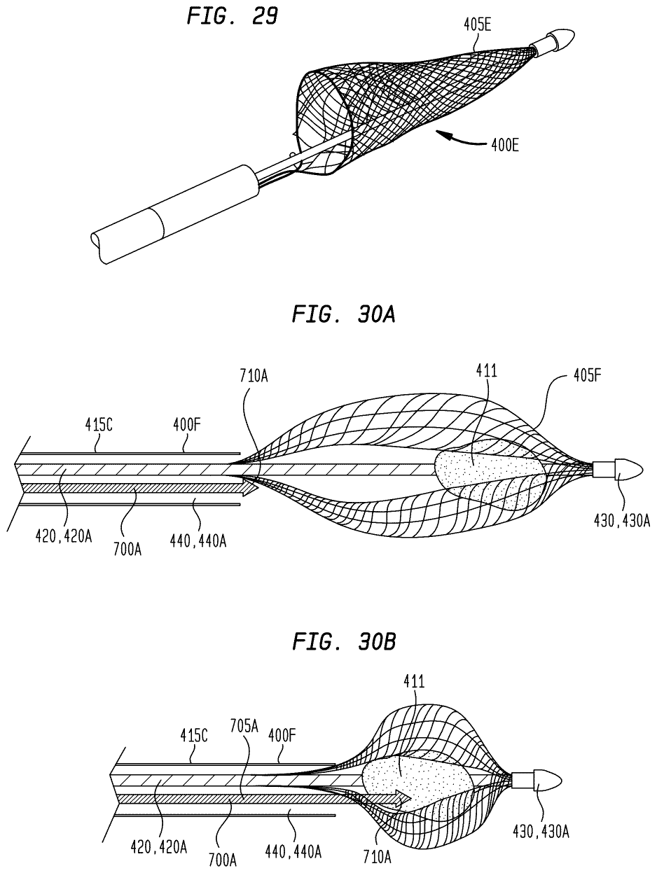

FIG. 29 is an isometric view illustrating a representative sixth embodiment of a catheter 400E apparatus having a sixth embodiment of a basket or cage 405E, illustrated in the expanded state, with the basket or cage 405E having a proximal hoop design (wire or laser cut) with an attached, braided collection basket.

In any of the various embodiments, the basket or cage 405-405K or other capturing device may be designed and manufactured from a material other than nitinol, and it may or may not be laser cut, or wire electrical discharge machined (EDM), as a single body basket. The basket or cage 405-405K or other capturing device can also be designed and built from a braided structure, individual wires running longitudinally, or a large scooping device that is not symmetrical (e.g., similar to an ice cream scooper). Outside of the cross-sectional profile of the struts 410, the overall basket or cage 405-405K or other capturing device can be any number of shapes, e.g., cylindrical, spherical, square, rectangular, trapezoidal, diamond, hexagonal, octagonal, etc. The basket or cage 405-405K or other capturing device can be symmetrical front to back, a circular pattern, or it could be non-symmetrical with one large side opening and one closed side. The structure can also be generally unique in each distinct area with little to no uniformity.

In some embodiments, the basket or cage 405-405K or other capturing device is connected to a stiff shaft or rod that extends through, and is moveable longitudinally or both longitudinally and rotatably within the outer catheter lumen 440, 440A, and can be controlled by the user. In other embodiments, the basket or cage 405-405K or other capturing device is attached to inner catheter manipulation shaft (or member) 420, 420A within a flexible outer catheter tube (or member) 415, 415A, with the inner catheter manipulation shaft (or member) 420, 420A being moveable longitudinally or both longitudinally and rotatably within the outer catheter lumen 440, 440A. In both embodiments the inner catheter manipulation shaft or member 420, 420A can be a wire, a tube, a concentric telescoping system, and these inner catheter manipulation shafts or members can be polymeric, with metallic support structure, polymeric without metallic support structure, and metallic only. The proximal portion of the basket or cage 405-405F or other capturing device can simply terminate in the ends of the wire(s) or tube(s) or it can terminate in a handle that the user can manipulate to guide, size, open, close, extend, retract or otherwise manipulate the basket or cage 405-405K or other capturing device. The basket or cage 405 -405K or other capturing device may come with the capture element in the normally collapsed position for the user to “expand” it once inside the gallbladder. Alternatively, the basket or cage 405-405K or other capturing device may be in the open/expanded position, and the user may collapse it prior to insertion through the access sheath 100, 100A, 200, 200A, 500, 500A, 600, 600A, 800, 900 and into the gallbladder. Alternatively, the basket or cage 405-405K or other capturing device may come packaged within a delivery system and the user should deploy the element from the delivery system to capture gallstones. In this embodiment, the device may come either normally collapsed or normally open once deployed from the delivery system. The user can then manipulate the basket or cage 405-405K or other capturing device appropriately to engulf the stones.

In some embodiments, the distal end and proximal end of the basket or cage 405-405K or other capturing device may be connected with an inner catheter manipulation shaft or member 420, 420A running through an interior 414 of the basket or cage 405-405K or other capturing device to the distal end 480 (for user control to open/expand and close/contract the basket or cage 405-405K or other capturing device), such as illustrated in FIGS. 11-14, 16-19 and 30A, 30B . In some embodiments, this inner catheter manipulation shaft (or member) 420, 420A may also have an inner catheter shaft lumen 435. In some embodiments, such as illustrated in FIG. 15, the interior of the basket or cage 405A does not include an inner catheter manipulation shaft (or member) 420, 420A, and instead has a large open void in the middle of the basket or cage 405A where gallstone(s) can be captured.

FIGS. 30A and 30B are isometric and partial cross-sectional views illustrating the capture and cracking or crushing of a gallstone using a representative seventh embodiment of a catheter 400F apparatus having a basket or cage 405F or any of the other various catheters 400-400H or baskets or cages 405-405K, with the basket or cage 405F capturing the gallstone 411 (FIG. 30A) and then retracting around the gallstone 411 (FIG. 30B) (with the basket or cage 405F illustrated using an isometric view and the balance of the catheter 400F simplified and illustrated in (partial) cross-section to illustrate the capturing and crushing/fracturing concepts). The catheter 400F comprises an outer catheter tube (or outer catheter member) 415C having an outer catheter lumen 440, 440A, an inner catheter manipulation shaft (or member) 420, 420A moveable longitudinally and rotatably within the outer catheter lumen 440, 440A, and a basket or cage 405F coupled both proximally and distally to the inner catheter manipulation shaft (or member) 420, 420A as illustrated. In various embodiments, the basket or cage 405-405K or other capturing device may not only rely on the struts or braid to break up the captured gallstones. Additional methods of fracturing stones can be included with or within the catheter 400-400H. One of these methods includes using a crushing or fracturing instrument 700-700S moveable longitudinally or both longitudinally and rotatably within the inner catheter lumen 435 (of an inner catheter manipulation shaft 420B) or within the outer catheter lumen 440, 440A (as illustrated in FIGS. 30A and 30B), to provide a “jack hammer” or “spear-like” (e.g., sharp or pointed) crushing or fracturing instrument 700-700S that can be used to forcefully engage the gallstone and crack it. This can be used in conjunction with the any of the types and shapes of struts 410 and basket or cage 405-405K or other capturing device disclosed herein. The crushing or fracturing tip 710-710T can be rounded, pointed, spear headed, diamond, chamfered, chiseled, bull nosed, bullet head, hollow, drill bit, hole saw, dimpled, screw/helical shaped and also fin-like, etc., and many are illustrated and described below, in addition to the spear-like crushing or fracturing tip 710A illustrated in FIGS. 30A, 30B. The action of this crushing or fracturing instrument 700-700S is to forcefully engage with and fracture the gallstone into smaller pieces, and this action may be accomplished using one or more of the following crushing or fracturing instrument 700-700S driving mechanisms: linear actuator, compression spring driven, extension spring drive, torsion spring driven, spring driven, constant force spring driven, user driven and controlled, ratcheting, rotating, and it can also be a helical, rotating lead screw like drive pattern. The crushing or fracturing instrument 700-700S can be fast-moving and have rapid impact(s), such as a single rapid impact, like a hammer, or a repetitive impact like a jack hammer. Once the crushing or fracturing tip 710-710T is embedded into the gallstone it can be withdrawn to cause additional fracturing within the gallstone. The crushing or fracturing tip 710-710T can also have a slow-moving engagement with the gallstone in a linear manner or in a rotational manner, similar to a drill bit. Again, this can be a forward and/or backward engagement, or both. In another embodiment, the crushing or fracturing instrument 700-700S can also be a slow or fast moving, forward or backward motion, that is helical or a screw like drive. This sort of drive motion would move rotationally and linearly. The rotational motion can then also drive the forward motion (coupled) like a gear or helical track, or the rotational motion and forward motion can be decoupled. The rotational motion can apply a drill-like engagement with the gallstone and the linear motion can be forcefully added by the user, or a spring-like or actual spring designed system. The screw-like tip or hole-saw tip can be embedded into the gallstone and then retracted forcefully to fracture the stone. Several devices and methods of accomplishing this are illustrated and described in greater detail below with reference to FIGS. 91-97.

Once a gallstone has been captured by the basket or cage 405-405K or other capturing device, the inner catheter manipulation shaft (or member) 420, 420A, 420B or pull wire(s) of the capturing element, can be locked in place to maintain the captured gallstone. In an additional embodiment, the inner catheter manipulation shaft (or member) 420, 420A, 420B or pull wire(s) can be retracted to apply tension and compression of the struts 410 of the basket or cage 405-405K or other capturing device onto the gallstone. The inner catheter manipulation shaft (or member) 420, 420A, 420B or pull wire(s) can continually be retracted into the delivery system (if applicable) to continue to apply an increasing compression force. The entire basket or cage 405-405K or other capturing device can also independently or simultaneously be pulled into the access sheath 100, 100A, 200, 200A, 500, 500A, 600, 600A, 800, 900 to apply another compression or fracturing force. This can be done independently of or in conjunction with the action of the inner catheter manipulation shaft (or member) 420, 420A, 420B or pull wire(s). Alternatively, both inner catheter manipulation shaft (or member) 420, 420A, 420B or pull wire(s) providing contraction and/or the retraction of the basket or cage 405-405K or other capturing device into the access sheath 100, 100A, 200, 200A, 500, 500A, 600, 600A, 800, 900 can be performed, and the crushing or fracturing instrument 700-700S can be actuated to create a third method of forcefully fracturing the targeted gallstone or obstruction. In other embodiments, the inner catheter manipulation shaft (or member) 420, 420A, 420B or pull wire(s) may be connected to the proximal end of the basket or cage 405-405K or other capturing device, or they may be individually connected to the strut(s) 410 of the basket or cage 405-405K or other capturing device. In other embodiments the inner catheter manipulation shaft (or member) 420, 420A, 420B or pull wire(s) on the proximal end may be the same unibody structure with the basket or cage 405 405K or other capturing device of the catheter 400-400H, where the basket or cage 405 405K or other capturing device of the catheter 400-400H is cut and expanded from the wire or lumen that extends to the proximal end.

In some embodiments the proximal end of the inner catheter manipulation shaft (or member) 420, 420A, 420B or pull wire(s) or strut(s) 410 of the basket or cage 405 405K or other capturing device may be connected to a retraction mechanism in addition to, or separately from the application of force from a crushing or fracturing instrument 700-700S mentioned above. This retraction mechanism can pull on the inner catheter manipulation shaft (or member) 420, 420A, 420B or pull wire(s) or strut(s) 410, which applies compression on the gallstone, by using a linear actuation, spring driven actuation, linear rachet, rotating ratchet, lead screw, motor driven, or manual, and any other forms of applying a pulling force to a single wire or set of wires. As a result, the basket or cage 405-405K itself can compress, fracture, and/or crush one or more gallstones. In addition, the basket or cage 405 405K can be retracted into the outer catheter lumen 440, 440A, also providing contraction to compress, fracture, and/or crush one or more gallstones.

In other embodiments, the inner catheter manipulation shaft (or member) 420, 420A or pull wire(s) or strut(s) 410, may be constrained in a guide on the distal end of the catheter 400-400E, or the distal end of the access sheath 100, 100A, 200, 200A, 500, 500A, 600, 600A, 800, 900, to prevent the struts 410 from sliding to one side of the gallstone while applying compression to the gallstone after capturing it. Containing the struts 410 can be done with a castellated/crown shaped distal tip, or a multi-lumen disk like structure that is fixed to the distal end of the catheter 400-400H.

In another embodiment, the distal portion of the basket or cage 405-405K or other capturing device can be somewhat open to release fractured gallstones. This singular opening, or multi-openings/pores can be sized to be the same size, or smaller, in comparison to the aspiration lumen 165 of the access sheath 100, 100A, 200, 200A, 500, 500A, 600, 600A, 800, 900.

FIG. 32 is a side, elevational view illustrating the representative fifth embodiment of an access sheath 100B with proximal fixed aspiration from a flexible, self-expanding member 522. In some embodiments the proximal hub 110, 110A can utilize a proximal hemostasis valve from the FlowTriever and ClotTriever product lines available from Inari Medical, Inc. In other embodiments, the proximal hub 110, 110A may utilize a series of valves 120, 125. In other embodiments, the proximal hub 110, 110A can also provide the source or suction/vacuum for the aspiration, such as through the access ports 130, 130A, 135 or in-line port 152. A vacuum source, such as the bulbous flexible, self-expanding member 522, can be coupled to the proximal hub 110, 110A, and the flexible, self-expanding member 522 can be compressed by the user to reduce the inner volume of the flexible, self-expanding member 522 and then released by the user to allow the flexible, self-expanding member 522 to expand and create a vacuum having an aspiration/withdrawing flowrate. This action can pull the gallstones out of a gallbladder. In addition, the flexible, self-expanding member 522 and/or proximal hub 110, 110A can contain a series of valves, such as the first and second valves 120, 125. One valve can be distal to the flexible, self-expanding member 522, located within or near the shaft 105 of the access sheath 100, 100A, 200, 200A, 500, 500A, 600, 600A, 800, 900, and can limit the flow of fluid to be directed outwards, and limit the inward flow of fluid, unless a tool is passed through the valve to bypass it. Additionally, a second valve can be included on the proximal side of the flexible, self-expanding member 522 that allows fluid to further travel outward but limit inward flow. The series of these two valves will keep fluid and stones moving outward and not be capable of flowing inward toward the gallbladder.

As described above with reference to FIGS. 30, 30B, and as described in greater detail below, various embodiments of catheters 400-400H, such as catheters 400F, 400G, 400H, comprise an outer catheter tube (or outer catheter member) 415B having an outer catheter lumen 440B. An inner catheter manipulation shaft (or member) 420B is arranged coaxially (concentrically) within the outer catheter lumen 440B and is moveable longitudinally (or longitudinally and rotatably) (e.g., push-pull with or without rotation) within the outer catheter lumen 440B. The inner catheter manipulation shaft (or member) 420B has an inner catheter shaft lumen 435, as an option, for example and without limitation, configured and sized for insertion of a crushing or fracturing instrument 700-700S. For example, when a gallstone has been captured and secured within a cage or basket 405-405K (such as by retracting the struts 410 around the gallstone and into the outer catheter lumen 440B), a crushing or fracturing instrument 700-700S may be inserted through the inner catheter shaft lumen 435 and utilized to fracture the gallstone. As described in greater detail below, when the catheter 400F, 400G, 400H has been inserted through an access sheath 100, 100A, 200, 200A, 500, 500A, 600, 600A, 800, 900 and positioned at a selected or desired location within the subject gallbladder, following capture of a gallstone, the basket or cage 405-405K may be retracted around the gallstone, the crushing or fracturing instrument 700 700S may be inserted through the inner catheter shaft lumen 435 and positioned near or abutting the captured gallstone, and the crushing or fracturing instrument 700-700S may be actuated, resulting in the angled or pointed crushing or fracturing tip 710-710S impacting the gallstone captured in the catheter 400F, 400G, 400H, to fracture the gallstone. This is particularly useful for gallstones or gallstone fragments which are larger than the inner lumen 165 of the access sheath 100, 100A, 200, 200A or central lumen 555 of an access sheath 500, 500A, 600, 600A, 800, 900, e.g., greater than about 8 mm. The fractured gallstone or gallstone fragment may then be removed from the subject gallbladder using the catheter 400F, 400G, 400H or by aspiration, for example and without limitation.

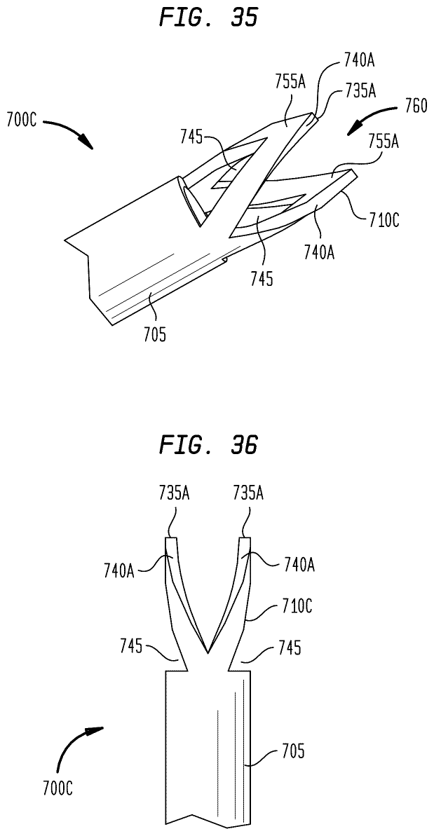

A wide variety of crushing or fracturing instruments 700B-700S having a wide variety of crushing or fracturing tip 710B-710 S geometries are illustrated in FIGS. 33-51. FIG. 33 is a partial isometric view illustrating a representative third embodiment of a crushing or fracturing instrument 700B. FIG. 34 is a partial, side, elevational view illustrating the representative third embodiment of the crushing or fracturing instrument 700B. The crushing or fracturing instrument 700B comprises a crushing or fracturing tip 710B coupled to or integrally formed with a crushing or fracturing shaft 705. The crushing or fracturing shaft 705 may be partially or fully solid or hollow (e.g., a generally cylindrical tubing), for example and without limitation, such as hollow distally near the crushing or fracturing tip lumen 760 (and solid proximally). The crushing or fracturing tip 710B comprises a plurality of sharp points 735 with beveled and chamfered inner edges 740 (chamfered toward the crushing or fracturing tip lumen 760) for significant sharpness and cutting or fracturing ability, with each sharp point 735 and at least two beveled and chamfered inner edges 740 forming a crushing or fracturing spear 755, with the crushing or fracturing spears 755 illustrated in FIGS. 33 and 34 being generally triangular. A crushing or fracturing instruments 700-700S may be configured to have any number or type of crushing or fracturing spears 755. In addition, the angle (“a”) 750 between each of the crushing or fracturing spears 755, may be varied, in addition to the illustrated 60° in FIG. 34, such as 30° or 45°, for example and without limitation. The crushing or fracturing tip 710B may have any number of sharp points 735 with beveled and chamfered inner edges 740, in addition to or fewer than the four sharp points 735 and eight beveled and chamfered inner edges 740 illustrated in FIGS. 33 and 34, also for example and without limitation. Not separately illustrated, the crushing or fracturing spears 755 of the crushing or fracturing instrument 700B may also each have an opening, relief or cut-out 745 (illustrated in FIGS. 35 and 35), providing a hole for clearing out any debris or debris build up, such as for aspiration, in addition to the crushing or fracturing tip lumen 760. The crushing or fracturing instruments 700A-700S may have any dimensions (lengths and diameters) suitable for insertion through an access sheath 100, 100A, 200, 200A, 500, 500A, 600, 600A, 800, 900 and into a subject gallbladder, or suitable for insertion coaxially within the shaft 835, 935 of a crushing or fracturing control apparatus 850, 950 which are then collectively inserted through an access sheath 100, 100A, 200, 200A, 500, 500A, 600, 600A, 800, 900 and into a subject gallbladder.

FIG. 35 is a partial isometric view illustrating a representative fourth embodiment of a crushing or fracturing instrument 700C. FIG. 36 is a partial, plan view illustrating the representative fourth embodiment of the crushing or fracturing instrument 700C. The crushing or fracturing instrument 700C comprises a crushing or fracturing tip 710C coupled to or integrally formed with a crushing or fracturing shaft 705. The crushing or fracturing shaft 705 also may be partially or fully solid or hollow (e.g., a generally cylindrical tubing), for example and without limitation, such as hollow distally near the crushing or fracturing tip lumen 760 (and solid proximally). The crushing or fracturing tip 710C comprises a plurality of sharp points 735A with beveled edges 740A, also for significant sharpness and cutting or fracturing ability, with each sharp point 735A and at least two beveled edges 740A forming a crushing or fracturing spear 755A, with the crushing or fracturing spears 755 illustrated in FIGS. 35 and 36 also being generally triangular.

Optionally, the beveled edges 740A may also be chamfered. The crushing or fracturing spears 755A of the crushing or fracturing instrument 700C further each have an opening, relief or cut-out 745, providing a hole for clearing out any debris or debris build up, such as for aspiration, in addition to the crushing or fracturing tip lumen 760.

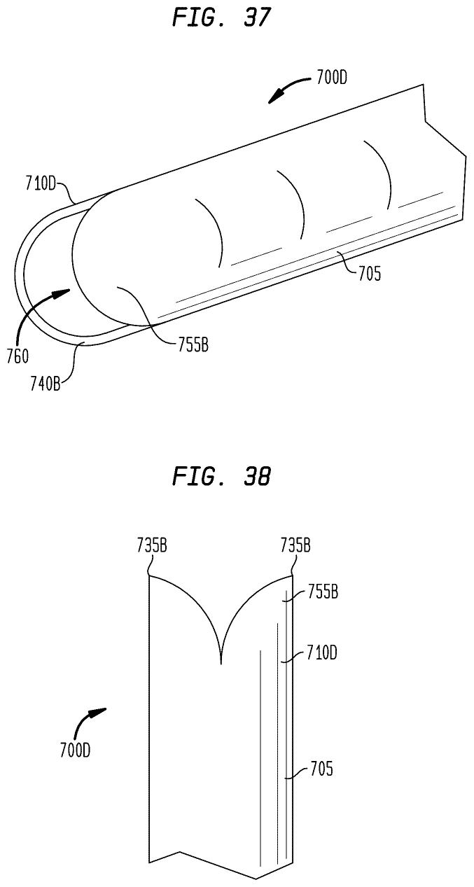

FIG. 37 is a partial isometric view illustrating a representative fifth embodiment of a crushing or fracturing instrument 700D. FIG. 38 is a partial, plan view illustrating the representative fifth embodiment of the crushing or fracturing instrument 700D. The crushing or fracturing instrument 700D comprises a crushing or fracturing tip 710D coupled to or integrally formed with a crushing or fracturing shaft 705. The crushing or fracturing shaft 705 also may be partially or fully solid or hollow (e.g., a generally cylindrical tubing), for example and without limitation, such as hollow distally near the crushing or fracturing tip lumen 760 (and solid proximally). The crushing or fracturing tip 710D comprises a plurality of sharp points 735B with curved beveled and chamfered edges 740B, also for significant sharpness and cutting or fracturing ability, with the crushing or fracturing spears 755B illustrated in FIGS. 37 and 38 being generally semicircular. Not separately illustrated, the crushing or fracturing spears 755B of the crushing or fracturing instrument 700D may also each have an opening, relief or cut-out 745, providing a hole for clearing out any debris or debris build up, such as for aspiration, in addition to the crushing or fracturing tip lumen 760.

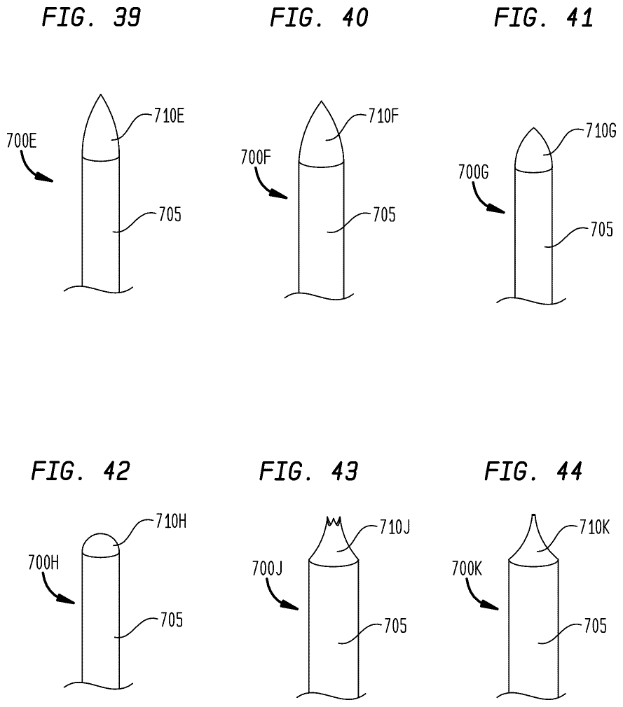

Additional crushing or fracturing tip 710E-710 S geometries are illustrated in FIGS. 39-51 for the corresponding sixth through eighteenth embodiments of a crushing or fracturing instrument 700E-700S. Each of the crushing or fracturing instruments 700E 700S also comprises a corresponding crushing or fracturing tip 710E-710S coupled to or integrally formed with a crushing or fracturing shaft 705. The crushing or fracturing instrument 700E includes a corresponding crushing or fracturing tip 710E having a first, narrow bullet configuration (e.g., a 50° tip angle), the crushing or fracturing instrument 700F includes a corresponding crushing or fracturing tip 710F having a second, wider bullet configuration (e.g., a 60° tip angle), while the crushing or fracturing instrument 700G includes a corresponding crushing or fracturing tip 710G having a third, further wider bullet configuration (e.g., 90° tip angle). The crushing or fracturing instrument 700H includes a corresponding crushing or fracturing tip 710H having a spherical or “button” configuration. The crushing or fracturing instrument 700J includes a corresponding crushing or fracturing tip 710J having a concave tri-tip configuration. The crushing or fracturing instrument 700K includes a corresponding crushing or fracturing tip 710K having a concave spear configuration. The crushing or fracturing instrument 700L includes a corresponding crushing or fracturing tip 710L having a core drilling configuration. The crushing or fracturing instrument 700M includes a corresponding crushing or fracturing tip 710M having a fluted configuration. The crushing or fracturing instrument 700N includes a corresponding crushing or fracturing tip 710N having a hollow configuration. The crushing or fracturing instrument 700P includes a corresponding crushing or fracturing tip 710P having a first, narrow spear configuration (e.g., a 30° tip angle), the crushing or fracturing instrument 700Q includes a corresponding crushing or fracturing tip 710Q having a second, somewhat wider spear configuration (e.g., a 45° tip angle), while the crushing or fracturing instrument 700R includes a corresponding crushing or fracturing tip 710R having a third, further wider spear configuration (e.g., 60° tip angle), and while the crushing or fracturing instrument 700S includes a corresponding crushing or fracturing tip 710S having a fourth, further wider spear configuration (e.g., 90° tip angle).

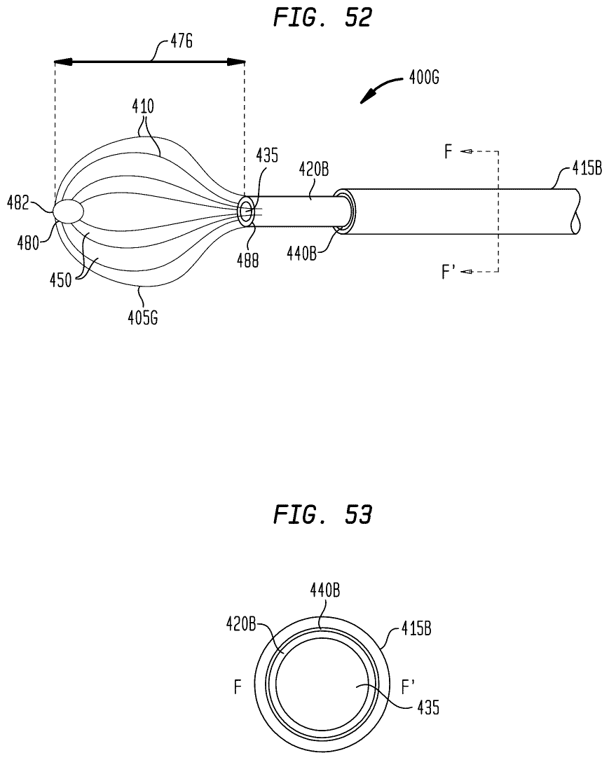

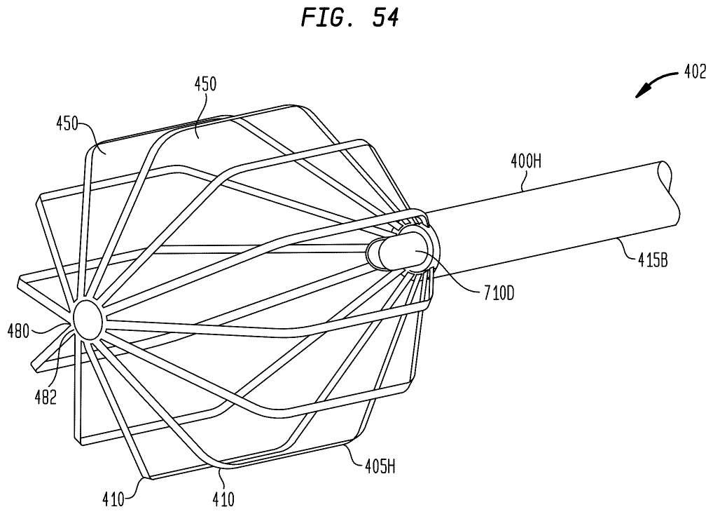

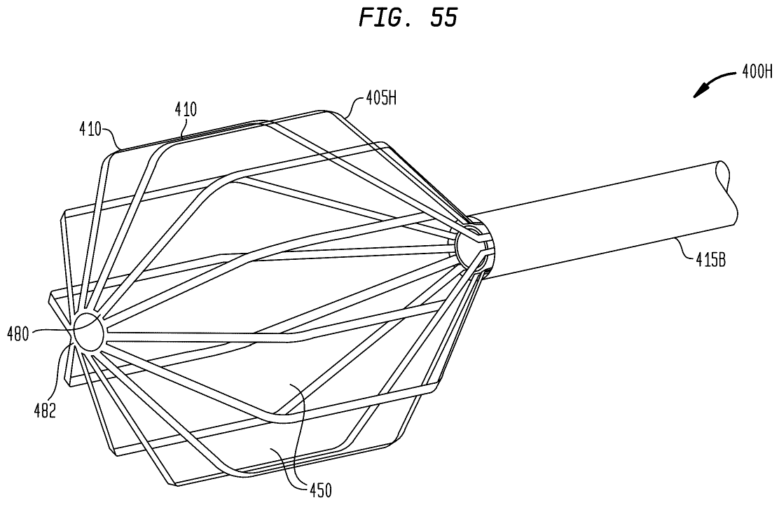

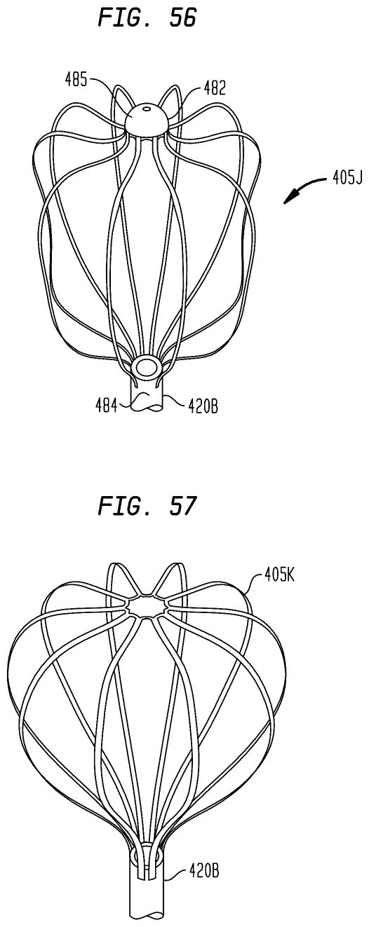

FIG. 52 is a partial isometric view illustrating a representative eighth embodiment of a catheter 400G apparatus having an eighth embodiment of a basket or cage 405G. FIG. 53 is a cross-sectional view (through the F-F′ plane of FIG. 52) illustrating the catheter shaft 415B and catheter shaft lumens 440B, 435 of the representative eighth embodiment of the catheter 400G apparatus. FIG. 54 is a partial isometric view illustrating a representative ninth embodiment of a catheter apparatus 400H having a ninth embodiment of a basket or cage 405H, and illustrating a crushing or fracturing tip 710D of a crushing or fracturing instrument 700D. FIG. 54 also illustrates a capturing and fracturing system 402 (for capturing and fracturing or crushing a gallstone), comprising a catheter apparatus 400G, 400H and a crushing or fracturing instrument 700-700S moveable longitudinally and rotatably within the catheter 400G, 400H. FIG. 55 is a partial isometric view illustrating the representative ninth embodiment of the catheter apparatus 400H having a partially retracted ninth embodiment of a basket or cage 405H. FIG. 56 is an isometric view illustrating a representative tenth embodiment of a basket or cage 405J. FIG. 57 is an isometric view illustrating a representative eleventh embodiment of a basket or cage 405K. The catheter apparatuses (or catheters) 400G, 400H function identically to each other, and differ only in the shape (or configuration) of the cage or basket 405G, 405H, 405J, 405K utilized with the catheters 400G, 400H. Additionally, the cages or baskets 405G, 405H, 405J, 405K function identically to each other, differing only in shape or configuration and the number of struts 410 of the cage or basket 405G, 405H, 405J, 405K, and also the use of an end cap 485 at the distal end 480, such that any of the cages or baskets 405G, 405H, 405J, 405K may be utilized interchangeably within or as part of a catheter 400G, 400H. Referring to FIG. 52, the cage or basket 405G has a generally ellipsoid or ovoid shape (or configuration). Referring to FIG. 55, the cage or basket 405G has a generally cylindrical shape (or configuration) centrally and conical distally and proximally. Referring to FIG. 56, the cage or basket 405J has a generally pumpkin shape (or configuration), a generally cylindrical shape (or configuration) centrally and with struts 410 becoming S-curved distally and proximally. In addition, the cage or basket 405J has an atraumatic, molded end cap 485 coupled to the strut ring 482 at the distal end 480. Referring to FIG. 57, the cage or basket 405K has a generally bulbous shape (or configuration), wider distally and narrower proximally.

Referring to FIGS. 52-57, a representative embodiment of a catheter apparatus (or catheter) 400G, 400H comprises an outer catheter tube (or outer catheter member) 415B having an outer catheter lumen 440B. An inner catheter manipulation shaft (or member) 420B is arranged coaxially (concentrically) within the outer catheter lumen 440B and is moveable longitudinally (or longitudinally and rotatably) (e.g., push-pull with or without rotation) within the outer catheter lumen 440B. The inner catheter manipulation shaft (or member) 420B has an inner catheter shaft lumen 435, as an option, for example and without limitation, configured and sized for insertion of a crushing or fracturing instrument 700A-700S. The struts 410 of the cage or basket 405G, 405H, 405J, 405K are coupled (such as through an adhesive, glue, or cement, for example and without limitation) to the outer periphery 484 of the inner catheter manipulation shaft 420B, and not to the outer catheter tube (or outer catheter member) 415B. The cage or basket 405G, 405H, 405J, 405K is retracted and compressed within the outer catheter lumen 440B while the catheter 400G, 400H is inserted through an access sheath 100, 100A, 200, 200A, 500, 500A, 600, 600A, 800, 900. When catheter 400G, 400H is positioned at a selected or desired location within the subject gallbladder, the inner catheter manipulation shaft (or member) 420B is extended (moved distally) relative to the outer catheter tube (or outer catheter member) 415B (or, equivalently, the outer catheter tube (or outer catheter member) 415B is retracted), resulting in the inner catheter manipulation shaft (or member) 420B being extended through the outer catheter lumen 440B and the cage or basket 405G, 405H, 405J, 405K is unsheathed, exposed from the outer catheter tube (or outer catheter member) 415B and expanded, as illustrated in FIGS. 52, 54-57.

The cage or basket 405G, 405H, 405J, 405K comprises a plurality of struts 410, with the plurality of struts 410 which are arranged longitudinally, and not in a mesh crossing pattern. The plurality of struts 410 of the cage or basket 405G, 405H, 405J, 405K have comparatively larger pores or openings 450 along the length (longitudinal orientation) of the cage or basket 405G, 405H, 405J, 405K. The plurality of struts 410 of the cage or basket 405G, 405H, 405J, 405K are coupled to or integrally formed with a strut ring 482 at the distal end 480, and are coupled at the proximal end 486 of the struts 410 to the outer periphery 484 of the inner catheter manipulation shaft 420B (at the distal end 488 of the inner catheter manipulation shaft 420B as illustrated). As described above, the plurality of struts 410 of the cage or basket 405G, 405H, 405J, 405K may be comprised of any suitable material (e.g., nitinol or other metals or metallic alloys, carbon fiber, or any of various suitable polymers).

The cage or basket 405G, 405H, 405J, 405K may be fabricated as known or becomes known in the catheter field. In a representative embodiment, not separately illustrated, the cage or basket 405G, 405H, 405J, 405K is laser cut from a nitinol tubing having an outer diameter in the range from 3.0 mm to 8 mm, and more particularly from 5.0 mm to 7.0 mm, and more particularly having an outer diameter of 6.0 mm, with the tubing having a wall thickness in the range from 0.22 mm to 0.5 mm, and more particularly from 0.22 mm to 0.4 mm, and more particularly having a tubing wall thickness of 0.22 mm. In a representative embodiment, the number of struts 410 of the basket or cage 405G, 405H, 405J, 405K which are laser cut may range from eight to sixteen struts 410, and more particularly from eight to twelve struts 410, and more particularly ten struts 410, for example and without limitation. In a representative embodiment, the length of the laser cut struts (prior to shaping) is in the range from 2.2 inches to 4.1 inches (55 mm to 104 mm), for use respectively with 25 mm to 50 mm heat set mandrels. In a representative embodiment, the width (or diameter) of each strut 410 of a cage or basket 405G, 405H, 405J, 405K may range from 0.25 mm to 1.0 mm, and more particularly from 0.25 mm to 0.7 mm, and more particularly having a strut 410 width or diameter of 0.5 mm, for example and without limitation. The laser cut tubing is then shaped and/or stretched using a heat set mandrel and electropolishes, as known in the field. In a representative embodiment, the shape set diameter of the cage or basket 405G, 405H, 405J, 405K may range from 10.0 mm to 60.0 mm, and more particularly from 20.0 mm to 50.0 mm, and more particularly from 25.0 mm to 40.0 mm, and more particularly having a diameter of the cage or basket 405G, 405H, 405J, 405K of 25.0 mm, for example and without limitation. In a representative embodiment, the resulting cage or basket 405G, 405H, 405J, 405K has a gap 450 between the struts 410 having a gap width 478 from 10.0 to 15.0 mm when fully expanded (unconstrained), for example and without limitation. In a representative embodiment, the resulting cage or basket 405G, 405H, 405J, 405K has an overall length having a gap width 478 from 10.0 to 15.0 mm when fully expanded (unconstrained), also for example and without limitation.

As illustrated, by moving the inner catheter manipulation shaft (or member) 420B longitudinally within the outer catheter lumen 440B, relative to the outer catheter tube or member 415B (or vice-versa), the cage or basket 405G, 405H, 405J, 405K may be expanded or contracted. For example, as the inner catheter manipulation shaft 420B is extended (moved distally), the distal end 480 of the cage or basket 405G, 405H, 405J, 405K is moved furthest away or furthest from the outer catheter tube or member 415B, the shape of the cage or basket 405G becomes more elongated (e.g., fusiform or spindle-shaped) and the diameter of the cage or basket 405G is enlarged or expanded, for capture of any gallstones or gallstone fragments, with similar expansion of the diameter of the cages or baskets 405H, 405J, 405K as the inner catheter manipulation shaft 420B is extended (moved distally relative to the outer catheter tube or member 415B). As the basket or cage 405G, 405H, 405J, 405K is pressed against a gallstone, the struts 410 deflect to allow the stone to squeeze through the strut gaps 450, then return to their original configuration, trapping the stone within the basket or cage 405G, 405H, 405J, 405K. As the outer catheter tube or member 415B is advanced, or equivalently the inner catheter manipulation shaft 420B is retracted into the outer catheter tube or member 415B, the distal end 480 of the cage or basket 405G, 405H, 405J, 405K is moved closer to the outer catheter tube or member 415B, the struts 410 of the cage or basket 405G, 405H, 405J, 405K are retracted with the inner catheter manipulation shaft 420B into the outer catheter lumen 440B of the outer catheter tube or member 415B, the remaining length 476 of the cage or basket 405G, 405H, 405J, 405K is shortened and the basket or cage 405G, 405H, 405J, 405K is contracted around any captured gallstones or gallstone fragments, such as illustrated in FIG. 30B.

The catheter apparatus 400F illustrated in FIGS. 30A and 30B operates and functions similarly to the catheter apparatuses 400G, 400H. As illustrated, a gallstone 411 may be captured through the gaps in the cage or basket 405F. The inner catheter manipulation shaft 420, 420A is retracted into the outer catheter tube or member 415B, the distal end of the cage or basket 405F is moved closer to the outer catheter tube or member 415B, the struts 410 of the cage or basket 405F are retracted with the inner catheter manipulation shaft 420, 420A into the outer catheter lumen 440, 440A of the outer catheter tube or member 415B, and the basket or cage 405F is contracted around any captured gallstones 411 or gallstone fragments. The crushing or fracturing instrument 700-700S may then be advanced into the gallstone 411 or gallstone fragments to create fracturing or crushing, as illustrated in FIG. 30B. The catheter apparatus 400F differs insofar as the crushing or fracturing instruments 700-700S are insertable in parallel with the inner catheter manipulation shaft 420, 420A within the outer catheter lumen 440, 440A of the outer catheter tube or member 415B, rather than coaxially or concentrically, as illustrated in FIGS. 30A, 30B.

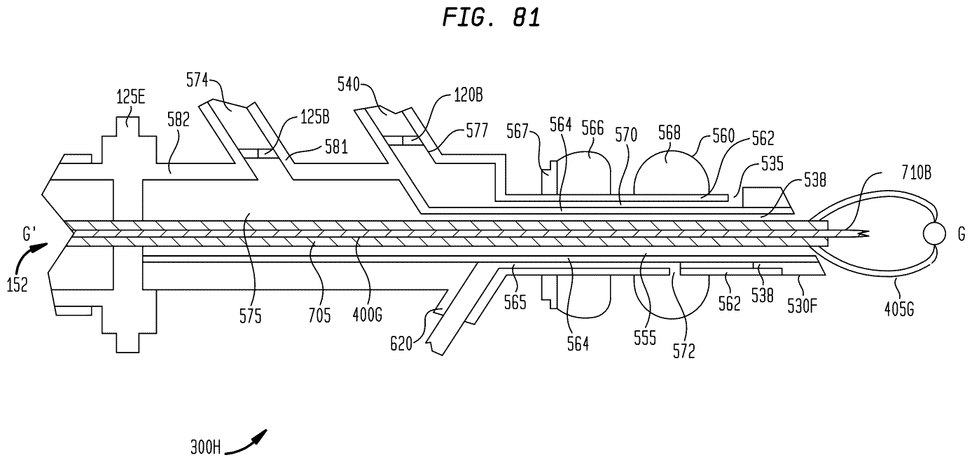

Depending upon whether the catheter apparatus 400F, 400G, 400H is implemented to be flexible or rigid (stiff), the catheter apparatus 400F, 400G, 400H may be inserted through the access ports 130, 130A, 135, 620 of the access sheath 100, 100A, 200, 200A, 500, 500A, 600, 600A, 800, 900 or in-line, directly through the lumen 165, 555 of a tubular shaft 105, 505, 505A of an access sheath 100, 100A, 200, 200A, 500, 500A, 600, 600A, 800, 900, such as in-line, directly through the tubular shaft 505, 505A of an access sheath 800, 900 (illustrated in FIG. 81). Once the cage or basket 405F, 405G, 405H, 405J, 405K is expanded inside the gallbladder, the cage or basket 405F, 405G, 405H, 405J, 405K may be manipulated (rotated, advanced, retracted, expanded, etc.) to capture gallstones within the plurality of struts 410 of the cage or basket 405G, 405H, 405J, 405K. The user could then tighten (contract) the cage or basket 405F, 405G, 405H, 405J, 405K around the gallstones for removal, fracturing, or crushing. For gallstones or gallstone fragments having a size smaller than the inner diameter of the lumen 165 of the access sheath 100, 100A, 200, 200A or central lumen 555 of an access sheath 500, 500A, 600, 600A, 800, 900, the catheter apparatus 400F, 400G, 400H having the captured gallstone(s) or gallstone fragments may be removed from the access sheath 100, 100A, 200, 200A, 500, 500A, 600, 600A, 800, 900. As the cage or basket 405F, 405G, 405H, 405J, 405K is contracted, as described above and as described in greater detail below, the captured gallstone also may be fractured or crushed as well, reducing the sizes of the gallstone pieces to facilitate removal.

In representative embodiments, the inner catheter manipulation shaft 420B is hollow and has an inner catheter shaft lumen 435, useful for insertion of any of the various crushing or fracturing instruments 700-700S, especially a crushing or fracturing instrument 700A-700S, for example and without limitation. For example, when a gallstone has been captured, a crushing or fracturing instrument 700-700S may be inserted through the inner catheter shaft lumen 435 and utilized to fracture the gallstone. Also for example, as described above, when the catheter 400G, 400H has been inserted through an access sheath 100, 100A, 200, 200A, 500, 500A, 600, 600A, 800, 900 and positioned at a selected or desired location within the subject gallbladder, following capture of a gallstone, the basket or cage 405G, 405H, 405J, 405K may be retracted around the gallstone, the crushing or fracturing instrument 700-700S may be inserted through the inner catheter shaft lumen 435 and positioned near or abutting the captured gallstone, and the crushing or fracturing instrument 700-700S may be actuated, resulting in the angled or pointed crushing or fracturing tip 710 -710S impacting the gallstone captured in the cage or basket 405G, 405H, 405J, 405K, to fracture the gallstone. The fractured gallstone or gallstone fragment may then be removed from the subject gallbladder using the catheter 400G, 400H or by aspiration, for example and without limitation. The crushing or fracturing instruments 700-700S may also be utilized with the other catheters 400-400F having other cages or baskets 405-405F, including those without an inner catheter shaft lumen 435, as described above for the catheter apparatus 400F.

In a representative embodiment, the basket or cage 405G, 405H, 405J, 405K has an outer diameter in the range of 25-50 mm in the unsheathed, expanded state, for example and without limitation. Also in a representative embodiment, the outer catheter tube (or outer catheter member) 415B has a length in the range of 30 cm, an outer diameter in the range of 8.5 mm, and an inner diameter in the range of 6.2 mm, for example and without limitation. Also in a representative embodiment, the inner catheter manipulation shaft 420B has a length in the range of 41 cm, for example and without limitation. When used in conjunction with the catheter 400G, 400H, the crushing or fracturing instrument 700-700S is configured and sized accordingly. In a representative embodiment, for example and without limitation, the crushing or fracturing instrument 700-700S has a shaft 705 outer diameter in the range of 5.6 mm and a length in the range of 55.9 cm, with the crushing or fracturing instrument 700-700S having an overall length in the range of 73.2 cm. In another representative embodiment, for example and without limitation, the crushing or fracturing instrument 700-700S has a shaft 705 outer diameter in the range from 0.156 inches to 0.167 inches (3.96 mm to 4.24 mm), and if hollow, an inner diameter in the range of 0.097 inches to 0.116 inches (2.46 mm to 2.94 mm), and if partially hollow (distally) extending from the distal end for 0.3 inches (7.62 mm), with the crushing or fracturing instrument 700-700S having an overall length in the range of 21 inches (53.4 cm).

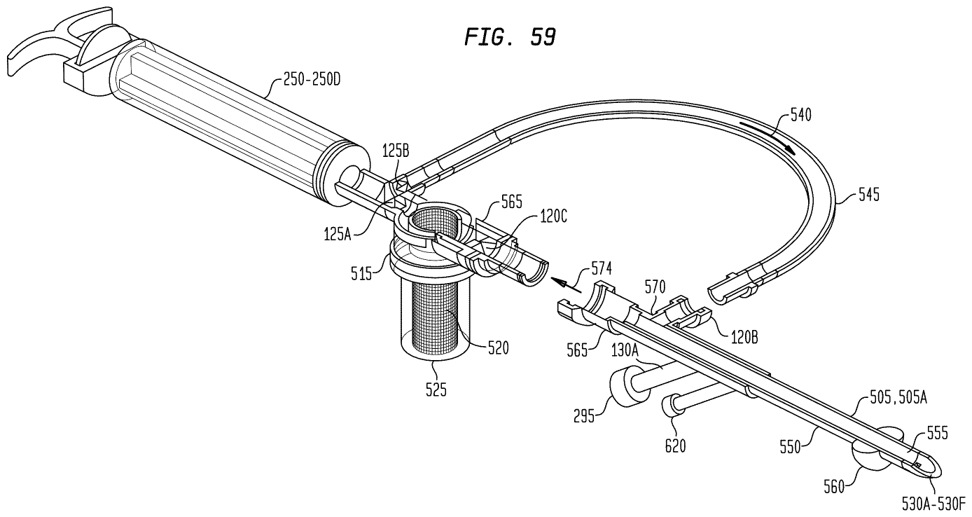

FIG. 58 is an isometric, partially exploded view illustrating a representative seventh embodiment of a system 300F having the fifth and sixth embodiments of an access sheath 500, 500A and any of the representative embodiments of an aspiration device 250 250D. FIG. 59 is a partial isometric and cut-away view illustrating the representative seventh embodiment of the system 300F having the fifth and sixth embodiments of the access sheath 500, 500A and any of the representative embodiments of an aspiration device 250-250D.

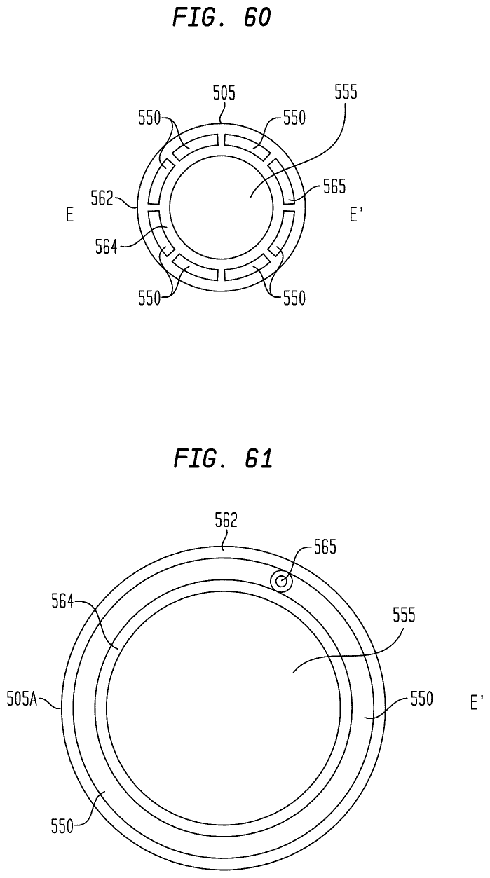

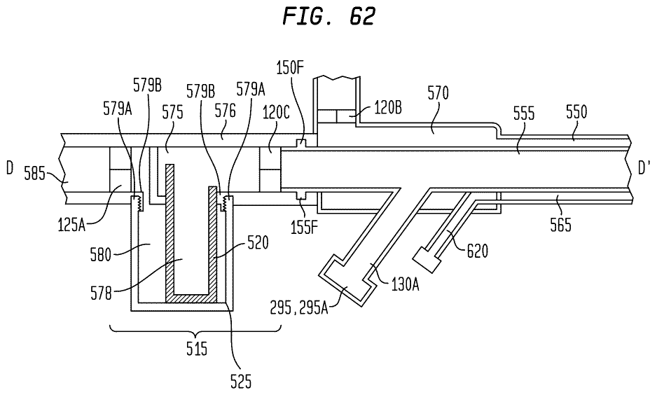

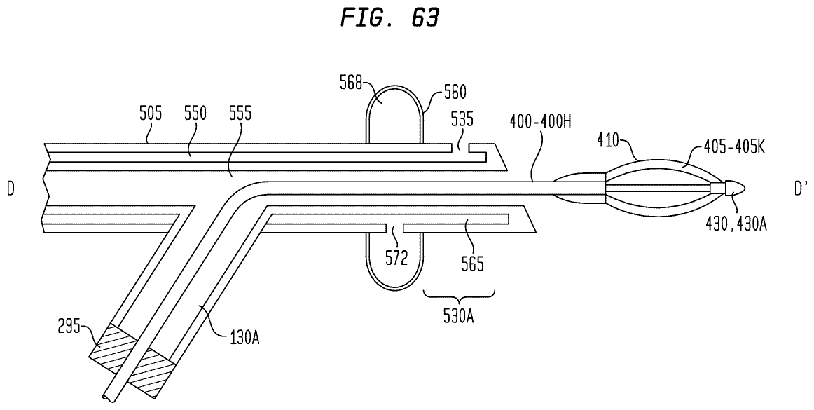

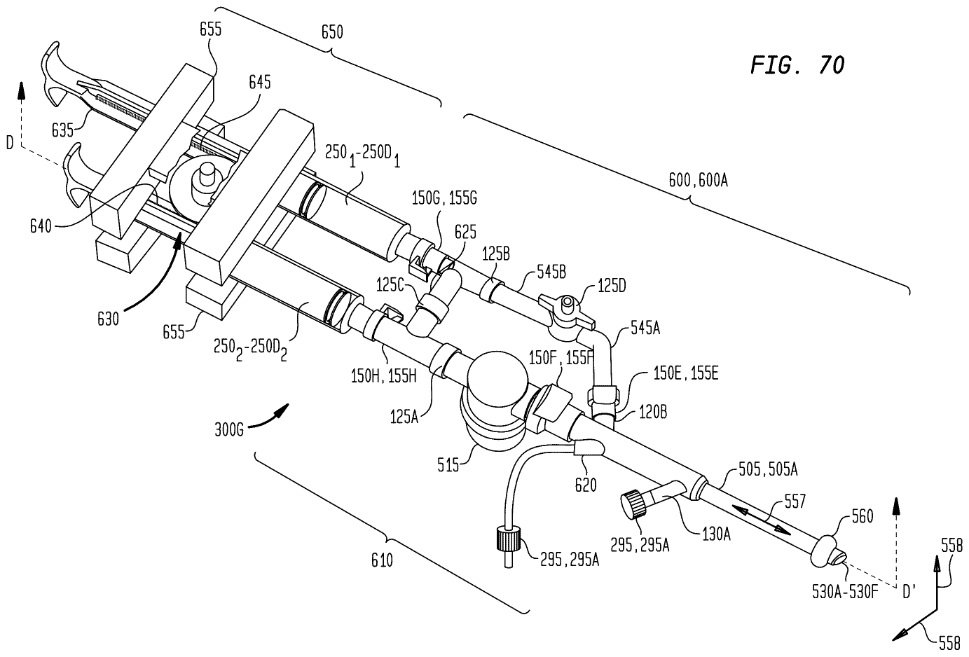

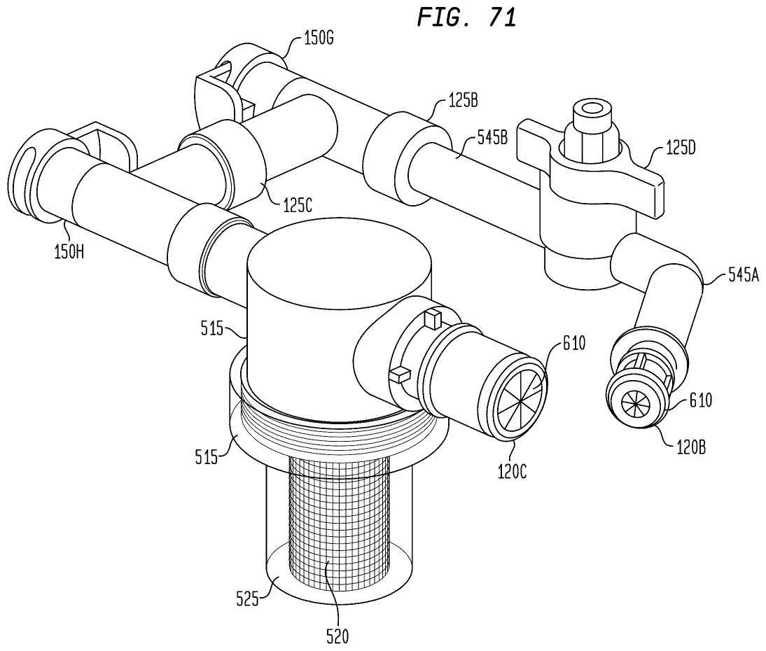

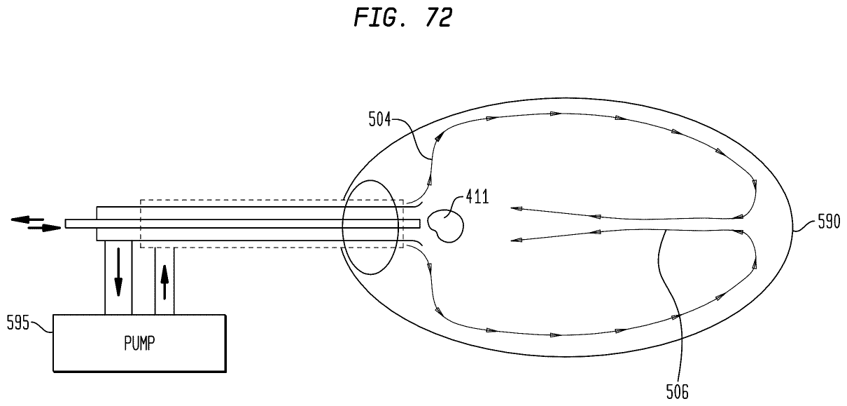

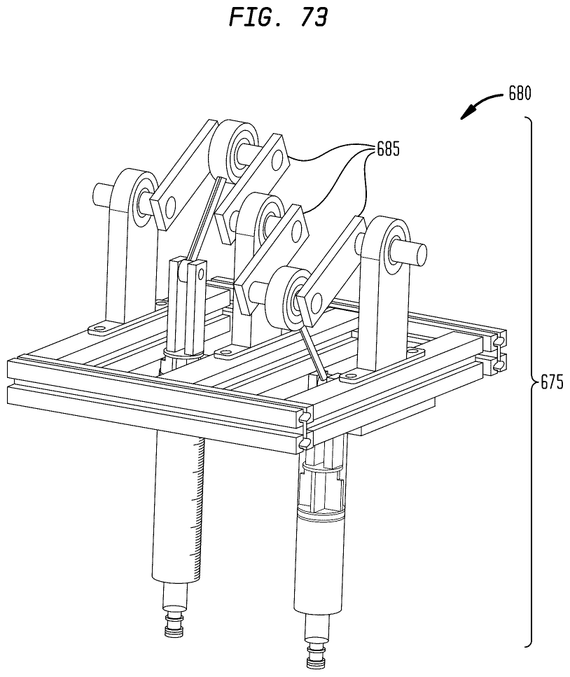

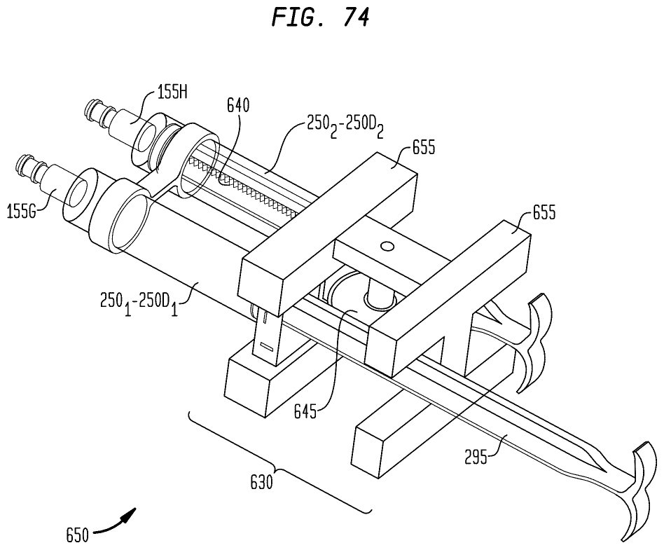

FIG. 60 is a first cross-sectional view (through the E-E′ plane of FIG. 58) of a first tubular shaft 505 of the fifth embodiment of the access sheath 500 of the representative seventh embodiment of the system 300F. FIG. 61 is a second cross-sectional view (through the E-E′ plane of FIG. 58) illustrating a second tubular shaft 505A of the representative sixth embodiment of the access sheath 500A of the representative seventh embodiment of the system. FIG. 62 is a partial cross-sectional view (through the D-D′ plane of FIGS. 58 and 70) of the proximal hub 510, 610 of the respective access sheaths 500, 500A, 600, 600A of the representative systems 300F, 300G. FIG. 63 is a partial cross-sectional view (through the D-D′ plane of FIGS. 58 and 70) illustrating the representative access sheaths 500, 500A, 600, 600A of the representative systems 300F, 300G, additionally with a representative catheter 400-400H embodiment.

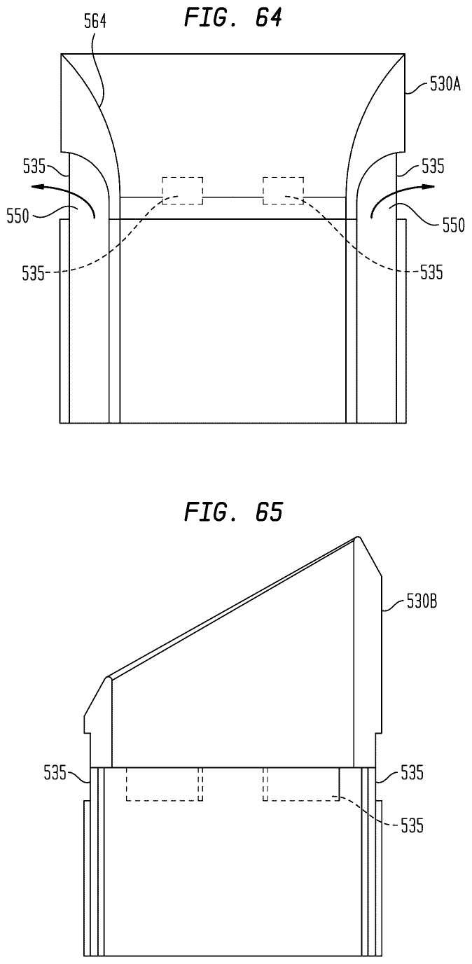

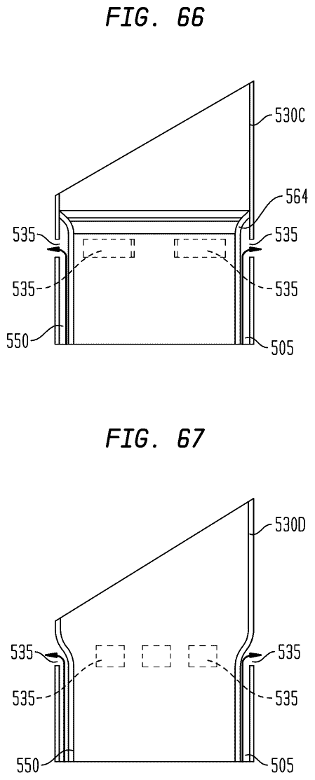

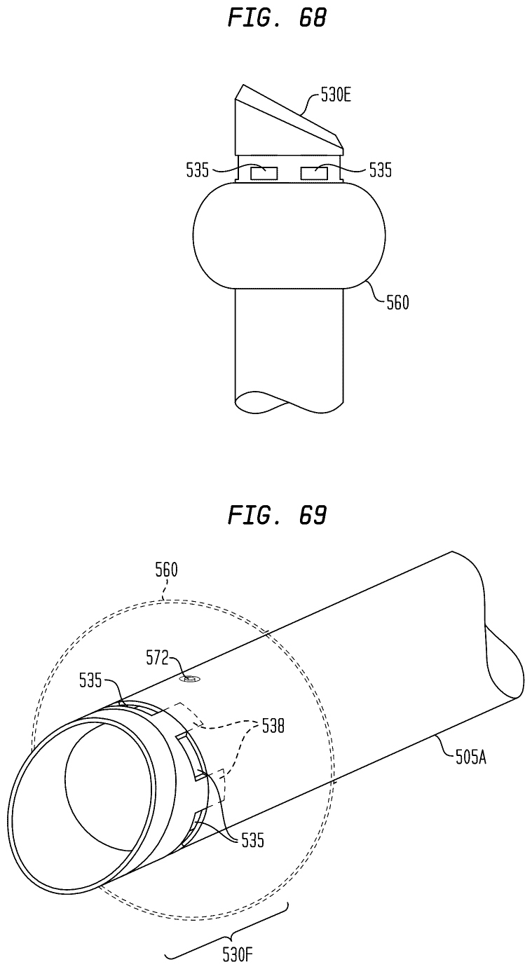

FIG. 64 is a side, elevational view illustrating a representative first embodiment of an access sheath tip 530A. FIG. 65 is a side, elevational view illustrating a representative second embodiment of an access sheath tip 530B. FIG. 66 is an isometric, cut-away view illustrating a representative third embodiment of an access sheath tip 530C. FIG. 67 is an isometric, cut-away view illustrating a representative fourth embodiment of an access sheath tip 530D. FIG. 68 is an isometric view illustrating a representative fifth embodiment of an access sheath tip 530E and an inflatable, sealing and anchoring balloon 560 of any of the various embodiments of an access sheath 500, 500A, 600, 600A, 800, 900. FIG. 69 is an isometric, cut-away view illustrating a representative sixth embodiment of an access sheath tip 530F of the second tubular shaft 505A of the representative access sheaths 500A, 600A, 800.

FIG. 70 is an isometric view illustrating a representative eighth embodiment of a system 300G having seventh and eighth embodiments of an access sheath 600, 600A and a first embodiment of a dual aspiration and infusion assembly 650. FIG. 71 is a partial, isometric view illustrating a representative proximal hub 610 of the representative eighth system 300G embodiment having the seventh and eighth embodiments of an access sheath 600, 600A. FIG. 72 is a diagram illustrating fluid flow in a representative gallbladder 590 using the seventh and/or eighth system 300F, 300G embodiments. FIG. 73 is an isometric view illustrating a representative second embodiment of a dual aspiration and infusion assembly 675 which may be utilized as part of the representative eighth system 300G embodiment.

Referring to FIGS. 58-73, the system 300F for gallstone removal comprises several components, including an access sheath 500, 500A and any aspiration device 250 250D, and the system 300G for gallstone removal comprises several components, including an access sheath 600, 600A and a dual aspiration and infusion assembly 650, 675. Each of the systems 300F, 300G may further comprise a catheter 400-400H. More specifically, in a representative embodiment, a system 300F comprises an access sheath 500, 500A coupled through a proximal hub 510 to an aspiration device 250-250D, such as illustrated in FIGS. 58 and 59. Also more specifically, in a representative embodiment, a system 300G comprises an access sheath 600, 600A coupled through a proximal hub 610 to a dual aspiration and infusion assembly 650, 675, such as illustrated in FIGS. 70, 71 and 73. As with the various systems described above, the systems 300F, 300G may further include a catheter 400-400H, such as illustrated in FIG. 63, arranged through one or more access ports 130A. Apart from the differences described in greater detail below, the systems 300F, 300G function similarly to the other systems 300-300E described above. The notable differences of the systems 300F, 300G are the inclusion of multiple, separate infusion and aspiration channels 540, 574, respectively, the inclusion of a filter assembly 515 in the proximal hub 510, 610 as part of the aspiration channel 574, and the optional inclusion of a sealing and anchoring balloon 560, which further utilizes a separate inflation lumen 565. The sealing and anchoring balloon 560 may be comprised of any suitable expandable material, such as medical/surgical grade Pebax or nylon, for example and without limitation.

The access sheath 500 differs from the access sheath 500A solely insofar as the access sheath 500 includes a tubular shaft 505, while the access sheath 500A includes a tubular shaft 505A instead. The access sheath 600 differs from the access sheath 600A solely insofar as the access sheath 600 includes a tubular shaft 505, while the access sheath 600A includes a tubular shaft 505A instead. The tubular shafts 505, 505A differ from each other concerning the configuration of the peripheral lumen(s) 550 and the inflation lumen 565, and otherwise function identically to each other. Unless the context indicates otherwise, reference to any access sheath 500, 500A, 600, 600A will be understood to mean and include any of the access sheaths 500, 500A, 600, 600A.

The access sheath 500 (or 500A) comprises a cylindrical, hollow, tubular shaft or member 505 (or 505A) coupled to a proximal hub 510. The access sheath 600 (or 600A) comprises a cylindrical, hollow, tubular shaft or member 505 (or 505A) coupled to a proximal hub 610. The tubular shaft 505, 505A is comprised of a first, outer shaft wall 562 and a second, inner shaft wall 564, which collectively form multiple and separate interior lumens: a central lumen 555 arranged interiorly or centrally to the second, inner shaft wall 564 as illustrated, and one or more peripheral lumens 550 arranged between the first, outer shaft wall 562 and a second, inner shaft wall 564 (and arranged peripherally to the central lumen 555, and may also further be arranged spaced apart about the periphery of the tubular shaft 505, as illustrated in FIG. 60). The access sheath 500, 500A, 600, 600A may also include an optional inflation lumen 565 (when an optional sealing and anchoring balloon 560 is included as part of the access sheath 500, 500A, 600, 600A), also formed by the first, outer shaft wall 562 and a second, inner shaft wall 564, and also arranged peripherally to the central lumen 555. The central lumen 555 extends from the proximal hub 510 and through the sheath tip 530A-530F at the distal end 140A of the access sheath 500, 500A, 600, 600A. Each peripheral lumen 550 extends from the proximal hub 510 to a corresponding infusion port 535, with the corresponding one or more infusion ports 535 arranged spaced apart in the sheath tip 530A-530F and further spaced apart proximally from the distal end 140A of the access sheath 500, 500A, 600, 600A. As a result of this novel structure, the infusing fluid exits the tubular shaft 505, 505A of the access sheath 500, 500A, 600, 600A (via the peripheral lumen(s) 550 and infusion ports 535) radially (504) from the outer periphery of the access sheath 500, 500A, 600, 600A, i.e., generally normal or perpendicular to the longitudinal dimension (502) of the access sheath 500, 500A, 600, 600A. In representative embodiments, for example and without limitation, an access sheath 500, 500A, 600, 600A may be 15 cm (150 mm) long, with the central lumen 555 having an inner diameter of 8-10 mm (24-30 Fr), with the tubular shaft 505 comprising stainless steel.

As illustrated in FIG. 60, the tubular shaft 505 includes an inflation lumen 565 and a plurality of peripheral lumens 550 between the first, outer shaft wall 562 and the second, inner shaft wall 564, with each peripheral lumen 550 extending to a separate, corresponding infusion port 535. Access sheaths 500A, 600A differ from access sheaths 500, 500A, 600, 600A, insofar as the tubular shaft 505A includes an inflation lumen 565 and a single, large peripheral lumen 550 between the first, outer shaft wall 562 and the second, inner shaft wall 564, as illustrated in FIG. 61, with the single peripheral lumen 550 extending to a plurality of infusion ports 535. As illustrated in FIG. 69, the access sheath tip 530F includes a plurality of tabs 538 extending between the first, outer shaft wall 562 and the second, inner shaft wall 564, with the plurality of infusion ports 535 spaced apart peripherally in between the plurality of tabs 538, also providing that the infusing fluid exits the tubular shaft 505A of the access sheath 500A, 600A (via the peripheral lumen 550 and infusion ports 535) radially (504) from the outer periphery of the access sheath 500A, 600A. Apart from having these specific structural differences, the tubular shafts 505, 505A function identically to each other for infusion, inflation, and aspiration.

As previously described, the distal portion of the tubular shaft 505 of the access sheath 500, 500A, 600, 600A may be inserted into a subject gallbladder 590 (illustrated schematically in FIG. 72) for removal of one or more gallstones and gallstone fragments. The central lumen 555 forms part of the aspiration channel 574, as described in greater detail below, for aspiration of fluid, gallstone(s), and gallstone fragments from the subject gallbladder through the central lumen 555 and into the filter assembly 515 of the proximal hub 510. The peripheral lumen(s) 550 form part of the infusion channel 540, also as described in greater detail below, for infusion of fluid radially (504) into the subject gallbladder (590).

In a representative embodiment, the proximal hub 510 comprises a housing 576 having a plurality of hub lumens 570, 575, 585, a first port 577; a filter assembly 515 having a filter chamber 578; a plurality of valves 120B, 120C, 125A, 125B coupled within or to the housing 576; infusion tubing 545 (coupled to the first port 577 and in fluid communication with the peripheral lumens 550), and a plurality of mating couplings 150E and 155E, 150F and 155F, and coupling 150D to mate with coupling 155D of the aspiration device 250, 250A, 250B, 250C, or 250D. Mating couplings 150D and 155D removably couple a (first) aspiration device 250, 250A, 250B, 250C, or 250D to the proximal hub 510. Mating couplings 150E and 155E connect the infusion tubing 545 to the hub lumen 570 through the first port 577, which in turn is in fluid communication with the peripheral lumens 550. Mating couplings 150F and 155F connect the tubular shaft 505 to the proximal hub 510. The valves 120B, 120C, 125A, 125B are typically each one-way valves, such as stop cock valves or pressure cracking valves (illustrated in FIG. 71 as pressure cracking valves 610), may each be implemented as previously described. Also, the mating couplings 150D and 155D, 150E and 155E, 150F and 155F may each be implemented as previously described, such as quick connect fittings, screw threads, snap fittings, etc. It should also be noted that the coupling 150E may be incorporated into or be a part of the first port 577.

The filter assembly 515 comprises a filter 520 forming a filter chamber 578, and a filter canister 525 removably coupleable to the housing 576, such as through mating screw threads 579A, 579B, with the filter canister 525 surrounding the filter 520, and forming a filter canister lumen 580 around the exterior of the filter 520, i.e., the filter canister lumen 580 is on the side of the filter opposite from the filter chamber 578.

The hub lumen 570 is arranged between the infusion tubing 545 and the peripheral lumens 550. The valve 120C is arranged between the central lumen 555 of the tubular shaft 505 and the hub lumen 575. The hub lumen 575 is arranged between the valve 120C and the filter 520, as part of the filter assembly 515. The filter chamber 578 is the interior portion of the filter 520, and is in fluid communication with the hub lumen 575, forming the space within the filter 520 for filtering out and trapping any aspirated gallstone(s) or gallstone fragments. The filter canister lumen 580 is within the filter canister 525, on the side of the filter 520 opposite the filter chamber 578. The one-way valve 125A separates the filter canister lumen 580 from the hub lumen 585, and serves to prevent injected infusion fluid from entering the filter canister lumen 580 and the balance of the aspiration channel 574.

The proximal hub 610 includes the components and structures of the proximal hub 510 described above, and further includes additional couplings, such as to connect to a first aspiration device 250₁, 250A₁, 250B₁, 250C₁, or 250D₁ and also to a second aspiration device 250₂, 250A₂, 250B₂, 250C₂, or 250D₂ of the dual aspiration and infusion assembly 650, 675, additional valves to direct fluid flow within the proximal hub 610, and additional tubing, as described in greater detail below.

In various embodiments, the proximal hub 510, 610 may also comprise an access port 130A having a valve 295, 295A, such as for insertion of a catheter 400-400H, as described in greater detail below. Also in various embodiments, when an optional sealing and anchoring balloon 560 is included in the system 300F, 300G, the proximal hub 510 (610) may also include an inflation access port 620 in fluid communication with the separate inflation lumen 565, to infuse or aspirate fluid such as saline to respectively inflate and deflate the sealing and anchoring balloon 560.

In operation of the systems 300F, 300G, fluid is typically infused into the subject gallbladder to generate some turbulence to stir up any gallstone(s) or gallstone fragments for aspiration, as described in greater detail below. For infusion of fluid in the infusion channel 540, fluid from an aspiration device 250, 250A, 250B, 250C, 250D of system 300F or first aspiration device 250₁, 250A₁, 250B₁, 250C₁, or 250D₁ of system 300G is injected through hub lumen 585 (using a plunger 265 of the aspiration device 250, 250A, 250B, 250C, 250D or first aspiration device 250₁, 250A₁, 250B₁, 250C₁, or 250D₁) and through one-way valve 125B and into the infusion tubing 545, 545A. In turn, the fluid from the infusion tubing 545 flows through valve 120B (e.g., a pressure cracking valve 610) and into the hub lumen 570, which is in fluid communication with each of the peripheral lumens 550. The infusing fluid then flows through each of the peripheral lumens 550 and radially outward through each of the corresponding infusion ports 535, as mentioned above.

The filter assembly 515 of the proximal hub 510, 610 forms part of the aspiration channel 574. The filter assembly 515 comprises a filter 520 arranged within a filter canister 525. The filter 520 may be comprised of any suitable material, and typically has a sufficiently fine mesh to prevent gallstone(s) or gallstone fragments from passing through the filter 520. Both of the filter 520 and filter canister 525 are typically removably coupleable to the housing 576, such as to empty any gallstone(s) or gallstone fragments from the filter assembly 515. Any fluid, gallstone(s), and gallstone fragments aspirated from the subject gallbladder through the central lumen 555 passes through the valve 120C, typically implemented as a pressure cracking valve 610, and into the hub lumen 575 and the filter chamber 578 formed by the filter 520. Any aspirated gallstone(s) or gallstone fragments remain in the filter chamber 578 and do not pass through the filter 520, and further are prevented from exiting back into the central lumen 555 by the valve 120C. Any remaining fluid flows through the filter 520 and into the filter canister lumen 580, then through the (one-way) valve 125A into the hub lumen 585 and into the aspiration device 250, 250A, 250B, 250C, 250D of system 300F or the second aspiration device 250₂, 250A₂, 250B₂, 250C₂, or 250D₂ of system 300G.

As illustrated in FIG. 63, any of the various catheters 400-400H and/or crushing or fracturing instruments 700-700S may also be utilized with the access sheath 500, 500A, 600, 600A of the system 300F. A catheter 400-400H may be inserted through a valve 295, 295A and into the catheter access port 130A, through the central lumen 555, and out the distal end 140A of the access sheath 500 into the subject gallbladder. Alternatively, a catheter 400-400H or crushing or fracturing instrument 700-700S may be inserted in line (linearly aligned), through the hub lumens 585, 575 and through the central lumen 555 (such as illustrated in FIG. 81). Any gallstone capture, crushing, and removal may then be performed using the catheter 400-400H and/or crushing or fracturing instruments 700 700S, including in conjunction with the infusion and aspiration described above and further described below. In the event that the gallstone(s) and/or gallstone fragments may be larger than the inner diameter of the central lumen 555, using a catheter 400-400H and/or crushing or fracturing instruments 700-700S, the gallstone(s) and/or gallstone fragments may be captured and crushed prior to aspiration, and then the resulting, comparatively smaller gallstone(s) and/or gallstone fragments may then be aspirated through the central lumen 555 and aspiration channel 574 as described above.

Any of the various catheters 400-400H and/or crushing or fracturing instruments 700-700S may be utilized with the systems 300F, 300G. In a representative embodiment, a crushing or fracturing instrument 700-700S is utilized with a catheter 400G, 400H. In addition, as an option, the catheters 400-400E utilized may also have the appropriate proximal fluid connections to also be compatible with the systems 300F, 300G to perform aspiration through the catheters 400-400E (when the catheter 400-400E is implemented with a hollow shaft, as described above) while still infusing or irrigating through the peripheral lumens 550 as described above.

It may be difficult to target and position a device in order to perform a successful gallstone removal procedure. The systems 300F, 300G solve this problem by turbulently infusing the gallbladder, which tends to move any gallstone(s) or gallstone fragments toward the distal end 140A of the access sheath 500, 500A, 600, 600A for aspiration into the central lumen 555. Additionally, given the malleability of the gallbladder as a structure, this infusion of fluid also serves to help prevent its collapse, also a distinct advantage of the systems 300F, 300G.

In addition to the illustrated single aspiration device 250, 250A, 250B, 250C, 250D illustrated in FIGS. 58 and 59, an infusion source also can be comprised of a dual syringe (e.g., first aspiration device 250₁, 250A₁, 250B₁, 250C₁, or 250D₁ and second aspiration device 250₂, 250A₂, 250B₂, 250C₂, or 250D₂ of system 300G, illustrated and described below with reference to FIGS. 70 and 71), or a pump 680 as described below. The infusion source and filter assembly 515 can be disconnected from the access sheath 500, 500A, 600, 600A during portions of the procedure when stone removal is not taking place, such as initial access, or stone crushing sequences. Additionally, the in-line filter 520 can have a removable filter canister 525 so that the stones can be extracted from the system 300F, 300G and then the filter assembly 515 re-assembled for additional use. The check valves 125A, 125B direct flow such that aspiration is performed through the larger central lumen 555 and infusion is performed through the smaller peripheral lumens 550.

One aspect of the mechanism for removing gallstone(s) or gallstone fragments is driven by the multi-lumen access sheath tip 530A-530F. Referring to FIGS. 64-69 , a variety of access sheath tips 530A-530F are illustrated, any of which may be utilized in any of the various systems 300F, 300G, and any and all of which are considered equivalent and within the scope of the disclosure. For the access sheath tip 530A illustrated in FIG. 64, the inner wall 564 has a convex taper toward the distal end 140A of the access sheath 500 (i.e., a convex inner diameter). For the access sheath tip 530B illustrated in FIG. 65, the access sheath tip 530B is tapered or beveled, with a double-bevel, toward the distal end 140A of the access sheath 500 while maintaining a generally constant inner diameter of the central lumen 555. For the access sheath tip 530C illustrated in FIG. 66, the access sheath tip 530C is also tapered or beveled toward the distal end 140A of the access sheath 500, while maintaining generally constant inner diameter of the central lumen 555 and constant outer diameter of the tubular shaft 505 (and additionally with a removeable inner wall 564). For the access sheath tip 530D illustrated in FIG. 67, the access sheath tip 530D is also tapered or beveled toward the distal end 140A of the access sheath 500, while maintaining generally constant inner diameter of the central lumen 555 and constant outer diameter of the tubular shaft 505. For the access sheath tip 530E illustrated in FIG. 68, the access sheath tip 530E is also tapered or beveled toward the distal end 140A of the access sheath 500, but is shown having a square or rectangular configuration, rather than circular or cylindrical. For the access sheath tip 530F illustrated in FIG. 69, as described above, the access sheath tip 530F includes a plurality of tabs 538 extending between the first, outer shaft wall 562 and the second, inner shaft wall 564, with the plurality of infusion ports 535 spaced apart peripherally in between the plurality of tabs 538. The access sheath tip 530F is also tapered or beveled at an angle toward the distal end 140A of the access sheath 500, with a concave radial curvature, while maintaining a generally constant inner diameter of the central lumen 555 and constant outer diameter of the tubular shaft 505.

Also referring to FIG. 68, in another embodiment, the tubular shaft 505 may also include an inflatable, sealing and anchoring balloon 560 or other expandable structure, typically arranged proximally to the distal end 140A of the access sheath 500, 500A, 600, 600A, as illustrated. The distal end 140A of the tubular shaft 505, 505A of the access sheath 500, 500A, 600, 600A having the inflatable, sealing and anchoring balloon 560 or expandable structure is inserted into the subject gallbladder and the inflatable, sealing and anchoring balloon 560 is inflated, via the inflation lumen 565 and inflation port 572, typically by infusing saline or another liquid (or air) into the interior 568 of the inflatable, sealing and anchoring balloon 560, for example and without limitation, such as through inflation access port 620. This allows the distal end 140A of the tubular shaft 505 of the access sheath 500 to anchor inside of the gallbladder, and the inflatable, sealing and anchoring balloon 560 helps to create a fluid seal against the gallbladder wall. This creates a closed system for the cyclical irrigation and aspiration to be more effective. For example, without an adequate seal, it is possible to leak fluid out of the access site in the gallbladder which could negatively impact the effectiveness of the aspiration, and potentially could also allow bile to seep out through the access site (around the shaft) during infusion. The potential issues are avoided by the inclusion and use of the inflatable, sealing and anchoring balloon 560 during the various infusion and aspiration procedures described herein.

As mentioned above, fluid is infused using the one or more peripheral lumens 550, arranged between the inner and outer walls 564, 562 of the tubular shaft 505, and the infusion ports 535 direct stream(s) of fluid radially outward from the access sheath tip 530A 530F. One way of fabricating the peripheral lumens 550 and inflation lumen 565, such as illustrated in FIG. 61, is to utilize a small tube between the first, outer shaft wall 562 and the second, inner shaft wall 564 to form the inflation lumen 565, with the balance of the space between the first, outer shaft wall 562 and the second, inner shaft wall 564 forming the peripheral lumen 550. Another way of fabricating the peripheral lumens 550 can be achieved by creating grooves on an outer surface of a hollow cylinder, which forms the inner wall 564. These grooves can then be covered with another cylindrical, hollow shaft forming the outer wall 562 (rigid, flexible, heat shrink; FEP or PET, etc.) to cover the grooves and create the peripheral lumens 550 in between the inner and outer walls 564, 562.

Referring to FIG. 72, these outward, radial stream(s) of fluid (504) from the access sheath tip 530A-530E can be one, two, three, or as many as needed, streams of fluid, or it can also be one continuous radial stream. These outward stream(s) (504) act to create circular flow inside of the gallbladder (590, illustrated in FIG. 72). This turbulent flow stirs up the gallstones and prevents them from resting in the gallbladder in a location that is too far away from the access sheath tip 530A-530E. By mobilizing the gallstones with this radial fluid flow, it makes them much more amenable to immediate aspiration as the flow path (506) brings the gallstones towards the access sheath tip 530A-530E. This flow path can be continuous and circular or it can be intermittent and circular, or it can be randomized to simply just move the gallstone(s) or gallstone fragments and eventually they will cross paths with the aspiration fluid path 506 and be swept into the aspiration channel 574. Due to the novel design of the system 300F, a single syringe as an aspiration device 250, 250A, 250B, 250C, 250D can be used to facilitate cyclical irrigation and aspiration to successfully move the gallstone(s) or gallstone fragments out of the gallbladder and into the in-line filter chamber 578. Simply advancing and retracting the syringe plunger 265 in rapid succession will remove the gallstone(s) or gallstone fragments. Typically, about equal amounts of fluid (e.g., saline or another biologically inert fluid) are infused and then aspirated, e.g., 60 ml of saline are infused and 60 ml of fluid (with any gallstone(s) and/or gallstone fragments) are subsequently or concomitantly aspirated, for example and without limitation. Alternatively, not separately illustrated, two rigid hollow shafts may be utilized to provide such infusion/irrigation and aspiration.

In another embodiment, the aspiration device 250, 250A, 250B, 250C, 250D can be replaced with an electric pump 595 (illustrated in FIG. 72). One potential concern with using an aspiration device 250, 250A, 250B, 250C, 250D that is manually controlled by a user may be variability in how quickly or forcefully the user retracts and expels the plunger 265 of the aspiration device 250, 250A, 250B, 250C, 250, which may result in some amount or level of aspiration/infusion flow rate changes. A pump 595 can be implemented to be simple for the user, with user-independent settings for consistent results.

Referring to FIGS. 70, 71 and 73, in other embodiments, the single aspiration device 250, 250A, 250B, 250C, 250D of the system 300F can be replaced with a dual aspiration and infusion assembly 650, 675 of system 300G having two aspiration devices, a first aspiration device 250₁, 250A₁, 250B₁, 250C₁, or 250D₁ (250₁-250D₁) and a second aspiration device 250₂, 250A₂, 250B₂, 250C₂, or 250D₂ (250₂-250D₂). FIG. 70 illustrates a representative system 300G comprising an access sheath 600 and a first embodiment of a dual aspiration and infusion assembly 650. FIG. 71 illustrates a representative proximal hub 610 of the access sheath 600 of the system 300G. FIG. 73 illustrates a representative second embodiment of a dual aspiration and infusion assembly 675 which may be utilized as part of the representative eighth system 300G embodiment in place of the dual aspiration and infusion assembly 650. FIGS. 74-78 illustrate the operation of the dual aspiration and infusion assembly 650 and the rack and pinion gearing assembly 630 utilized to synchronize the operation of the first aspiration device 250₁-250D₁ and the second aspiration device 250₂-250D₂.

The advantage of using a dual aspiration and infusion assembly 650, 675 is that the infusion (irrigation) and aspiration can either be performed synchronously or cyclically to increase the efficiency of gallstone and/or gallstone fragment removal, such that the first aspiration device 250₁-250D₁ is infusing while the other, second aspiration device 250₂-250D₂ is simultaneously or concurrently aspirating. This can be accomplished using various mechanisms to mechanically link two syringe plungers 265, for example, such that their motion can be in phase or out of phase by any selected degree. For example, FIGS. 73, 74, and 78 shows two aspiration devices 250, 250A, 250B, 250C, 250D being out of phase by 180 degrees, allowing the second aspiration device 250₂-250D₂ to aspirate and the other first aspiration device 250₁-250D₁ to infuse or irrigate simultaneously. Apart from providing the simultaneous or concurrent infusion and aspiration, and with the inclusion of some additional valves as described in greater detail below, the system 300G otherwise has the same structure and operation as the system 300F.

Referring to FIGS. 70-78, the system 300G for gallstone removal comprises several components, including an access sheath 600 and a dual aspiration and infusion assembly 650 (or a dual aspiration and infusion assembly 675). More specifically, in a representative embodiment, a system 300G comprises an access sheath 600 coupled through a proximal hub 610 to a dual aspiration and infusion assembly 650 or 675, such as illustrated in FIGS. 70, 71, and 73. As with the various systems described above, the system 300G may further include a catheter 400-400H, such as illustrated in FIG. 63, arranged through one or more access ports 130A. Apart from the differences described in greater detail below, the system 300G functions similarly to the other systems 300-300F described above. As with the system 300F, the notable differences of system 300G are the inclusion of multiple, separate infusion and aspiration channels 540, 574, respectively, the inclusion of a filter assembly 515 in the proximal hub 610 as part of the aspiration channel 574, and the optional inclusion of a sealing and anchoring balloon 560, which further utilizes a separate inflation lumen 565. Additional differences are described in greater detail below.

The access sheath 600 comprises a cylindrical, hollow, tubular shaft or member 505 coupled to a proximal hub 610. The tubular shaft 505 of the system 300G has the identical structure and function as previously described for the system 300F. As mentioned above, the proximal hub 610 includes additional valve, tubing, and coupling components, as described in greater detail below.

In a representative embodiment, the proximal hub 610 also comprises a housing 576 having a plurality of hub lumens 570, 575, 580, 585; a filter assembly 515 having a filter chamber 578; infusion tubing 545A, 545B; a plurality of valves 120B, 120C, 125A, 125B, 125C, 125D coupled within the housing 576 and the tubing 545A, 545B and return path tubing 625, and a plurality of mating couplings 150E and 155E, 150F and 155F, and couplings 150G and 150H. Mating couplings 150G and 155G removably couple the first aspiration device 250₁-250D₁ to the proximal hub 610. Mating couplings 150H and 155H removably couple the second aspiration device 250₂-250D₂ to the proximal hub 610.

Mating couplings 150E and 155E connect the infusion tubing 545A to the hub lumen 570, which in turn is in fluid communication with the peripheral lumens 550. Mating couplings 150F and 155F connect the tubular shaft 505 to the proximal hub 610. The valves 120B, 120C, 125A, 125B, 125C, and 125D are typically each one-way valves, such as stop cock valves or pressure cracking valves (illustrated in FIG. 71 as pressure cracking valves 610 for valves 120B, 120C), and may each be implemented as previously described. Also, the mating couplings 150E and 155E, 150F and 155F, 150G and 155G, and 150H and 155H, may each be implemented as previously described, such as quick connect fittings, screw threads, snap fittings, etc.

For the proximal hub 610, like the proximal hub 510, the hub lumen 570 is also arranged between the infusion tubing 545A and the peripheral lumens 550. The valve 120C is also arranged between the central lumen 555 of the tubular shaft 505 and the hub lumen 575. The hub lumen 575 is also arranged between the valve 120C and the filter 520, as part of the filter assembly 515. The filter chamber 578 is also the interior portion of the filter 520, and is in fluid communication with the hub lumen 575, forming the space within the filter 520 for filtering out and trapping any aspirated gallstone(s) or gallstone fragments. The filter canister lumen 580 is also within the filter canister 525, on the side of the filter 520 opposite the filter chamber 578. The one-way valve 125A separates the filter canister lumen 580 from the hub lumen 585, and serves to prevent injected infusion fluid from entering the filter canister lumen 580 and the balance of the aspiration channel 574.

The proximal hub 610 further includes return path tubing 625, having the one-way valve 125C, coupled between the hub lumen 585 and the infusion tubing 545B, with the infusion tubing 545B extending between the coupling 150G and the one-way valve 125B.

Referring again to FIGS. 58-63 and 70-71, those having skill in the art will recognize that the various tubing structures of the proximal hubs 510, 610, such as the infusion tubing 545, infusion tubing 545A, return path tubing 625, and the other various components forming the infusion channel 540 and the aspiration channel 574, may all be integrated into a single housing, not separately illustrated, and any and all such embodiments and variations are considered equivalent and within the scope of the disclosure. Such a singular housing has the additional advantage of eliminating any requirement for various connectors, such as mating couplings 150F and 155F and/or couplings 150E and 155E, for example and without limitation. Those having skill in the art will also recognize that the tubular shaft 505 may also be integrated with the proximal hubs 510, 610, and any and all such embodiments and variations are considered equivalent and within the scope of the disclosure. Those having skill in the art will also recognize that the various tubings, tubular shafts, and proximal hubs may have a wide variety of shapes, sizes, and configurations, and any and all such embodiments and variations are considered equivalent and within the scope of the disclosure.

In operation of the system 300G, fluid is typically infused into the subject gallbladder to generate some turbulence to stir up any gallstone(s) or gallstone fragments for aspiration, as described in greater detail below. For infusion of fluid in the infusion channel 540, fluid from the first aspiration device 250₁-250D₁ is injected through infusion tubing 545B (using a plunger 265 of the aspiration device 250, 250A, 250B, 250C, 250D) and through one-way valve 125B (and valve 125D) and into the infusion tubing 545A. In turn, the fluid from the infusion tubing 545A flows through valve 120B (e.g., a pressure cracking valve 610) and into the hub lumen 570, which is in fluid communication with each of the peripheral lumens 550. The infusing fluid then flows through each of the peripheral lumens 550 and radially outward through each of the corresponding infusion ports 535, as mentioned above.

The filter assembly 515 of the proximal hub 610 also forms part of the aspiration channel 574, and has the structure and function described above for proximal hub 510. Aspiration of fluid, gallstone(s) or gallstone fragments differs using proximal hub 610 insofar as the filtered fluid (and particles not captured in the filter 520) is aspirated through the valve 125A and hub lumen 585 and into the second aspiration device 250₂-250D₂. Any aspirated gallstone(s) or gallstone fragments also remain in the filter chamber 578 and do not pass through the filter 520, and further are prevented from exiting back into the central lumen 555 by the valve 120C. Any remaining fluid flows through the filter 520 and into the filter canister lumen 580, then through the (one-way) valve 125A into the hub lumen 585 and into the second aspiration device 250₂-250D₂, in a first infusion and aspiration phase. As described in greater detail below, using the proximal hub 610, in a second infusion and aspiration phase, the fluid is returned to the first aspiration device 250₁-250D₁, and another infusion and aspiration sequence may be repeated.

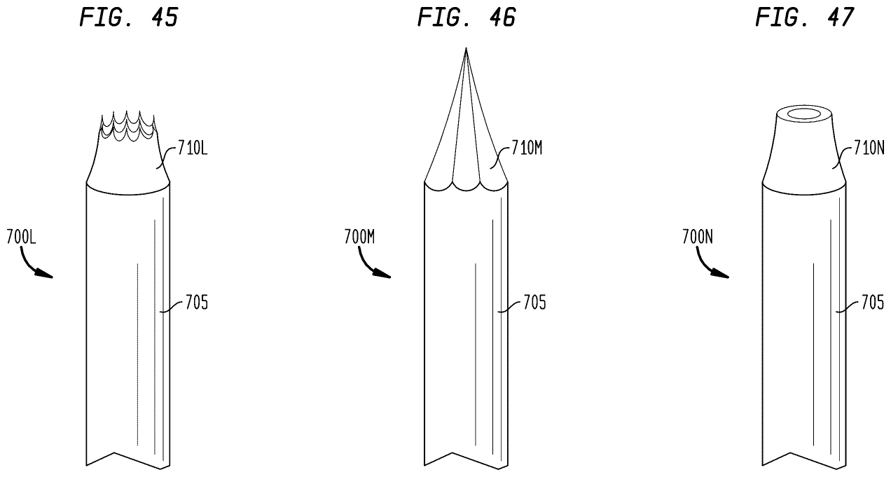

It should be noted that an additional valve 125D is provided as an option in the infusion tubing 545A of the proximal hub 610, illustrated in FIG. 45 as a stopcock valve. During infusion, the stopcock valve 125D may be turned on and off, allowing and ceasing fluid flow, respectively, which may serve to advantageously increase turbulence in and disrupt the fluid flow within the subject gallbladder.

The control and operation of the first aspiration device 250₁-250D₁ and second aspiration device 250₂-250D₂ may be performed manually or mechanically, such as through implementation and use of the dual aspiration and infusion assembly 650 (or 675).

As illustrated in FIG. 73, a pump 680 having the illustrated linkage assembly 685 may be implemented to operate the first aspiration device 250₁-250D₁ and second aspiration device 250₂-250D₂ out of phase, such as 180 degrees out of phase. As illustrated in FIGS. 79, 71, and 74-78, a rack and pinion gearing assembly 630 may be utilized, with a first rack gear 635 coupled to a plunger 265 of the first aspiration device 250₁-250D₁, a second rack gear 640 coupled to the second aspiration device 250₂-250D₂, with the first and second rack gears 635, 640 engaging with a pinion gear 645, and coupled and held in place to the first aspiration device 250₁-250D₁ and to the second aspiration device 250₂-250D₂ using a clamping assembly 655, for example and without limitation. Retracting one syringe plunger 265 coupled to the first rack gear 635 would rotate the pinion gear 645 which would be coupled to the second rack gear 640 coupled to the second syringe plunger 265, causing it to advance, and vice-versa.

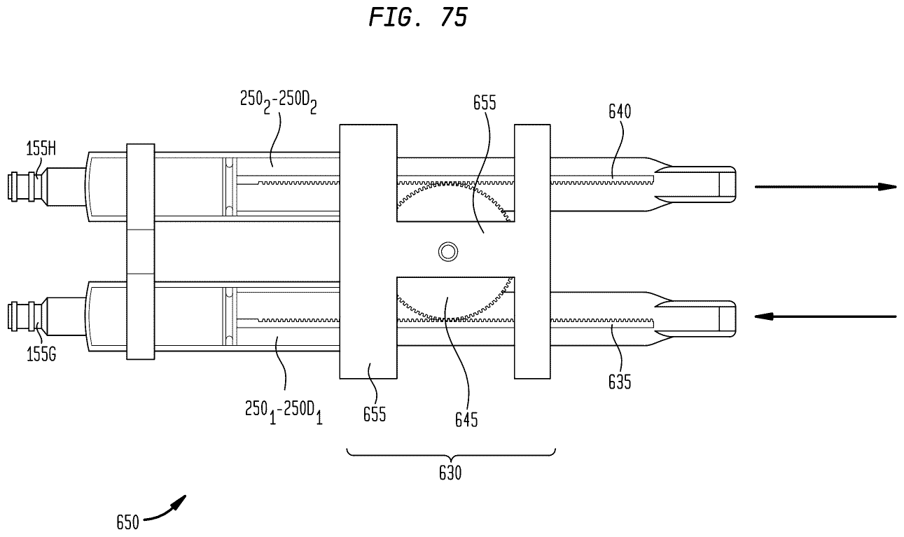

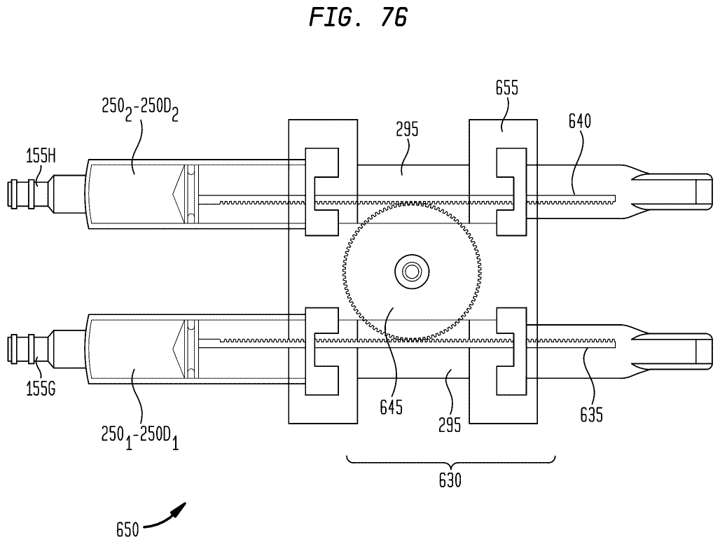

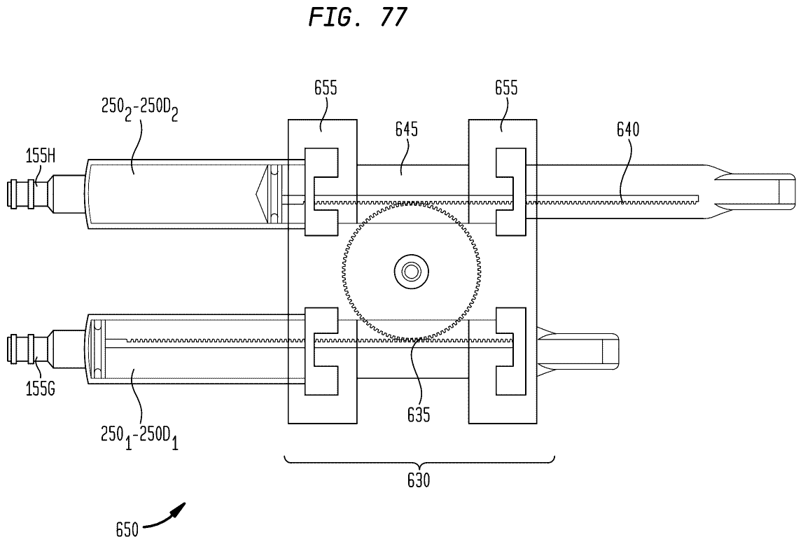

FIGS. 74-78 illustrate the structure and operation of the dual aspiration and infusion assembly 650 having the rack and pinion gearing assembly 630. The most notable difference in using the dual aspiration and infusion assembly 650 or 675 is that, during a first infusion and aspiration phase, starting with fluid (such as saline) within the first aspiration device 250₁-250D₁ and an empty second aspiration device 250₂-250D₂ (FIG. 74), while the first aspiration device 250₁-250D₁ is infusing fluid (advancing its syringe plunger 265) through the peripheral lumens 550 of the tubular shaft 505 of the access sheath 600 and into the subject gallbladder, the second aspiration device 250₂-250D₂ is simultaneously aspirating fluid (retracting its syringe plunger 265) through the central lumen 555 of the tubular shaft 505, through the filter assembly 515, through valve 125A, hub lumen 585, and into the second aspiration device 250₂-250D₂ (FIGS. 75 and 76). At the end of this first infusion and aspiration phase, the first aspiration device 250₁-250D₁ is now empty and the infused fluid has been aspirated into the second aspiration device 250₂-250D₂, which is now effectively full (FIG. 77).

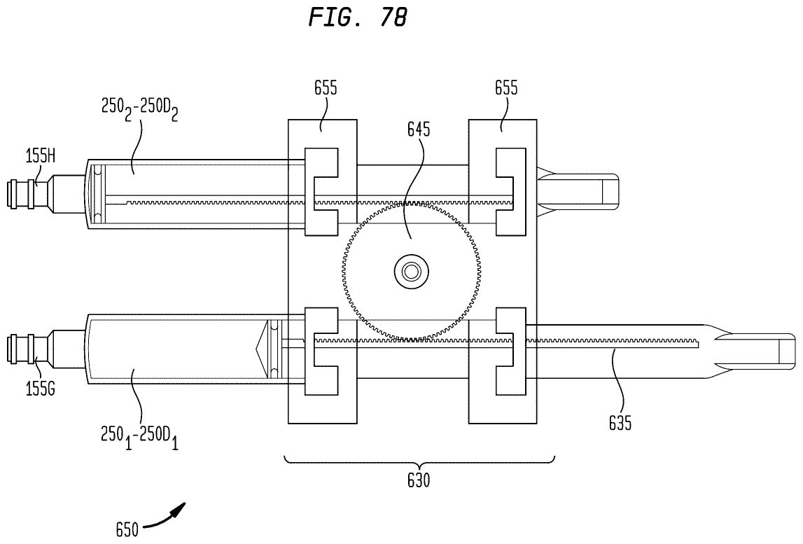

In a second infusion and aspiration phase, the filtered, aspirated fluid held by the second aspiration device 250₂-250D₂ is returned to the currently empty first aspiration device 250₁-250D₁. The filtered, aspirated fluid is injected by the second aspiration device 250₂-250D₂ (advancing its syringe plunger 265) into the hub lumen 585, and flow of this filtered, aspirated fluid is directed using the one-way valve 125C in the return path tubing 625 into infusion tubing 545B. While the filtered, aspirated fluid is being injected by the second aspiration device 250₂-250D₂, the filtered, aspirated fluid is simultaneously aspirated (retracting its syringe plunger 265) by the first aspiration device 250₁-250D₁, with the pressure differential caused by withdrawing the plunger 265 in the first aspiration device 250₁-250D₁ causing the fluid to flow into the first aspiration device 250₁-250D₁ rather than through the valve 125B. At the end of this second infusion and aspiration phase, the second aspiration device 250₂-250D₂ is now empty and the fluid has been aspirated into the first aspiration device 250₁-250D₁ (FIG. 78), which is now effectively full, and a next first infusion and aspiration phase may begin.

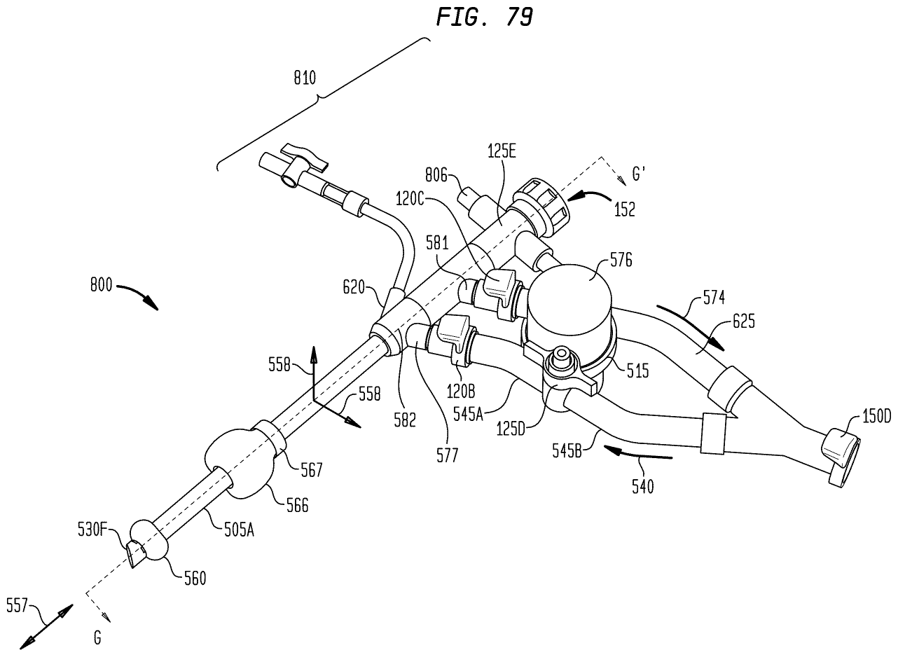

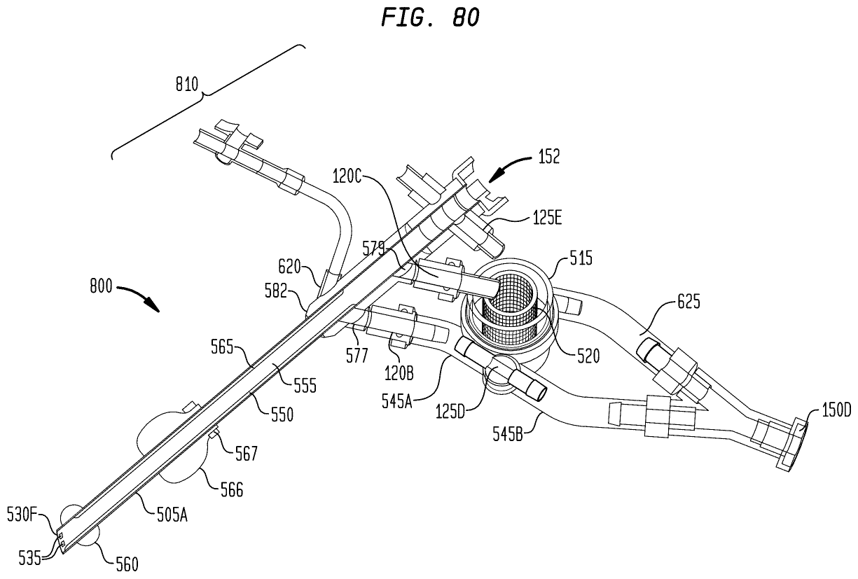

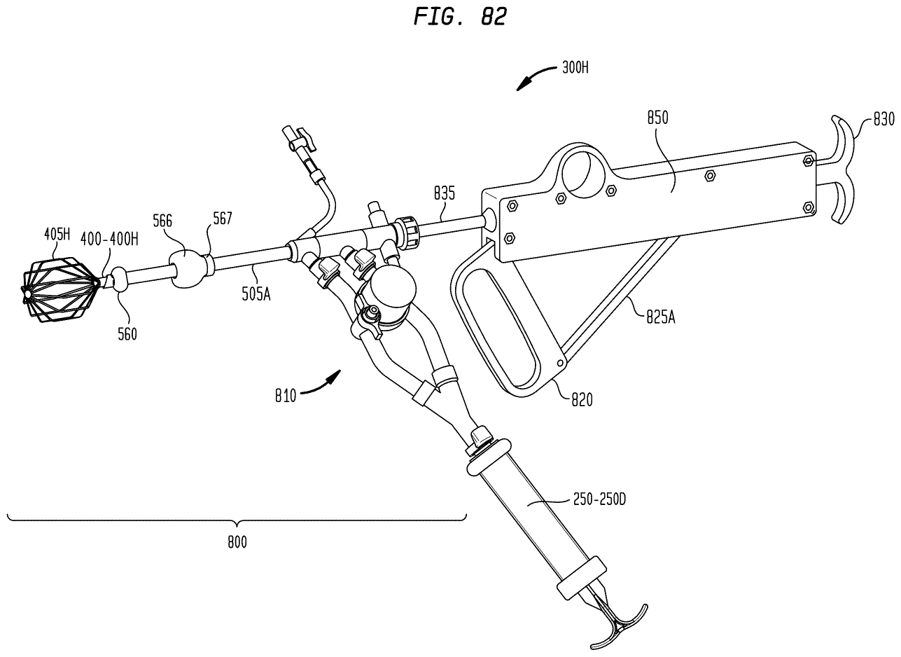

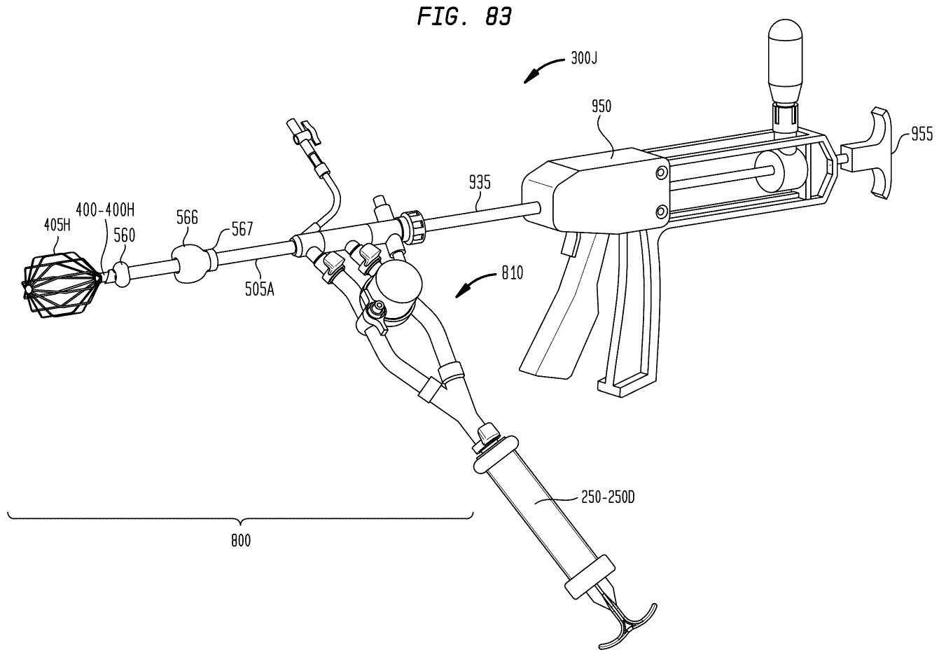

FIG. 79 is an isometric view illustrating a representative eighth embodiment of an access sheath 800. FIG. 80 is a cut-away view illustrating the representative eighth embodiment of an access sheath 800 of FIG. 79. FIG. 81 is a partial cross-sectional view (through the G-G′ plane of FIG. 79) illustrating the representative eighth embodiment of the access sheath 800, additionally with a representative catheter embodiment. FIG. 82 is an isometric view illustrating a representative ninth embodiment of a system 300H having the access sheath 800 and a first embodiment of a crushing or fracturing control apparatus 850. FIG. 83 is an isometric view illustrating a representative tenth embodiment of a system 300J having the access sheath 800 and a second embodiment of a crushing or fracturing control apparatus 950.

Referring to FIGS. 79-83, the access sheath 800 comprises a proximal hub 810 having a housing 582 and coupled at a first end to a tubular shaft 505A (or 505), and also coupled at a second end to a valve 125E. The proximal hub 810 includes a filter assembly 515 as part of the aspiration channel 574 and also includes an infusion channel 540, as previously described. As in the other access sheaths 500, 500A, 600, 600A, the access sheath 800 is also coupleable (through connector 150D) to aspiration device 250, 250A, 250B, 250C, 250D, in the single configuration (as illustrated) or in the dual configuration described above. The proximal hub 810 also includes a first port 577 in fluid communication with the peripheral lumens 550 and coupled through valve 120B to infusion tubing 545A, 545B; a second port 581 in fluid communication with the hub lumen 575 of the proximal hub 810 and coupled through valve 120C (or return path tubing 625) to the filter assembly 515; and a third port 620 in fluid communication with the inflation lumen 555.

The access sheath 800 differs from the access sheaths 500, 500A, 600, 600A in several ways. First, the filter assembly 515 is no longer in-line (linearly aligned) with the tubular shaft 505A, and instead is off axis, i.e., spaced-apart or offset transversely, from the central lumen 555. Stated another way, the infusion and aspiration channels 540, 574 are not linearly aligned (in-line) with the tubular shaft 505A and access the tubular shaft 505A through the proximal hub 810, with the infusion channel 540 coupled to the hub lumen 575 of the proximal hub 810 for fluid communication with the peripheral lumen(s) 550, and with the aspiration channel 574 coupled to the hub lumen 575 of the proximal hub 810 for fluid communication and aspiration through the central lumen 555 of the tubular shaft 505A.

This provides for a straight insertion of a catheter 400-400H in-line through the valve 125E, into the proximal hub 810, and into the central lumen 555 of the tubular shaft 505, 505A, such as illustrated in FIG. 81, i.e., without any bending of the catheter 400-400H which would otherwise be required if the catheter 400-400H were inserted through an access port 130, 130A, 135. This off-line configuration of the filter assembly 515, with the in-line configuration of the valve 125E, into the proximal hub 810, and into the central lumen 555 of the tubular shaft 505, 505A, also allows such a catheter 400-400H to have a rigid or stiff configuration. Furthermore, this off-line configuration of the filter assembly 515, with the in-line configuration of the valve 125E, into the proximal hub 810, and into the central lumen 555 of the tubular shaft 505, 505A, also allows in-line use of the crushing or fracturing control apparatus 850, 950, which may be inserted (typically with a catheter 400F-400H and a crushing or fracturing instrument 700-700S), straight and in-line through the valve 125E, into the proximal hub 810, and into the central lumen 555 of the tubular shaft 505, 505A, such as illustrated in FIGS. 82 and 83, also without any bending. In a representative embodiment, the valve 125E is implemented as a hemostasis valve, such as available from Inari Medical, Inc., having a button actuator 806 which opens a passage within the valve 125 for insertion of a crushing or fracturing control apparatus 850, 950, catheter 400F-400H, and/or a crushing or fracturing instrument 700-700S.

Second, the access sheath 800 also includes an adjustable stopper 566 (having an adjustment ring 567) between the proximal hub 810 and the sealing and anchoring balloon 560. The adjustable stopper 566 is slideable along the tubular shaft 505, 505A and may be secured at any selected location along the tubular shaft 505, 505A by tightening the adjustment ring 567. In use, when the access sheath 800 is inserted into a patient or subject gallbladder, sealing and anchoring balloon 560 is inflated and the adjustable stopper 566 is slid and secured to abut the skin of the patient or subject, so that the access sheath 800 is stabilized and does not move or insert further into the patient or subject.

Apart from these differences, the access sheath 800 functions as previously described for the access sheaths 500, 500A, 600, 600A, with infusion provided through the infusion channel 540 and aspiration provided through the aspiration channel 574 having the filter assembly 515. Those having skill in the field will also recognize that the access sheath 800 may also be configured to use a dual aspiration and infusion assembly 650, 675 as described above, not separately illustrated.

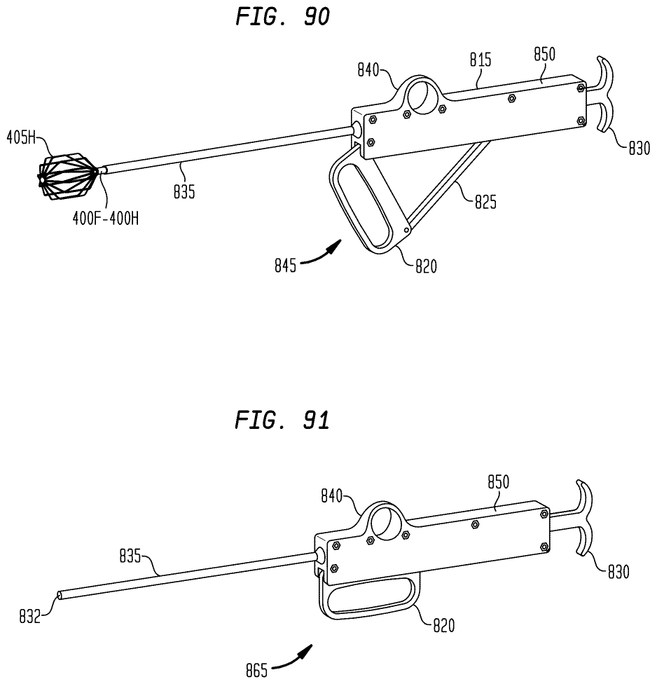

Referring to FIGS. 82 and 83, a system 300H comprises an access sheath 800, a catheter 400F-400H arranged within a crushing or fracturing control apparatus 850, the crushing or fracturing control apparatus 850 (having a crushing or fracturing shaft 835 and the catheter 400F-400H) coupled or inserted through the proximal hub 810 and into the tubular shaft 505A of the access sheath 800, and an aspiration device 250, 250A, 250B, 250C, 250D coupled to the access sheath 800 for infusion and aspiration. Referring to FIG. 83, a system 300J comprises an access sheath 800, a catheter 400F-400H arranged within a crushing or fracturing control apparatus 950, a crushing or fracturing control apparatus 950 (having a crushing or fracturing shaft 935 and the catheter 400F-400H) coupled or inserted through the proximal hub 810 into the tubular shaft 505A of the access sheath 800, and an aspiration device 250, 250A, 250B, 250C, 250D coupled to the access sheath 800 for infusion and aspiration. FIGS. 82 and 83 also illustrate the systems 300H and 300J with a catheter 400F 400H having a cage or basket 405H (as an example of a basket or cage 405-405K) extending from the tubular shaft 505A, which as described in greater detail below with reference to FIGS. 90-97, is controlled using a crushing or fracturing control apparatus 850, 950 arranged as illustrated for the systems 300H and 300J.

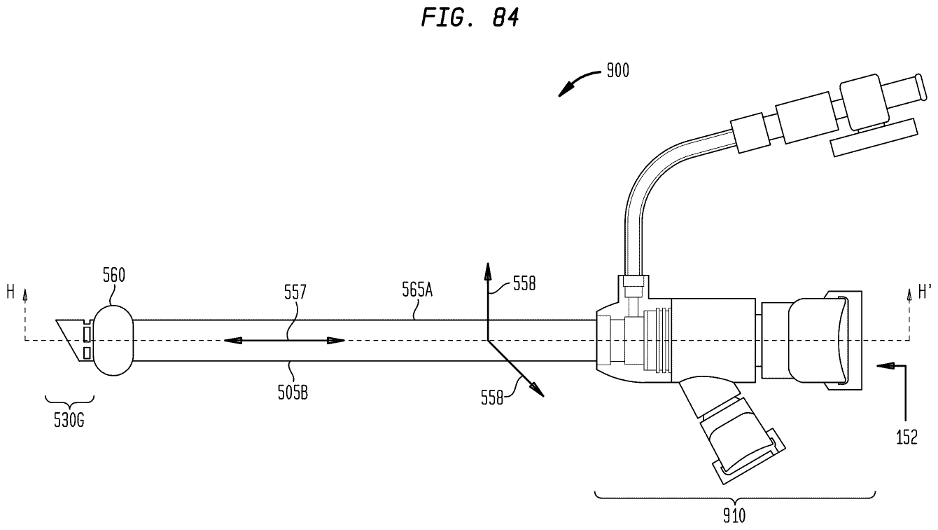

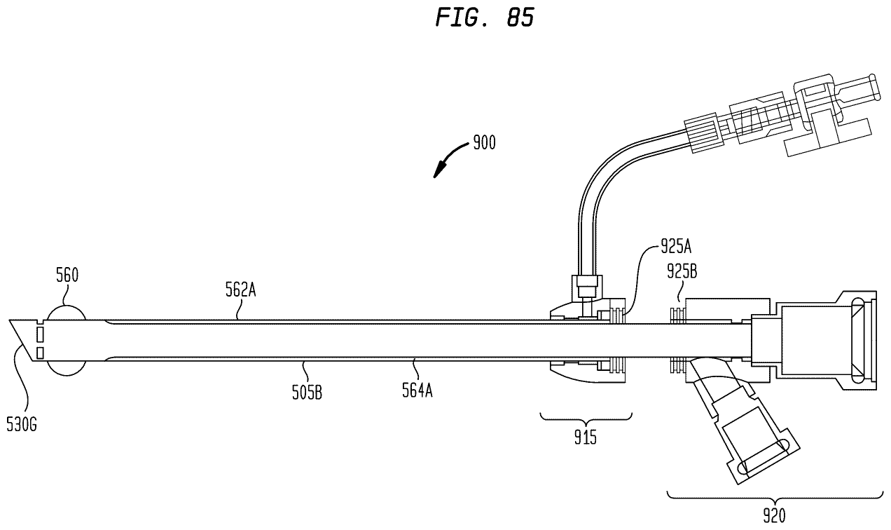

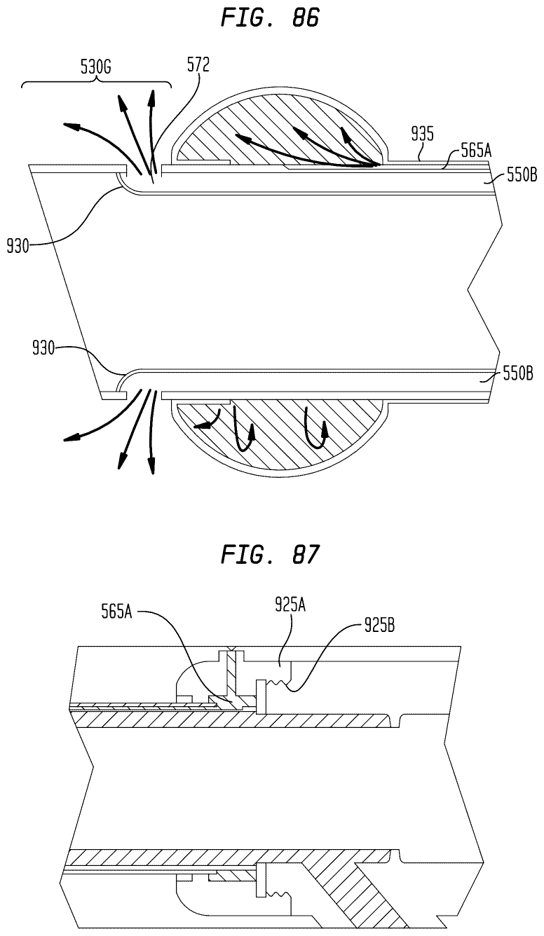

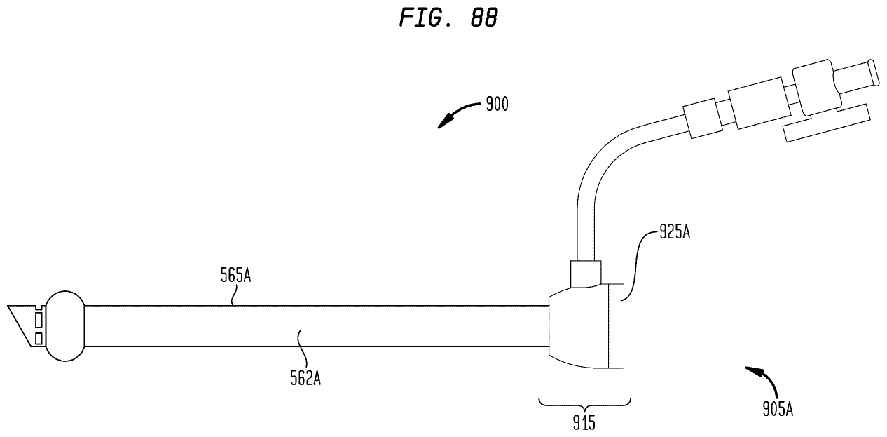

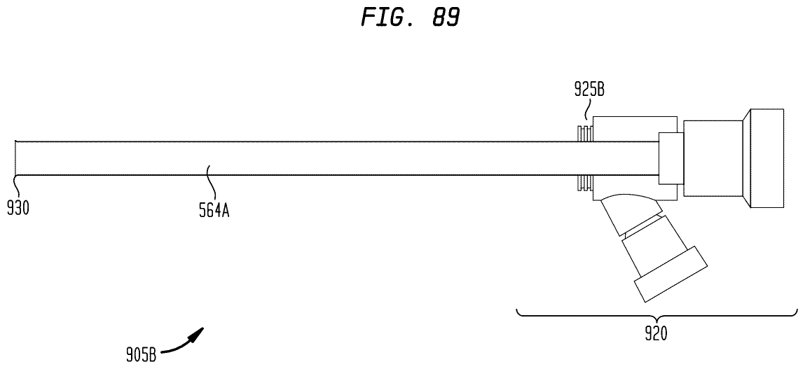

FIG. 84 is an isometric view illustrating a representative two-part ninth embodiment of an access sheath 900. FIG. 85 is a cut-away view illustrating the representative two-part ninth embodiment of an access sheath 900. FIG. 86 is a first cross-sectional view (through the H-H′ plane of FIG. 84) illustrating a seventh embodiment of an access sheath tip of the representative ninth embodiment of an access sheath 900. FIG. 87 is a second cross-sectional view (through the H-H′ plane of FIG. 84) illustrating a portion of a proximal hub of the representative ninth embodiment of an access sheath 900. FIG. 88 is an isometric view illustrating a first removably coupleable part 905A of the representative ninth embodiment of an access sheath 900. FIG. 89 is an isometric view illustrating a second removably coupleable part 905B of the representative ninth embodiment of an access sheath 900.

Referring to FIGS. 84-89, the access sheath 900 comprises a proximal hub 910 coupled to a tubular shaft 505B. The access sheath 900 is separable into two parts, a first removably coupleable part 905A and a second removably coupleable part 905B. The tubular shaft 505B is also removably separable into two component parts, the first, outer shaft wall 562A and the second, inner shaft wall 564A. The proximal hub 910 is separable into two parts, a first proximal hub section 915 (coupled to the first, outer shaft wall 562A) and a second proximal hub section 920 (coupled to the second, inner shaft wall 564A), using corresponding, mating screw thread connectors 925A, 925B respectively. The first proximal hub section 915 coupled to the first, outer shaft wall 562A forms the first removably coupleable part 905A of the access sheath 900 and the second proximal hub section 920 coupled to the second, inner shaft wall 564A forms the second removably coupleable part 905B of the access sheath 900. While not separately illustrated, any of the other various access sheaths 100, 100A, 200, 200A, 500, 500A, 600, 600A, 800 may also be modified to have this or a similar two-part configuration.

For this access sheath 900 embodiment, the tubular shaft 505B has a different configuration, with an inflation lumen 565A arranged as an outer groove on the exterior of the first, outer shaft wall 562A and is covered with a flexible material (such as utilized to form the sealing and anchoring balloon 560), as illustrated in FIG. 86. The inflation lumen 565A extends into the first proximal hub section 915 as illustrated in FIG. 87 to receive fluid or air, for example, to inflate the sealing and anchoring balloon 560. In addition, the second, inner shaft wall 564A of the tubular shaft 505B has a curvature (or flair) 930 at the distal end of the second, inner shaft wall 564A to fit snugly against the inner surface of the first, outer shaft wall 562A, as illustrated in FIG. 86, with the infusion lumen 550B provided by the space in between the first, outer shaft wall 562A and the second, inner shaft wall 564A, as described above. Those having skill in the field will also recognize that the access sheath 900 may also be configured to use any aspiration device 250, 250A, 250B, 250C, 250D, a dual aspiration and infusion assembly 650, 675, an infusion channel 540, and/or an aspiration channel 574 having the filter assembly 515, as described above, not separately illustrated.

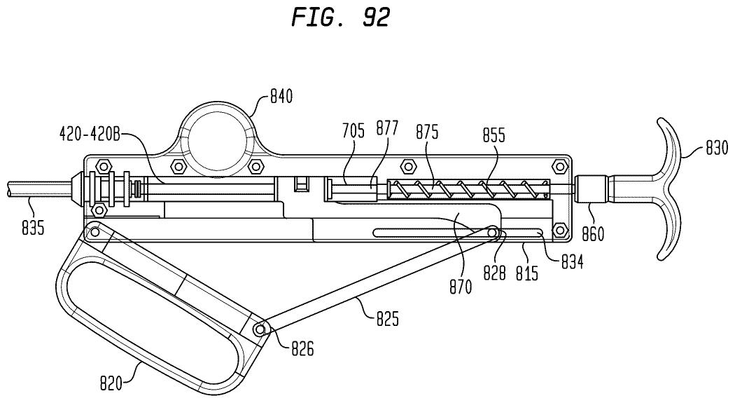

FIG. 90 is a first isometric view illustrating a representative first embodiment of a crushing or fracturing control apparatus 850 with a representative catheter 400F-400H embodiment, illustrated with a cage or basket 405H. FIG. 91 is a second isometric view illustrating the representative first embodiment of a crushing or fracturing control apparatus 850. FIG. 92 is a partial cut-away view illustrating the representative first embodiment of a crushing or fracturing control apparatus 850 with a representative catheter 400F-400H embodiment of FIG. 90. The crushing or fracturing control apparatus 850 comprises a housing 815, a control apparatus shaft 835 coupled to the housing 815, a catheter control assembly 892, a first handle 820, a control linkage lever 825, a crushing or fracturing spring 855, and a second handle 830. The catheter control assembly 892 comprises a catheter control actuator 870 and a crushing or fracturing pin 875. The control apparatus shaft 835 has a lumen 832 for insertion of a catheter 400F-400H and a crushing or fracturing instrument 700-700S arranged coaxially within the inner catheter lumen 435 of the inner catheter manipulation shaft 420, 420A of the catheter 400F-400H.

The first handle 820 is pivotably coupled to the housing 815 and is also moveably (pivotably) coupled to a first end 826 of the control linkage lever 825. The catheter control actuator 870 is moveably (pivotably) coupled to a second end 828 of the control linkage lever 825, and both the catheter control actuator 870 and the control linkage lever 825 are slidable in a slot 834 of the housing 815. The catheter control actuator 870 includes a tubular catheter grip 894 linearly aligned with the control apparatus shaft lumen 832, and the tubular catheter grip 894 is coupleable around the inner catheter manipulation shaft 420, 420A of the catheter 400F-400H to secure and move the inner catheter manipulation shaft 420, 420A longitudinally within the catheter 400F-400H (and lumen 832 of the control apparatus shaft 835) in response to movement of the first handle 820 and the control linkage lever 825. The crushing or fracturing pin is linearly aligned with (coaxial within) the tubular catheter grip 894 within the housing 815 to engage with the crushing or fracturing instrument 700 700S, with the crushing or fracturing instrument 700-700S arranged coaxially within the inner catheter lumen 435 of the inner catheter manipulation shaft 420, 420A of the catheter 400F-400H. The second handle 830 (with a removable retention clip 860) is coupled to the crushing or fracturing pin 875, i.e., arranged to control the crushing or fracturing coil spring 855 and move the crushing or fracturing pin 875. The control apparatus shaft 835 is also insertable into an access sheath 800, for example and without limitation, such as illustrated in FIG. 82. The housing 815 may also include an optional thumb grip 840 to stabilize the user's hand. The crushing or fracturing control apparatus 850 is typically utilized and operated by medical personnel, holding the housing 815 and using the first and second handles 820, 830 and optional thumb grip 840. The various components of the crushing or fracturing control apparatus 850 may be comprised of a wide variety of materials, such as stainless steel, titanium, a medical/surgical grade polymer, and so on, for example and without limitation.

In operation, a catheter 400F-400H, with a crushing or fracturing instrument 700-700S arranged coaxially in the inner catheter lumen 435 of the catheter 400F-400H, is inserted into the lumen 832 of the control apparatus shaft 835. The inner catheter manipulation shaft (or member) 420, 420A of the catheter 400F-400H is arranged to engage with the catheter control actuator 870, and the crushing or fracturing instrument 700-700S is arranged to engage with the crushing or fracturing pin 875. With the first handle 820 in a first position 865 (illustrated in FIG. 91), the cage or basket 405G, 405H, 405J, 405K of the catheter 400F-400H is held within the outer catheter tube or member 415B, 415C of the catheter 400F-400H. In this arrangement, the crushing or fracturing instrument 700-700S is arranged coaxially in the inner catheter lumen 435 of the inner catheter manipulation shaft 420B within the catheter 400F-400H, which in turn is arranged coaxially within a lumen of the control apparatus shaft 835. The control apparatus shaft 835 (having the catheter 400F 400H and crushing or fracturing instrument 700-700S) is then inserted into an access sheath 800 arranged in a patient's or subject's gallbladder, for example and without limitation.

With the first handle 820 then moved into a second position 845 (illustrated in FIG. 90) to move the control linkage lever 825, the catheter control actuator 870 engages with and pushes the inner catheter manipulation shaft 420B, advancing the cage or basket 405G, 405H, 405J, 405K out of the inner catheter lumen 435 of the inner catheter manipulation shaft 420, 420A of the catheter 400F-400H and out of the crushing or fracturing shaft 835, as illustrated in FIG. 90. The expanded cage or basket 405G, 405H, 405J, 405K may then be utilized to capture a gallstone 411, as previously described. When a gallstone is then within the interior of the cage or basket 405G, 405H, 405J, 405K, the first handle 820 is then moved toward the first position 865, which retracts or pulls the inner catheter manipulation shaft 420B back into the outer catheter tube or member 415B, 415C (and control apparatus shaft 835), thereby retracting the struts 410 and contracting the cage or basket 405G, 405H, 405J, 405K around the gallstone. With the cage or basket 405G, 405H, 405J, 405K around the gallstone, the first handle 820 is typically in a position intermediate or between the first and second positions 865, 845. Depending on the size of the captured gallstone, the captured gallstone may then be removed from the patient or subject by withdrawing the control apparatus shaft 835 of the crushing or fracturing control apparatus 850 from the access sheath 800. For a gallstone larger than the inner diameter of the central lumen 555 of the access sheath 800, the captured gallstone may be crushed or fractured using one of the crushing or fracturing instruments 700-700S within the inner catheter shaft lumen 435.

In the event the gallstone is to be crushed or fractured, while the cage or basket 405G, 405H, 405J, 405K is contracted around and securing the gallstone, a crushing or fracturing instrument 700-700S may be advanced through the inner catheter shaft lumen 435 (such as illustrated in FIG. 54) to impact the gallstone, once or repeatedly, as needed to crush or fracture the gallstone, using the second handle 830 and force supplied by the crushing or fracturing coil spring 855 through crushing or fracturing pin 875, which engages with the proximal end 877 of the shaft 705 of the crushing or fracturing instrument 700-700S. In a first step, the second handle 830 is or has been pulled back (away from the housing 815) to compress the crushing or fracturing coil spring 855, and the second handle 830 is locked in the compressed spring position using a removable retention clip 860. To impact the gallstone with a crushing or fracturing instrument 700-700S, in a second step, the removable retention clip 860 is removed, releasing the second handle 830 and the compressed crushing or fracturing coil spring 855, which advances (moves distally) the crushing or fracturing pin 875, which in turn advances (moves distally) the crushing or fracturing instrument 700-700S, driving the crushing or fracturing tip 710-710S into and thereby crushing or fracturing the gallstone, typically using an impulse force of 15-20 lbf (66.7-90 N), for example and without limitation. These first and second steps may be repeated as may be necessary or advisable.

As another option, not separately illustrated, the crushing or fracturing control apparatus 850 may include a release button (in the housing 815) for the second handle 830. The second handle 830 may be pulled and locked into an extended position, storing energy in the crushing or fracturing coil spring 855. When actuated using such a release button, the crushing or fracturing coil spring 855 is released and the crushing or fracturing instrument 700-700S is advanced to impact a gallstone.

The fractured or crushed gallstone pieces may be small enough to exit from the cage or basket 405G, 405H, 405J, 405K and be aspirated (or removed using the cage or basket 405G, 405H, 405J, 405K), as described above. As the gallstone is crushed or fractured, feedback may also be provided to the medical personnel, as the cage or basket 405G, 405H, 405J, 405K may contract further around the fractured or crushed gallstone pieces, allowing the first handle 820 to be moved back closer to the first position 865.

Typically, the first crushing or fracturing control apparatus 850 is removed from the access sheath 800, and the gallstone fragments are aspirated, using the aspiration channel 574 having the filter assembly 515, as described above.

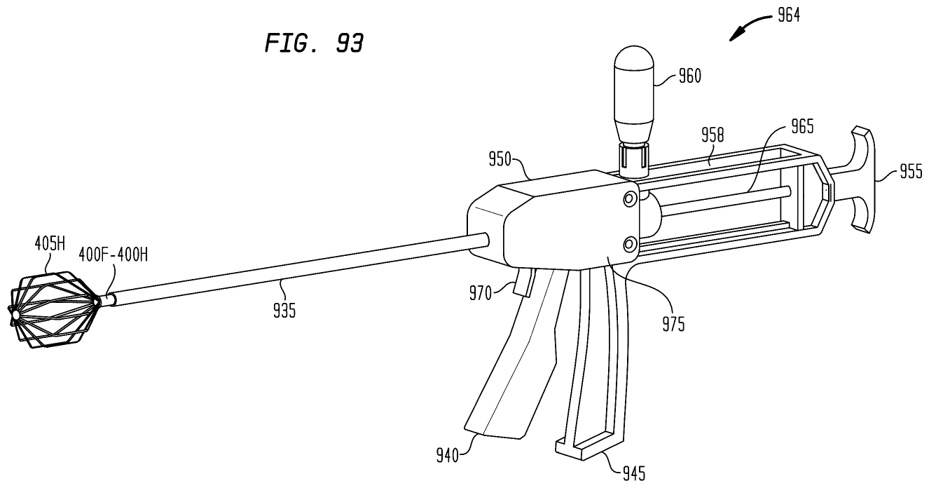

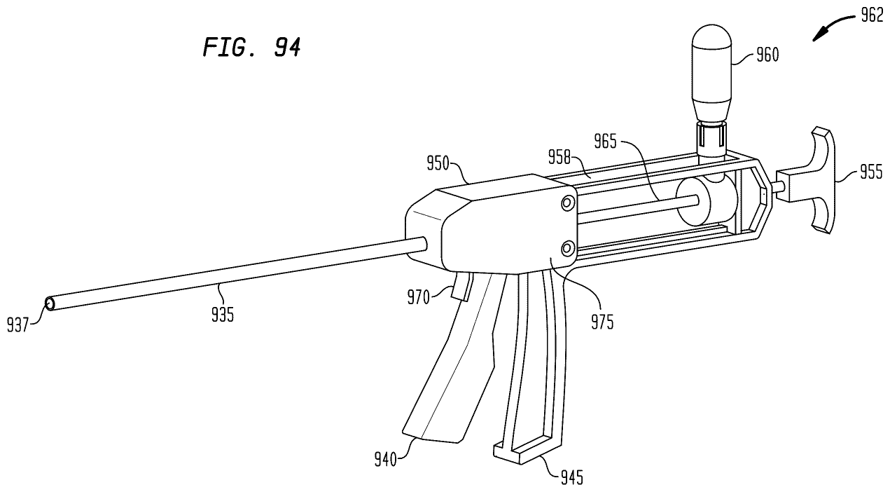

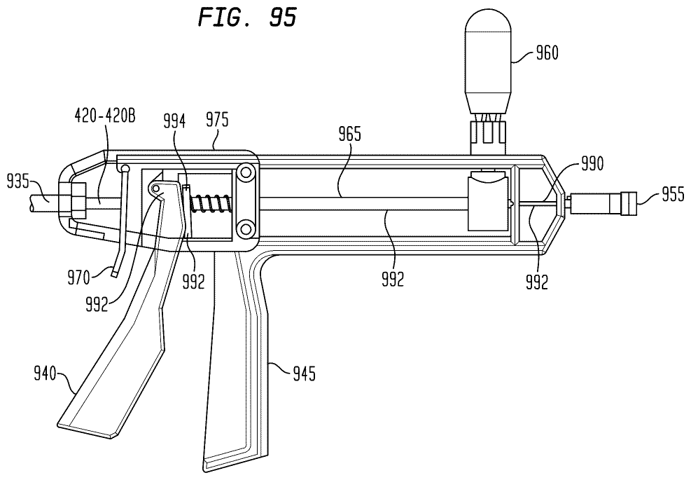

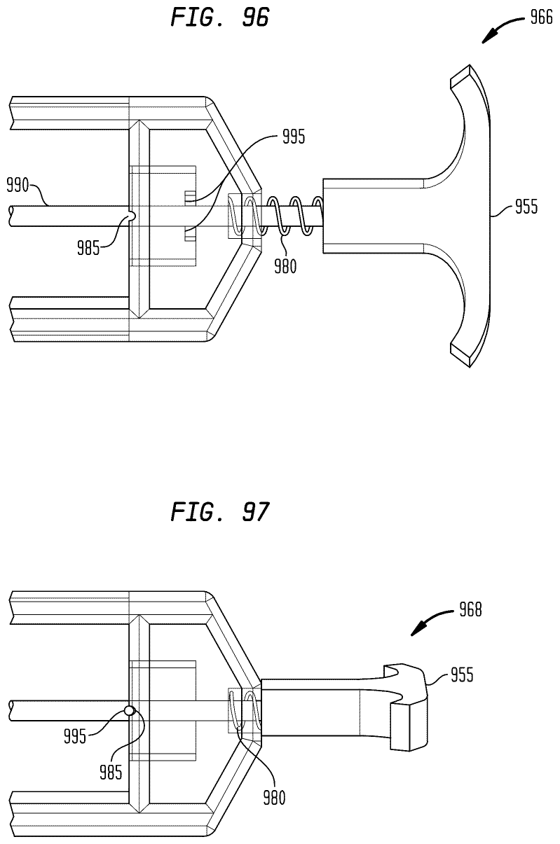

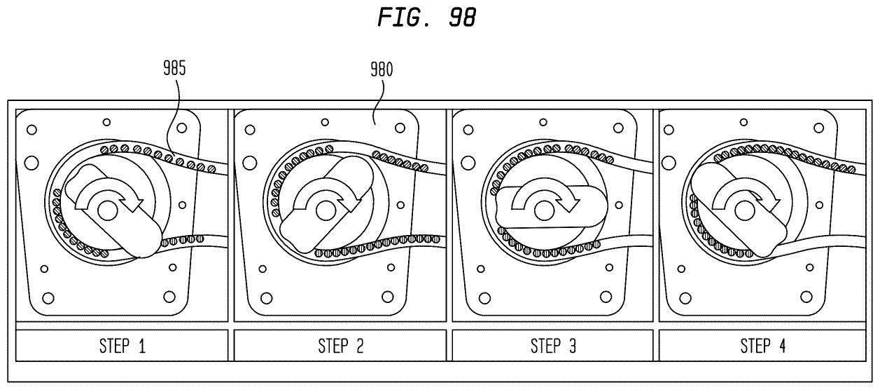

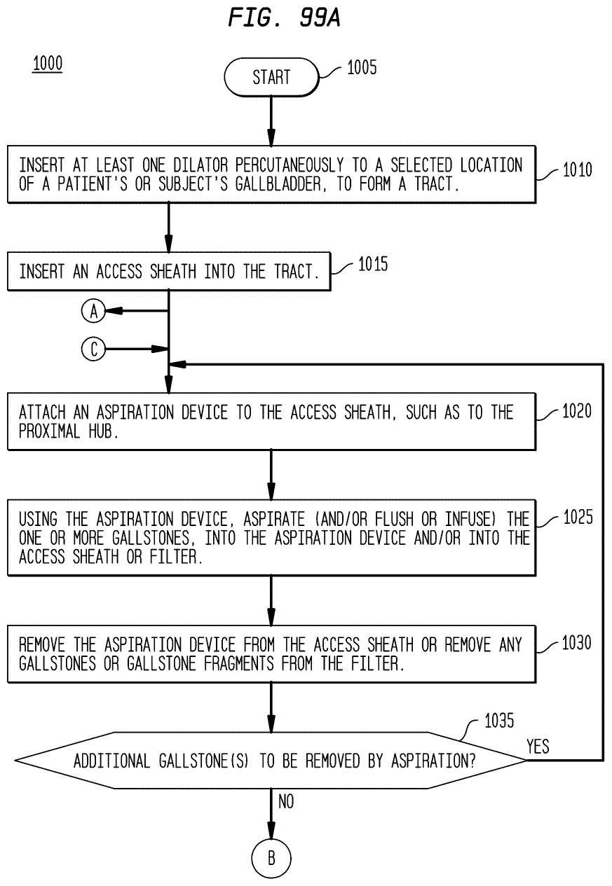

FIG. 93 is a first isometric view illustrating a representative second embodiment of a crushing or fracturing control apparatus 950 with a representative catheter 400F-400H embodiment and illustrated with a cage or basket 405H. FIG. 94 is a second isometric view illustrating the representative second embodiment of a crushing or fracturing control apparatus 950. FIG. 95 is a first partial cut-away view illustrating the representative second embodiment of a crushing or fracturing control apparatus 950 of FIG. 93 with a proximal portion of a representative catheter 400F-400H embodiment. FIG. 96 is a second partial cut-away view illustrating the representative second embodiment of a crushing or fracturing control apparatus of FIG. 93. FIG. 97 is a third partial cut-away view illustrating the representative second embodiment of a crushing or fracturing control apparatus of FIG. 93.

The crushing or fracturing control apparatus 950 comprises a housing 975, a control apparatus shaft 935 having a control apparatus shaft lumen 937, a catheter control assembly 992, a first handle 960, a second handle 955, a trigger actuator 940, and a trigger control tab 994. The catheter control assembly 992 comprises a catheter control linkage 965 and a crushing or fracturing pin 990. The crushing or fracturing pin 990 is arranged coaxially within, and moveable (slidable) longitudinally within, a lumen (not separately illustrated) of the catheter control linkage 965. The first handle 960 is slidable within the slot 958, and is coupled to and controls the catheter control linkage 965, to move the catheter control linkage 965 longitudinally, both distally and proximally. The second handle 955 is coupled to and controls the crushing or fracturing pin 990. The crushing or fracturing control apparatus 950 may also comprise a third (trigger) handle 945, and a control tab 970. The control apparatus shaft 935 has a control apparatus shaft lumen 937 for insertion of a catheter 400F-400H and a crushing or fracturing instrument 700-700S arranged coaxially within the inner catheter lumen 435 of the inner catheter manipulation shaft 420, 420A of the catheter 400F-400H.

The control apparatus shaft 935 is also insertable into an access sheath 800, also for example and without limitation. The crushing or fracturing control apparatus 950 is typically utilized and operated by medical personnel, holding the housing 950 and using the first, second and third handles 960, 955, 945, the control tab 970, and the trigger actuator 940. The various components of the crushing or fracturing control apparatus 950 may be comprised of a wide variety of materials, such as stainless steel, titanium, a medical/surgical grade polymer, and so on, for example and without limitation.

In operation, a catheter 400F-400H, with a crushing or fracturing instrument 700-700S arranged coaxially in the inner catheter lumen 435 of the catheter 400F-400H, is inserted into the control apparatus shaft lumen 937 of the control apparatus shaft 935. The inner catheter manipulation shaft (or member) 420, 420A of the catheter 400F-400H is arranged to engage with the catheter control linkage 965, and the crushing or fracturing instrument 700-700S is arranged to engage with the crushing or fracturing pin 990. With the first handle 960 in a first position 962 (illustrated in FIG. 94), the cage or basket 405G, 405H, 405J, 405K of the catheter 400F-400H is held within the outer catheter tube or member 415B, 415C of the catheter 400F-400H. In this arrangement, the crushing or fracturing instrument 700-700S is arranged coaxially in the inner catheter lumen 435 of the inner catheter manipulation shaft 420B within the catheter 400F-400H, which in turn is arranged coaxially within the control apparatus shaft lumen 937 of the control apparatus shaft 935. The control apparatus shaft 935 (having the catheter 400F-400H and crushing or fracturing instrument 700-700S) is then inserted into an access sheath 800 arranged in a patient's or subject's gallbladder, for example and without limitation.

With the control tab 970 engaged, the first handle 960 is moved within the slot 958 of the housing 975 into a second position 964, the catheter control linkage 965 engages with and pushes the inner catheter manipulation shaft 420B distally and longitudinally, advancing the cage or basket 405G, 405H, 405J, 405K out of the control apparatus shaft 935, as illustrated in FIG. 93. The expanded cage or basket 405G, 405H, 405J, 405K may then be utilized to capture a gallstone, as previously described. When a gallstone is then within the interior of the cage or basket 405G, 405H, 405J, 405K, the trigger actuator 940 pressed, engaging the trigger control tab 994 and moving the first handle 960 back toward the first position 962 (resulting in the first handle 960 being in a third position intermediate the first and second positions 962, 964, not separately illustrated), which retracts or pulls the inner catheter manipulation shaft 420B back into the outer catheter tube or member 415B, 415C (and control apparatus shaft 935), thereby retracting the struts 410 and contracting the cage or basket 405G, 405H, 405J, 405K around the gallstone. Depending on the size of the captured gallstone, the captured gallstone may then be removed from the patient or subject by withdrawing the crushing or fracturing shaft 935 of the crushing or fracturing control apparatus 950 from the access sheath 800. For a gallstone larger than the inner diameter of the central lumen 555 of the access sheath 800, the captured gallstone also may be crushed or fractured using one of the crushing or fracturing instruments 700-700S within the inner catheter shaft lumen 435.

In the event the gallstone is to be crushed or fractured, while the cage or basket 405G, 405H, 405J, 405K is contracted around and securing the gallstone, a crushing or fracturing instrument 700-700S may be advanced through the inner catheter shaft lumen 435 (such as illustrated in FIG. 54) to impact the gallstone, once or repeatedly, as needed to crush or fracture the gallstone, using the second handle 955 and manual force supplied by the user. The second handle 955 is typically maintained in a first locked position 966, as illustrated in FIG. 96, using a bias spring 980, so that the crushing or fracturing instrument 700-700S is not exposed. The user unlocks the second handle 955 by rotating it 90°, to a second position 968, as illustrated in FIG. 97. With the user then pushing on the second handle 955, the crushing or fracturing pin 990 (arranged coaxially within the catheter linkage 965) is advanced (moved distally longitudinally), which engages with the proximal end of the crushing or fracturing instrument 700-700S, which in turn advances (moves distally longitudinally) the crushing or fracturing tip 710-710S into the gallstone, thereby crushing or fracturing the gallstone. This results in force being transmitted from the user, through the crushing or fracturing pin 990 to the crushing or fracturing tip 710-710S and into the gallstone. When the second handle 955 is fully advanced in the second position 968, it may be locked into place with locking pins 995 seating into corresponding recesses 985 in the housing 975, as illustrated in FIG. 97. Concurrently or sequentially, the trigger actuator 940 is pressed one or more times by the user, resulting in further pulling of the inner catheter manipulation shaft 420B back into the outer catheter tube or member 415B (and control apparatus shaft 935), thereby further retracting the struts 410, resulting in a stepwise pulling of the gallstone toward the crushing or fracturing tip 710-710S of the crushing or fracturing instrument 700-700S, with the crushing or fracturing tip 710-710S chiseling into the gallstone and further helping to crush or fracture the gallstone. These operations using the second handle 955 and trigger actuator 940 may be repeated as may be necessary or advisable.

The fractured or crushed gallstone pieces also may be small enough to exit from the cage or basket 405G, 405H, 405J, 405K and be aspirated (or removed using the cage or basket 405G, 405H, 405J, 405K), as described above. Typically, the second crushing or fracturing control apparatus 950 is removed from the access sheath 800, and the gallstone fragments are aspirated, using the aspiration channel 574 having the filter assembly 515, as described above.

In addition to the various sizes described above, a catheter 400F-400H may be sized to fit within the first or second crushing or fracturing control apparatus 850, 950. In representative embodiments, both the control apparatus shafts 835, 935 may have an outer diameter of 0.310 inches (7.878 mm), an inner diameter of 0.272 inches (6.91 mm), and a length of 11.6 inches (29.46 cm), for example and without limitation. For the first crushing or fracturing control apparatus 850, the inner catheter manipulation shaft 420B may have an outer diameter of 0.197 inches (5.00 mm), an inner lumen (435) diameter of 0.177 inches (4.50 mm), and a length of 12.9 inches (29.46 cm), also for example and without limitation. For the second crushing or fracturing control apparatus 850, 950, the inner catheter manipulation shaft 420B may have an outer diameter of 0.22 inches (5.59 mm), an inner lumen (435) diameter of 0.177 inches (4.50 mm), and a length of 15.25 inches (29.46 cm), also for example and without limitation. The crushing or fracturing instrument 700-700S may be sized as described above to fit within the inner diameter of the lumen 435 of the inner catheter manipulation shaft 420B.

FIG. 98 is a front, elevational view illustrating a representative peristaltic pump 980 for use as part of any of the various systems 300-300J. In any of these embodiments, the access sheath 100, 100A, 200, 200A, 500, 500A, 600, 600A, 800, 900, without valves or with valves that only restrict inward flow, can be connected to a length of tubing 985 that can then be connected to peristaltic pump 980 to generate a source of vacuum. For example, the tubing may be coupled to or inserted within any of the one or more access ports 130, 130A, 135 or central, in-line port 152. Separately, this long tubing 985 can be connected to a filter and collection container. Further proximal to the filter and collection chamber, can be the vacuum source. With the filter and collection container in place, the vacuum source can look like a syringe, or a peristaltic pump, or a vacuum pump. The aspirated contents can then be dispensed into a dump bag or into a waste container or sorts.

In other embodiments of the access sheath 100, 100A, 200, 200A, 500, 500A, 600, 600A, 800, 900, the infusion or injection ports of the access sheath 100, 100A, 200, 200A, 500, 500A, 600, 600A, 800, 900, such as the one or more access ports 130, 130A, 135 or central, in-line port 152, can be connected to a pressurized saline bag or an infusion/injection pump to keep the gallbladder open and pressurized. In current procedures, this fluid is then allowed to exit the gallbladder around the access sheath 100, 100A, 200, 200A, 500, 500A, 600, 600A, 800, 900 through the access site, or through the central lumen of the tubing used for access. This creates a very messy procedure with the fluid being dumped onto the table and floor. Alternatively, our designs of the access sheath 100, 100A, 200, 200A, 500, 500A, 600, 600A, 800, 900 can have a dedicated “fluid exit” lumen (e.g., 165A or 165B) that may or may not be separate from the aspiration lumen. This “fluid exit” lumen can then be connected to a waste bin or dump bag.

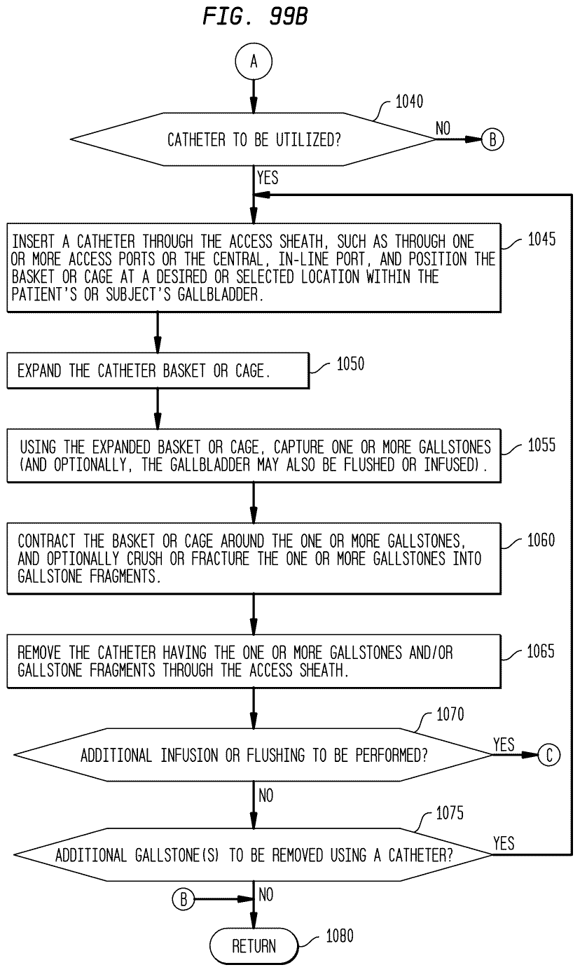

FIG. 99 is a flow chart illustrating representative methods 1000 for percutaneous removal of gallstones for the treatment of cholelithiasis, and provides a useful summary, with a first method for aspiration of gallstones comprising steps 1010-1035, a second method for capturing and crushing or fracturing gallstones comprising steps 1010, 1015, and 1040-1075, and a third method for both aspirating, capturing and crushing or fracturing gallstones comprising steps 1010-1075. The methodology begins, start step 1005, with the inserting, by medical personnel, of at least one dilator percutaneously to a selected location of a patient's or subject's gallbladder, to form a tract, step 1010. For example and without limitation, this step 1005 may comprise inserting of a series or sequence of dilators 145 having increasing diameters, or inserting a balloon dilator or balloon-tipped catheter followed by inflating the balloon, forming the tract. Also for example, this step 1005 may comprise inserting one or more guidewires through an existing gallbladder drain in the patient and removing the drain. The tubular shaft (e.g., 105, 505) of the access sheath 100, 100A, 200, 200A, 500, 500A, 600, 600A, 800, 900 is preloaded over a portion of a balloon dilation catheter (in an uninflated state). The balloon dilation catheter is then inserted over one guidewire so that the distal tip of the balloon is inside the gallbladder and the proximal end is externalized, and the balloon dilation catheter is inflated to dilate the tissue tract.

A tubular shaft (e.g., 105, 505, 505A) of an access sheath 100, 100A, 200, 200A, 500, 500A, 600, 600A, 800, 900 is then inserted into the tract, step 1015, either directly into the tract or over a dilator. For example, the tubular shaft (e.g., 105, 505, 505A) may be advanced over the balloon dilation catheter, and positioned with the distal end 140, 140A inside the gallbladder, followed by deflating the balloon dilation catheter and removing the balloon dilation catheter from the access sheath 100, 100A, 200, 200A, 500, 500A, 600, 600A, 800, 900, and inflating the sealing and anchoring balloon 560. Following insertion of the access sheath 100, 100A, 200, 200A, 500, 500A, 600, 600A, 800, 900, the methodology may proceed separately, sequentially or concurrently, with one or both of two parallel paths, with steps 1020-1035 being the first path and steps 1040-1075 being the second path, or vice-versa. For example and without limitation, steps 1020-1035 involving aspiration may proceed initially, and if aspiration alone is insufficient, the method may further proceed with steps 1040-1075, to help remove the gallstones using a catheter 400-400H.

Following insertion of the access sheath 100, 100A, 200, 200A, 500, 500A, 600, 600A, 800, 900 in step 1015, medical personnel may attach an aspiration device 250, 250A, 250B, 250C, 250D or dual aspiration and infusion assembly 650, 675 to the access sheath 100, 100A, 200, 200A, 500, 500A, 600, 600A, 800, 900, such as to the proximal hub 110, 110A, 510, 610 step 1020. Using the aspiration device 250, 250A, 250B, 250C, 250D or dual aspiration and infusion assembly 650, 675, in step 1025, medical personnel then aspirate (and/or flush or infuse) the one or more gallstones or gallstone fragments, into the filter 520 and/or aspiration device 250, 250A, 250B, 250C, 250D and/or into the access sheath 100, 100A, 200, 200A, 500, 500A, 600, 600A, 800, 900. The filter 520 or aspiration device 250, 250A, 250B, 250C, 250D is then removed from the access sheath 100, 100A, 200, 200A, 500, 500A, 600, 600A, 800, 900, step 1030, such as to empty any aspirated gallstones or fragments from the filter 520 or aspiration device 250, 250A, 250B, 250C, 250D. When there are additional gallstone(s) to be removed by aspiration, step 1035, the methodology returns to step 1020 and iterates. It should also be noted that before, after, or during the performance of any of these steps, other instruments (such as endoscopes, cameras, lithotripters, may also be inserted into the access sheath 100, 100A, 200, 200A, 500, 500A, 600, 600A, 800, 900 and utilized as part of the treatment process.

Before, concurrently, or subsequently to performing steps 1020-1035, following insertion of the access sheath 100, 100A, 200, 200A, 500, 500A, 600, 600A, 800, 900 in step 1015, in step 1040, medical personnel determine whether a catheter 400-400H should be utilized, and if so, in step 1045, medical personnel may then insert a catheter 400 400H through the access sheath 100, 100A, 200, 200A, 500, 500A, 600, 600A, 800, 900, such as through one or more access ports 130, 130A, 135 or the central, in-line port 152, and position the basket or cage 405-405K or other capturing device at a desired or selected location within the patient's or subject's gallbladder, such as by using a crushing or fracturing control apparatus 850, 950. The basket or cage 405-405K or other capturing device is then expanded, step 1050, and using the expanded basket or cage 405-405K or other capturing device, one or more gallstones are captured (and optionally, the gallbladder may also be flushed or infused), step 1055. The basket or cage 405-405K or other capturing device is then contracted around the one or more gallstones, and the one or more gallstones are optionally crushed or fractured into gallstone fragments, step 1060. The catheter 400-400H having the one or more gallstones and/or gallstone fragments is then removed through the access sheath 100, 100A, 200, 200A, 500, 500A, 600, 600A, 800, 900, step 1065. Medical personnel then determine whether additional infusion or flushing should be performed, step 1070, and if so, the methodology also returns to step 1020. When there are additional gallstone(s) to be removed using a catheter 400-400E, step 1075, the methodology returns to step 1045 and iterates. When there are no additional gallstone(s) to be removed in both steps 1035 and 1075, the methods for percutaneous removal of gallstones for the treatment of cholelithiasis may end, return step 1080. As part of step 1080, another drainage catheter is typically inserted over one of the guidewires and the drainage catheter is secured within the subject gallbladder.

As mentioned above, the representative embodiments of the system 300 300J, and any of its various components such as an access sheath 100, 100A, 200, 200A, 500, 500A, 600, 600A, 800, 900, an aspiration device 250, 250A, 250B, 250C, 250D, and a catheter 400-400H etc., may have any size (height, width, depth), shape, or form factor suitable for use with percutaneous removal of gallstones for the treatment of cholelithiasis, in addition to those illustrated and described above, and all such variations are considered equivalent and within the scope of the disclosure. In addition, the representative embodiments of the access sheaths 100, 100A, 200, 200A, 500, 500A, 600, 600A, 800, 900, aspiration devices 250, 250A, 250B, 250C, 250D, and catheters 400-400E illustrate different combinations of features and elements, with any and all mixing and matching of any of the various features and elements and any and all combinations of any of the various features and elements are within the scope hereof.

The access sheaths 100, 100A, 200, 200A, 500, 500A, 600, 600A, 800, 900, aspiration devices 250, 250A, 250B, 250C, 250D, and catheters 400-400E, and other components may be fabricated in a wide variety of ways, including integrally formed (e.g., injection molded, 3D printed) or assembled from separate components (e.g., using any suitable fasteners or adhesives, not separately illustrated), and all such variations are considered equivalent and within the scope of the disclosure. The access sheaths 100, 100A, 200, 200A, 500, 500A, 600, 600A, 800, 900, aspiration devices 250, 250A, 250B, 250C, 250D, and catheters 400-400E, and other components, may be implemented using any suitable material, and may be opaque or transparent, with suitable materials including any rigid (or semi-flexible) polymer or plastic, such as polyvinylchloride (PVC), polystyrene, polyacrylate, polytetrafluoroethylene (PTFE or Teflon), nylon, polycarbonates, polyesters, carbon fiber, glass, silicone, silicone rubber, a metal, an alloy, etc., for example and without limitation, and all such variations are considered equivalent and within the scope of the disclosure. The access sheaths 100, 100A, 200, 200A, 500, 500A, 600, 600A, 800, 900, aspiration devices 250, 250A, 250B, 250C, 250D, and catheters 400-400E, and other components also may have one or more coatings (not separately illustrated), such as an antibiotic or antimicrobial coating, for example and without limitation.

Representative examples of suitable polymers include, but are not limited to, fluorinated polymers or copolymers such as poly(vinylidene fluoride), poly(vinylidene fluoride-co-hexafluoropropene), poly(tetrafluoroethylene), and expanded poly(tetrafluoroethylene); poly(sulfone); poly(N-vinyl pyrrolidone); poly(aminocarbonates); poly(iminocarbonates); poly(anhydride-co-imides), poly(hydroxyvalerate); poly(L-lactic acid); poly(L-lactide); poly(caprolactones); poly(lactide-co-glycolide); poly(hydroxybutyrates); poly(hydroxybutyrate-co-valerate); poly(dioxanones); poly(orthoesters); poly(anhydrides); poly(glycolic acid); poly(glycolide); poly(D, L-lactic acid); poly(D, L-lactide); poly(glycolic acid-cotrimethylene carbonate); poly(phosphoesters); poly(phosphoester urethane); poly(trimethylene carbonate); poly(iminocarbonate); poly(ethylene); and any derivatives, analogs, homologues, congeners, salts, copolymers and combinations thereof.

The polymers may also include, but are not limited to, poly(propylene) co-poly(ether-esters) such as, for example, poly(dioxanone) and poly(ethylene oxide)/poly(lactic acid); poly(anhydrides), poly(alkylene oxalates); poly(phosphazenes); poly(urethanes); silicones; silicone rubber; poly(esters); poly(olefins); copolymers of poly(isobutylene); copolymers of ethylene-alphaolefin; vinyl halide polymers and copolymers such as poly(vinyl chloride); poly(vinyl ethers) such as, for example, poly(vinyl methyl ether); poly(vinylidene halides) such as, for example, poly(vinylidene chloride); poly(acrylonitrile); poly(vinyl ketones); poly(vinyl aromatics) such as poly(styrene); poly(vinyl esters) such as poly(vinyl acetate); copolymers of vinyl monomers and olefins such as poly(ethylene-co-vinyl alcohol) (EVAL), copolymers of acrylonitrile-styrene, ABS resins, and copolymers of ethylene-vinyl acetate; and any derivatives, analogs, homologues, congeners, salts, copolymers and combinations thereof.

The polymers may further include, but are not limited to, poly(amides) such as Nylon 66 and poly(caprolactam); alkyd resins; poly(carbonates); poly(oxymethylenes); poly(imides); poly(ester amides); poly(ethers) including poly(alkylene glycols) such as, for example, poly(ethylene glycol) and poly(propylene glycol); epoxy resins; polyurethanes;

rayon; rayon-triacetate; biomolecules such as, for example, fibrin, fibrinogen, starch, poly(amino acids); peptides, proteins, gelatin, chondroitin sulfate, dermatan sulfate (a copolymer of D-glucuronic acid or L-iduronic acid and N-acetyl-D-galactosamine), collagen, hyaluronic acid, and glycosaminoglycans; other polysaccharides such as, for example, poly(N-acetylglucosamine), chitin, chitosan, cellulose, cellulose acetate, cellulose butyrate, cellulose acetate butyrate, cellophane, cellulose nitrate, cellulose propionate, cellulose ethers, and carboxymethylcellulose; and any derivatives, analogs, homologues, congeners, salts, copolymers and combinations thereof.

The present disclosure is to be considered as an exemplification of the principles of the invention and is not intended to limit the invention to the specific embodiments illustrated. In this respect, it is to be understood that the invention is not limited in its application to the details of construction and to the arrangements of components set forth above and below, illustrated in the drawings, or as described in the examples. Systems, methods and apparatuses consistent with the present invention are capable of other embodiments and of being practiced and carried out in various ways.

Although the invention has been described with respect to specific embodiments thereof, these embodiments are merely illustrative and not restrictive of the invention. In the description herein, numerous specific details are provided, such as examples of electronic components, electronic and structural connections, materials, and structural variations, to provide a thorough understanding of embodiments of the present invention.

One skilled in the relevant art will recognize, however, that an embodiment of the invention can be practiced without one or more of the specific details, or with other apparatus, systems, assemblies, components, materials, parts, etc. In other instances, well-known structures, materials, or operations are not specifically shown or described in detail to avoid obscuring aspects of embodiments of the present invention. In addition, the various Figures are not drawn to scale and should not be regarded as limiting.

Reference throughout this specification to “one embodiment”, “an embodiment”, or a specific “embodiment” means that a particular feature, structure, or characteristic described in connection with the embodiment is included in at least one embodiment of the present invention and not necessarily in all embodiments, and further, are not necessarily referring to the same embodiment. Furthermore, the particular features, structures, or characteristics of any specific embodiment of the present invention may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

It will also be appreciated that one or more of the elements depicted in the Figures can also be implemented in a more separate or integrated manner, or even removed or rendered inoperable in certain cases, as may be useful in accordance with a particular application. Integrally formed combinations of components are also within the scope of the invention, particularly for embodiments in which a separation or combination of discrete components is unclear or indiscernible. In addition, use of the term “coupled” herein, including in its various forms such as “coupling” or “couplable”, means and includes any direct or indirect electrical or structural coupling, connection or attachment, or adaptation or capability for such a direct or indirect electrical or structural coupling, connection or attachment, including integrally formed components and components which are coupled via or through another component.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. In addition, every intervening sub-range within range is contemplated, in any combination, and is within the scope of the disclosure. For example, for the range of 5-10, the sub-ranges 5-6, 5-7, 5-8, 5-9, 6-7, 6-8, 6-9, 6-10, 7-8, 7-9, 7-10, 8-9, 8-10, and 9-10 are contemplated and within the scope of the disclosed range.

Furthermore, any signal arrows in the drawings/Figures should be considered only exemplary, and not limiting, unless otherwise specifically noted. Combinations of components of steps will also be considered within the scope of the present invention, particularly where the ability to separate or combine is unclear or foreseeable. The disjunctive term “or”, as used herein and throughout the claims that follow, is generally intended to mean “and/or”, having both conjunctive and disjunctive meanings (and is not confined to an “exclusive or” meaning), unless otherwise indicated. As used in the description herein and throughout the claims that follow, “a”, “an”, and “the” include plural references unless the context clearly dictates otherwise. Also as used in the description herein and throughout the claims that follow, the meaning of “in” includes “in” and “on” unless the context clearly dictates otherwise.

The foregoing description of illustrated embodiments of the present invention, including what is described in the summary or in the abstract, is not intended to be exhaustive or to limit the invention to the precise forms disclosed herein. From the foregoing, it will be observed that numerous variations, modifications and substitutions are intended and may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

1-381. (canceled)

382. A system for percutaneous removal of gallstones from a human or veterinary subject for the treatment of cholelithiasis, the system comprising:
an access sheath; and
a catheter removably insertable into the access sheath;
wherein the access sheath comprises:
a proximal hub;
a tubular shaft having a first, proximal end and a second, distal end, the first, proximal end of the tubular shaft coupled to the proximal hub, the tubular shaft having a longitudinal axis and a central lumen arranged longitudinally, the tubular shaft comprising:
an inner shaft wall forming the central lumen;
an outer shaft wall arranged coaxially with and spaced apart radially from the inner shaft wall to form, in between the inner and outer shaft walls, one or more peripheral lumens; and
an inflation lumen arranged between the inner and outer shaft walls and spaced apart from the second, distal end; and
an access sheath tip coupled to the second, distal end of the tubular shaft, the access sheath tip having a beveled tip and a plurality of infusion ports, the plurality of infusion ports arranged radially and spaced apart from the beveled tip and in fluid communication with the one or more peripheral lumens, the access sheath tip further comprising a plurality of tabs insertable between the inner shaft wall and the outer shaft wall.

383. The system of claims 382, wherein the access sheath further comprises:
an inflatable sealing and anchoring balloon arranged spaced apart from the second, distal end and proximally to the plurality of infusion ports, the inflatable sealing and anchoring balloon coupled radially around the outer wall, the inflatable sealing and anchoring balloon having a balloon lumen arranged in fluid communication with the inflation lumen.

384. The system of claims 382, wherein the proximal hub comprises:
a hub housing having a hub lumen, the hub lumen arranged in fluid communication with the central lumen;
a filter assembly removably coupleable to the hub housing, the filter assembly arranged in fluid communication with the central lumen;
a first port integrally formed with or coupled to the hub housing, the first port arranged in fluid communication with the one or more peripheral lumens; and
a second port integrally formed with or coupled to the hub housing, the second port arranged in fluid communication with the inflation lumen; and
a third port integrally formed with or coupled to the hub housing, the third port arranged in fluid communication with the central lumen for insertion of the catheter into the central lumen.

385. The system of claim 384, wherein the filter assembly comprises:
a filter canister removably coupleable to the hub housing, the filter canister having a filter canister lumen; and
a filter arranged within the filter canister and in fluid communication with the hub lumen, the filter forming a filter chamber within the filter canister.

386. The system of claim 385, wherein the proximal hub further comprises:
an infusion path tubing integrally formed with or coupled to the first port, the infusion path tubing arranged in fluid communication with the one or more peripheral lumens;
a first valve integrally formed with or coupled to the infusion path tubing;
a second valve integrally formed with or coupled to the hub housing and in fluid communication with the filter canister lumen;
a connector coupled to the first valve and to the second valve, the connector removably coupleable to an aspiration device.

387. The system of claim 385, wherein the proximal hub further comprises:
an infusion path tubing integrally formed with or coupled to the first port, the infusion path tubing arranged in fluid communication with the one or more peripheral lumens;
one or more first valves integrally formed with or coupled to the infusion path tubing;
a second valve integrally formed with or coupled to the filter assembly or to the hub housing and in fluid communication with the filter canister lumen;
a return path tubing integrally formed with or coupled to the second valve;
a connecting tubing having a third valve coupled between and in fluid communication with the infusion path tubing and the return path tubing;
a first connector coupled to the infusion path tubing; and
a second connector coupled to the return path tubing.

388. The system of claim 387, further comprising:
a first aspiration device removably coupleable to the first connector, the first aspiration device comprising a first syringe tube body and a first plunger moveable within the first syringe tube body; and
a second aspiration device removably coupleable to the second connector, the second aspiration device comprising a second syringe tube body and a second plunger moveable within the second syringe tube body.

389. The system of claim 388, further comprising:
a first rack gear coupled to the first plunger;
a second rack gear coupled to the second plunger;
a pinion gear abutting and moveably engaging the first and second rack gears, the pinion gear arranged to advance the first plunger within the first syringe tube body while concurrently retracting the second plunger from the second syringe tube body and to advance the second plunger within the second syringe tube body while concurrently retracting the first plunger from the first syringe tube body; and
a clamping assembly coupled to the pinion gear and to the first and second syringe tube bodies.

390. The system of claim 388, further comprising:
a pump coupled to the first and second plungers, the pump arranged to advance the first plunger within the first syringe tube body while concurrently retracting the second plunger from the second syringe tube body and to advance the second plunger within the second syringe tube body while concurrently retracting the first plunger from the first syringe tube body.

391. The system of claim 382, wherein the catheter has a longitudinal dimension and a transverse dimension, wherein the catheter comprises:
an outer catheter tube having an outer catheter lumen and an outer catheter tube distal end;
an inner catheter manipulation shaft moveable longitudinally and rotatably within the outer catheter lumen, the inner catheter manipulation shaft having an inner catheter lumen and an inner catheter shaft distal end; and
a basket or cage coupled to the inner catheter manipulation shaft, the basket or cage comprising a plurality of struts.

392. The system of claim 391, wherein the plurality of struts are arranged longitudinally and coupled to a strut ring at a first end of the basket or cage and the plurality of struts are coupled to the distal end of the inner catheter manipulation at a second end of the basket or cage.

393. The system of claim 392, wherein the catheter further comprises:
an atraumatic, molded end cap coupled to the strut ring.

394. The system of claim 391, further comprising:
a crushing or fracturing instrument removably insertable into the inner catheter lumen and moveable longitudinally within the inner catheter lumen and to extend out of the inner catheter lumen and the outer catheter tube.

395. The system of claim 394, wherein the crushing or fracturing instrument comprises:
a crushing or fracturing shaft; and
a crushing or fracturing tip integrally formed with or couped at a first end to the crushing or fracturing shaft, the crushing or fracturing tip having one or more crushing or fracturing spears at a second, distal end, each crushing or fracturing spear, of the one or more crushing or fracturing spears, comprises at least two beveled and chamfered inner edges terminating in a sharp point.

396. The system of claim 394, wherein the crushing or fracturing instrument comprises:
a crushing or fracturing shaft; and
a crushing or fracturing tip integrally formed with or couped at a first end to the crushing or fracturing shaft, the crushing or fracturing tip having at least one configuration selected from the group consisting of: an angled configuration, a pointed configuration, a sharp-edged or beveled configuration, a bullet configuration, a spherical or “button” configuration, a concave tri-tip configuration, a spear configuration, a concave spear configuration, a core drilling configuration, a fluted configuration, a hollow configuration, and combinations thereof.

397. The system of claim 394, further comprising:
a crushing or fracturing control apparatus, the crushing or fracturing control apparatus comprises:
a housing;
a control apparatus shaft coupled to the housing, the control apparatus shaft having a control apparatus shaft lumen configured to hold the catheter and the crushing or fracturing instrument arranged within the inner catheter lumen;
a catheter control assembly arranged within the housing, the catheter control assembly comprising:
a catheter control actuator: and
a crushing or fracturing pin;
a first handle pivotably coupled to the housing;
a control linkage lever pivotably coupled to the first handle and pivotably coupled to the catheter control actuator;
a crushing or fracturing spring coupled around the crushing or fracturing pin; and
a second handle coupled to the crushing or fracturing pin.

398. The system of claim 398, further comprising:
a crushing or fracturing control apparatus, the crushing or fracturing control apparatus comprising:
a housing having a housing slot;
a control apparatus shaft coupled to the housing, the control apparatus shaft having a control apparatus shaft lumen configured to hold the catheter and the crushing or fracturing instrument arranged within the catheter;
a catheter control assembly arranged within the housing, the catheter control assembly comprising:
a catheter control linkage; and
a crushing or fracturing pin;
a first handle coupled to the catheter control linkage and slidable within the housing slot;
a second handle coupled to the crushing or fracturing pin; and
a trigger actuator pivotably coupled to the housing and further coupled to engage the catheter control linkage.

399. The system of claim 382, further comprising:
an aspiration device, comprising:
a syringe tube body having an interior lumen;
an extended distal tip coupled to or integrally formed with the syringe tube body;
a plunger moveable within the interior lumen of the syringe tube body; and
a syringe valve coupled to the extended distal tip or coupled to the syringe tube body, the syringe valve comprising at least one valve or adapter selected from the group consisting of: a duckbill valve; a cross-slit valve; a stopcock valve, a gate valve, a rotary valve; a ball valve; a luer valve; a Tuohy-Borst adapter; and combinations thereof.

400. A system for percutaneous removal of gallstones from a human or veterinary subject for the treatment of cholelithiasis, the system comprising:
an access sheath; and
a catheter removably insertable into the access sheath, the catheter having a longitudinal dimension and a transverse dimension, the catheter comprising:
an outer catheter tube having an outer catheter lumen and an outer catheter tube distal end;
an inner catheter manipulation shaft moveable longitudinally and rotatably within the outer catheter lumen, the inner catheter manipulation shaft having an inner catheter lumen and an inner catheter shaft distal end; and
a basket or cage coupled to the inner catheter manipulation shaft, the basket or cage comprising a plurality of struts arranged longitudinally, wherein the plurality of struts are coupled to a strut ring at a first end of the basket or cage and the plurality of struts are coupled to the distal end of the inner catheter manipulation at a second end of the basket or cage.

401. The system of claim 400, wherein the catheter further comprises:
an atraumatic, molded end cap coupled to the strut ring.

402. The system of claim 400, wherein the inner catheter manipulation shaft is further moveable longitudinally to extend out of the outer catheter tube; wherein the basket or cage is moveable, in a contracted or compressed state, with the inner catheter manipulation shaft, both longitudinally and rotatably within the outer catheter lumen; and wherein the basket or cage is further moveable longitudinally to extend out of the distal end of the outer catheter tube, and when the basket or cage is fully extended out of the distal end of the outer catheter tube, the basket or cage has an expanded state.

403. The system of claim 400, wherein the access sheath comprises:
a tubular shaft having a first, proximal end and a second, distal end, the tubular shaft having a longitudinal axis and a central lumen arranged longitudinally; and
a proximal hub coupled to the first end of the tubular shaft.

404. The system of claim 403, wherein the tubular shaft comprises:
an inner shaft wall forming the central lumen;
an outer shaft wall arranged coaxially with and spaced apart radially from the inner shaft wall to form, in between the inner and outer shaft walls, one or more peripheral lumens; and
an inflation lumen arranged between the inner and outer shaft walls.

405. The system of claim 404, wherein the outer shaft wall further comprises:
a plurality of infusion ports and at least one inflation port, the plurality of infusion ports arranged radially around a periphery of the outer shaft wall and spaced apart from the second, distal end, the inflation lumen arranged spaced apart from the second, distal end, and the plurality of infusion ports in fluid communication with the one or more peripheral lumens.

406. The system of claim 404, wherein the access sheath further comprises:
an access sheath tip coupled to the second, distal end of the tubular shaft, the access sheath tip having a beveled tip and a plurality of infusion ports, the plurality of infusion ports arranged radially and spaced apart from the beveled tip and in fluid communication with the one or more peripheral lumens, the access sheath tip further comprising a plurality of tabs insertable between the inner shaft wall and the outer shaft wall.

407. The system of claims 406, wherein the access sheath further comprises:
an inflatable sealing and anchoring balloon arranged spaced apart from the second, distal end and proximally to the plurality of infusion ports, the inflatable sealing and anchoring balloon coupled radially around the outer wall, the inflatable sealing and anchoring balloon having a balloon lumen arranged in fluid communication with the inflation lumen.

408. The system of claims 403, wherein the proximal hub comprises:
a hub housing having a hub lumen, the hub lumen arranged in fluid communication with the central lumen;
a filter assembly removably coupleable to the hub housing, the filter assembly arranged in fluid communication with the central lumen;
a first port integrally formed with or coupled to the hub housing, the first port arranged in fluid communication with the one or more peripheral lumens; and
a second port integrally formed with or coupled to the hub housing, the second port arranged in fluid communication with the inflation lumen; and
a third port integrally formed with or coupled to the hub housing, the third port arranged in fluid communication with the central lumen for insertion of the catheter into the central lumen.

409. The system of claim 408, wherein the filter assembly comprises:
a filter canister removably coupleable to the hub housing, the filter canister having a filter canister lumen; and
a filter arranged within the filter canister and in fluid communication with the hub lumen, the filter forming a filter chamber within the filter canister.

410. The system of claim 400, further comprising:
a crushing or fracturing instrument removably insertable into the inner catheter lumen and moveable longitudinally within the inner catheter lumen and to extend out of the inner catheter lumen and the outer catheter tube.

411. The system of claim 410, wherein the crushing or fracturing instrument comprises:
a crushing or fracturing shaft; and
a crushing or fracturing tip integrally formed with or couped at a first end to the crushing or fracturing shaft, the crushing or fracturing tip having one or more crushing or fracturing spears at a second, distal end, each crushing or fracturing spear, of the one or more crushing or fracturing spears, comprising at least two beveled and chamfered inner edges terminating in a sharp point.

412. The system of claim 410, wherein the crushing or fracturing instrument comprises:
a crushing or fracturing shaft; and
a crushing or fracturing tip integrally formed with or couped at a first end to the crushing or fracturing shaft, the crushing or fracturing tip having at least one configuration selected from the group consisting of: an angled configuration, a pointed configuration, a sharp-edged or beveled configuration, a bullet configuration, a spherical or “button” configuration, a concave tri-tip configuration, a spear configuration, a concave spear configuration, a core drilling configuration, a fluted configuration, a hollow configuration, and combinations thereof.

413. The system of claim 410, further comprising:
a crushing or fracturing control apparatus, the crushing or fracturing control apparatus configured to move the inner catheter manipulation shaft longitudinally and to move the crushing or fracturing instrument longitudinally within the inner catheter lumen and to extend the crushing or fracturing instrument out of the inner catheter lumen and the outer catheter tube.

414. The system of claim 406, further comprising:
an aspiration device, comprising:
a syringe tube body having an interior lumen;
an extended distal tip coupled to or integrally formed with the syringe tube body;
a plunger moveable within the interior lumen of the syringe tube body; and
a syringe valve coupled to the extended distal tip or coupled to the syringe tube body, the syringe valve comprising at least one valve or adapter selected from the group consisting of: a duckbill valve; a cross-slit valve; a stopcock valve, a gate valve, a rotary valve; a ball valve; a luer valve; a Tuohy-Borst adapter; and combinations thereof.

415. A system for percutaneous removal of gallstones from a human or veterinary subject for the treatment of cholelithiasis, the system comprising:
an access sheath, the access sheath comprising a proximal hub and a tubular shaft, the proximal hub comprising a hub housing having a hub lumen and a filter assembly removably coupleable to the hub housing; the tubular shaft having a first, proximal end and a second, distal end, the first, proximal end of the tubular shaft coupled to the proximal hub, the tubular shaft having a longitudinal axis and a central lumen arranged longitudinally and in fluid communication with the filter assembly, the tubular shaft comprising: an inner shaft wall forming the central lumen, an outer shaft wall arranged coaxially with and spaced apart radially from the inner shaft wall to form one or more peripheral lumens in between the inner and outer shaft walls; and an inflation lumen arranged between the inner and outer shaft walls and spaced apart from the second, distal end; the access sheath further comprising a beveled tip and a plurality of infusion ports, the plurality of infusion ports arranged radially and spaced apart from the beveled tip and in fluid communication with the one or more peripheral lumens;
a catheter removably insertable into the central lumen of the access sheath, the catheter having a longitudinal dimension and a transverse dimension, the catheter comprising: an outer catheter tube having an outer catheter lumen and an outer catheter tube distal end, an inner catheter manipulation shaft moveable longitudinally and rotatably within the outer catheter lumen, the inner catheter manipulation shaft having an inner catheter lumen and an inner catheter shaft distal end; and a basket or cage coupled to the inner catheter manipulation shaft, the basket or cage comprising a plurality of struts arranged longitudinally, wherein the plurality of struts are coupled to a strut ring at a first end of the basket or cage and the plurality of struts are coupled to the distal end of the inner catheter manipulation at a second end of the basket or cage;
an aspiration device removably coupleable to the access sheath; and
a crushing or fracturing instrument removably insertable into the inner catheter lumen and moveable longitudinally within the inner catheter lumen and to extend out of the inner catheter lumen and the outer catheter tube.
